(19) United States
(12) Patent Application Publication
(10) Pub. No.: US 2026/0159812 A1
(43) Pub. Date: Jun. 11, 2026

(54) REPROGRAMMING OF ADULT CARDIAC FIBROBLASTS INTO CARDIOMYOCYTES USING PHF7

(71) Applicant: The Board of Regents of The University of Texas System, Austin, TX (US)

(72) Inventors: Glynnis GARRY, Dallas, TX (US); Huanyu ZHOU, Dallas, TX (US); Eric OLSON, Dallas, TX (US)

(73) Assignee: The Board of Regents of The University of Texas System, Austin, TX (US)

(21) Appl. No.: 18/706,525

(22) PCT Filed: Oct. 28, 2022
(86) PCT No.: PCT/US2022/078854

§ 371 Date: May 1, 2024

Related U.S. Application Data
(60) Provisional application No. 63/274,229, filed on Nov. 1, 2021.

(51) Int. Cl. C12N 5/077; A61K 31/21; A61K 31/616; A61K 31/7105; A61K 38/17; A61K 38/45; A61K 45/06; A61K 48/00; A61P 9/10; C12N 15/86

(52) U.S. Cl. CPC C12N 5/0657; A61K 31/21; A61K 31/616; A61K 31/7105; A61K 38/1709; A61K 38/45; A61K 45/06; A61K 48/005; A61P 9/10; C12N 15/86; C12Y 207/11001; C12N 2501/60; C12N 2501/727; C12N 2506/1307; C12N 2510/00; C12N 2740/10043

(57) ABSTRACT

The present disclosure involves the use of the reprogramming factor PHF7, and optionally further using TBX5, MEF2C, MGATA4, HAND2, MYCD, and/or phosphorylated AKT1 to reprogram adult non-cardiomyocytes, such as cardiac fibroblasts into cardiomyocytes, both in vitro and in vivo in adult human and murine models of myocardial infarction and/or heart failure. Such methods find particular use in the treatment of patients' post-myocardial infarction and/or heart failure to prevent, limit, or reverse scarring and to promote myocardial repair.

### PRIORITY CLAIM

This application claims benefit of priority to U.S. Provisional Application Ser. No. 63/274,229, filed Nov. 1, 2021, the entire contents of which are hereby incorporated by reference.

### BACKGROUND

The invention was made with government support under grant nos. HL-130253, HL-138426, HD-087351 and 5T32HL125247-04 awarded by the National Institutes of Health. The government owns certain rights in the invention.

### INCORPORATION OF SEQUENCE LISTING

The sequence listing that is contained in the file named “UTFD-P3978WO-SequenceListing.xml”, which is 47 KB (as measured in Microsoft Windows®) and was created on Oct. 27, 2022, is filed herewith by electronic submission and is incorporated by reference herein.

### 1. FIELD

The present disclosure relates generally to the fields of cardiology, developmental biology and molecular biology. More particularly, it concerns cell fate conversion, epigenetic regulation, and cellular physiology in cardiomyocytes. Specifically, the invention relates to the use of PHF7 to reprogram cardiac fibroblasts into cardiomyocytes and use of the same in the prevention of scar formation and induction of repair in the heart following myocardial infarction.

### 2. DESCRIPTION OF RELATED ART

In recent years, direct reprogramming of resident cardiac fibroblasts (CFs) to induced cardiac-like myocytes (iCLMs) has emerged as a promising therapeutic strategy to induce remuscularization of the injured heart. However, epigenetic barriers severely limit conversion efficiency of adult fibroblasts, thus constraining the utility of this approach. In development, chromatin remodeling complexes function in a highly cell-specific manner to dictate cellular fate by either restructuring the nucleosome or directly modifying histones (Wamstad et al., 2012). In cardiovascular tissues, the SWI/SNF complex, in particular, interacts synergistically with cardiac TFs to dictate chromatin accessibility and cardiac gene expression (Lickert et al., 2004; Sun et al., 2018; Hota et al., 2019; Hang et al., 2010; Takeuchi & Bruneau 2009). Cardiac chromatin remodeling complexes conceivably hold tremendous potential to redefine the epigenetic landscape of somatic cells, yet overexpression of select remodelers has shown little or no benefit (Ieda et al., 2010; Christoforou et al., 2013). Despite the central influence of epigenomic structure on reprogramming, the enhancer landscape of iCLMs has only recently been defined and limited efforts have been dedicated to an enhanced understanding of the role of chromatin regulatory factors and complexes in mediating this cellular process (Hashimoto et al., 2019; Liu et al., 2016; Stone et al., 2019; Zhou et al., 2018; Zhou et al., 2016).

### SUMMARY

Thus, in accordance with the present disclosure, there is provided a method of reprogramming a cardiac fibroblast comprising contacting said cardiac fibroblast with PHF7. The method may further comprise contacting said fibroblast with both PHF7 and TBX5, and/or may further comprise contacting said cardiac fibroblast with PHF7, MEF2C, and TBX5, and/or may further comprise delivering PHF7 with GATA4, MEF2C, and TBX5 proteins to said cardiac fibroblast; and/or may further comprise contacting said cardiac fibroblast with HAND2, and/or may further comprise contacting said cardiac fibroblast with MYCD, and/or may further comprise contacting said cardiac fibroblast with phosphorylated AKT1. The method may further comprise contacting said cardiac fibroblast with an anti-inflammatory agent, small molecule, or micro-RNA.

Contacting may comprise delivering PHF7 proteins to said cardiac fibroblast, and/or may further comprise delivering PHF7 and TBX5 proteins to said cardiac fibroblasts; and/or PHF7, MEF2C, and TBX5 proteins to said cardiac fibroblast, and/or may further comprise delivering PHF7 with GATA4, MEF2C, and TBX5 proteins to said cardiac fibroblast, and/or may further comprise delivering HAND2 proteins to said cardiac fibroblast, and/or may further comprise delivering said MYCD proteins to said cardiac fibroblast, and/or may further comprise delivering phosphorylated AKT1 proteins to said cardiac fibroblast. Any or all of PHF7, MYCD, GATA4, HAND2, MEF2C, TBX5, and/or phosphorylated AKT1 proteins may comprise a heterologous cell permeability peptide (CPP).

Contacting may comprise delivering PHF7 expression cassettes to said cardiac fibroblast, and/or may further comprise delivering one or more expression cassette encoding PHF7 and TBX5, and/or may further comprise PHF7, MEF2C, and TBX5 to said cardiac fibroblast, and/or may further comprise delivering an expression cassette encoding PHF7, MYCD, GATA4, HAND2, MEF2C, TBX5, and/or phosphorylated AKT1 to said cardiac fibroblast. The TBX5 encoding nucleic acid segment may be in the same expression cassette as PHF7. The MEF2C encoding nucleic acid segment may be in the same expression cassette as either or both of PHF7 and TBX5, such as wherein PHF7, MYCD, GATA4, HAND2, MEF2C, TBX5, and/or phosphorylated AKT1 encoding nucleic acid segments are in the same expression cassette. The MEF2C encoding nucleic acid segment may be in a distinct expression cassette from either or both of PHF7 and TBX5, such as wherein PHF7, MYCD, GATA4, HAND2, MEF2C, TBX5, and/or phosphorylated AKT1 encoding nucleic acid segments are in distinct expression cassettes. The expression cassette or cassettes may be comprised in one or more replicable vectors, such as one or more viral vectors or one or more non-viral vectors. The one or more viral vectors may be one or more adeno-associated virus (AAV), non-integrated lentivirus, adenoviral vectors or retroviral vectors, and the one or more replicable vectors may be modified RNA delivery vectors, including one or more non-viral vectors disposed in a lipid delivery vehicle.

Also provided is a method of treating a subject having suffered a myocardial infarct (MI) and heart failure comprising delivering to said subject PHF7. The method may further comprise delivering to said subject PHF7 and TBX5, and/or may further comprise delivering to said subject PHF7, MEF2C, and TBX5, and/or may further comprise delivering to said subject PHF7, GATA4, MEF2C and TBX5, and/or may further comprise delivering to said subject HAND2, and/or may further comprise delivering to said subject MYCD, and/or may further comprise delivering to said subject phosphorylated AKT1. Delivering may comprise administration of PHF7 proteins and/or PHF7 and TBX5 proteins, such as wherein one or both of PHF7 and TBX5 proteins comprise a heterologous cell permeability peptide (CPP), including wherein one, two, three, four, five or all six of MCYD, phosphorylated AKT1, GATA4, HAND2, MEF2C, and/or TBX5 proteins comprise a heterologous cell permeability peptide (CPP). The method may further comprise administering to said subject an anti-inflammatory agent, small molecule, micro-RNA, oxygen, aspirin, nitroglycerin, a fibrinolytic, percutaneous coronary intervention, and/or surgical correction through coronary bypass. The MI may be non-ST-elevated MI or ST-elevated MI.

Delivering may comprise administering PHF7 as an expression cassette to injured myocardium or scar, such as by systemic, intracardiac, or intracoronary injection. The method may further comprise administering one or more expression cassettes encoding PHF7 and TBX5 to said myocardium; and/or PHF7, MEF2C, and TBX5 to said myocardium, such as by intracardiac or intracoronary injection, and/or may further comprising administering an expression cassette encoding PHF7 with a combination of expression cassettes encoding GATA4, HAND2, MEF2C, TBX5, MYCD, and/or phosphorylated AKT1, such as by intracardiac or intracoronary injection. The PHF7 encoding nucleic acid segment may be in the same expression cassette as either or both of MEF2C and TBX5, such as wherein a PHF7 encoding nucleic acid segment is in the same expression cassette as either or both of GATA4, HAND2, MEF2C, TBX5, MYCD, and/or phosphorylated AKT1. The MEF2C encoding nucleic acid segment may be in a distinct expression cassette from either or both of PHF7 and TBX5, such as wherein the PHF7 encoding nucleic acid segment is in a distinct expression cassette from either or all of GATA4, HAND2, MEF2C, TBX5, MYCD, and/or phosphorylated AKT1. The expression cassettes maybe comprised in one or more replicable vectors, such as one or more viral vectors or one or more non-viral vectors. The one or more viral vectors may be one or more adeno-associated virus (AAV), non-integrated lentivirus, adenoviral vectors or retroviral vectors, and the one or more replicable vectors may be non-viral vectors and/or modified RNA delivery vectors, such as one or more non-viral vectors are disposed in a lipid delivery vehicle.

In embodiments, one, two, three, four, five, six or all seven of PHF7, TBX5, MEF2C, GATA4, HAND2, phosphorylated AKT1, and/or MYCD proteins or expression cassettes may be delivered 24 hours to one month following said MI. At least one of said PHF7, TBX5, and MEF2C proteins may be delivered multiple times, such as 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 times, or daily. In particular, PHF7, and/or PHF7 and TBX5, and optionally MEF2C, GATA4, HAND2, phosphorylated AKT1, and/or MYCD proteins are delivered multiple times, such as 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 times, or daily. In particular, at least one of said PHF7, TBX5, and optionally MEF2C, expression cassettes are delivered multiple times, such as 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 times, or daily. In particular, PHF7, and/or PHF7 and TBX5, and optionally MEF2C, GATA4, HAND2, phosphorylated AKT1, and/or MYCD expression cassettes are delivered multiple times, such as 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 times, or daily.

In yet another embodiment, there is provided a method preventing or delaying development or worsening of cardiac hypertrophy or heart failure in a subject having suffered a myocardial infarct (MI) comprising providing to said subject PHF7, and/or PHF7 and TBX5, and optionally further providing MEF2C, GATA4, HAND2, phosphorylated AKT1, and/or MYCD as proteins or as expression cassettes coding such proteins. The method may further comprise administering to said subject a secondary anti-hypertrophic or heart failure therapy, such as combinations of a PKD inhibitor, a beta blocker, an inotrope, a diuretic, ARNI, ACE-I, AII antagonist, BNP, a Ca⁺⁺-blocker, an SGLT2 inhibitor, a GLP-1 agonist, a neprilysin inhibitor, or an HDAC inhibitor. The method may prevent, delay, or reverse heart failure or cardiac hypertrophy. Delay comprises preventing or delaying cardiac hypertrophy, such as comprising preventing or delaying one or more of decreased exercise capacity, diastolic dysfunction, decreased cardiac ejection fraction, increased left ventricular end diastolic pressure, increased pulmonary capillary wedge pressure, decreased cardiac output or cardiac index, increased pulmonary artery pressures, increased left ventricular end systolic and/or diastolic dimensions, increased left and right ventricular wall stress, increased wall tension, decreased quality of life, and/or increased disease related morbidity or mortality. Reverse comprises improvement and/or increase in exercise capacity, increased cardiac ejection fraction, decreased left ventricular end diastolic pressure, decreased pulmonary capillary wedge pressure, increased cardiac output or cardiac index, decreased pulmonary artery pressures, decreased left ventricular end systolic and/or diastolic dimensions, decreased left and right ventricular wall stress, decreased wall tension, increased quality of life, and/or increased disease related morbidity or mortality as compared to measures prior to PHF7 and/or PHF7 cocktail administration.

In accordance with such methods, PHF7 proteins, and optionally PHF7 and TBX5, and/or optionally MEF2C, GATA4, HAND2, phosphorylated AKT1, and/or MYCD proteins may be administered to said subject, or PHF7 expression cassettes, and optionally TBX5, MEF2C, GATA4, HAND2, phosphorylated AKT1, and/or MYCD expression cassettes are administered to said subject. The method may further comprise administering an anti-inflammatory agent, small molecule, or micro-RNA to said subject. In accordance with such methods, at least one of said PHF7 and optionally TBX5, MEF2C, GATA4, HAND2, phosphorylated AKT1, and/or MYCD proteins or expression cassettes may be delivered multiple times, such as 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 times, or daily.

In additional embodiments, there are provided:
a method of improving exercise tolerance and/or reducing a decrease in exercise tolerance of a subject having suffered a myocardial infarction comprising delivering to said subject PHF7, and optionally TBX5, MEF2C, GATA4, HAND2, phosphorylated AKT1, and/or MYCD proteins or expression cassettes coding therefor;
a method of reducing incidence of hospitalization or hospital length of stay of a subject having suffered a myocardial infarction and/or heart failure comprising delivering to said subject PHF7, and optionally TBX5, MEF2C, GATA4, HAND2, phosphorylated AKT1, and/or MYCD proteins or expression cassettes coding therefor;
a method of improving cardiac function and/or preventing decrease in cardiac function of a subject having suffered a myocardial infarction comprising delivering to said subject PHF7, and optionally TBX5, MEF2C, GATA4, HAND2, phosphorylated AKT1, and/or MYCD proteins or expression cassettes coding therefor;
a method of improving quality of life of a subject comprising delivering to said subject PHF7, and optionally TBX5, MEF2C, GATA4, HAND2, phosphorylated AKT1, and/or MYCD proteins or expression cassettes coding therefor;
a method of decreasing morbidity of a subject having suffered a myocardial infarction comprising delivering to said subject PHF7, and optionally TBX5, MEF2C, GATA4, HAND2, phosphorylated AKT1, and/or MYCD proteins or expression cassettes coding therefor; and
a method of decreasing mortality of a subject having suffered a myocardial infarction comprising delivering to said subject PHF7, and optionally TBX5, MEF2C, GATA4, HAND2, phosphorylated AKT1, and/or MYCD proteins or expression cassettes coding therefor, such as further comprising administering and anti-inflammatory agent, small molecule, or micro-RNA to said subject.

Also provided is method of activating GATA4 and/or HAND2 in a cardiac cell comprising delivering to said cell PHF7, either by protein delivery or by delivery of an expression construct encoding PHF7.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-J. PHF7 promotes direct cardiac reprogramming. (FIG. 1A) Schematic. (FIG. 1B) Immunostaining of day 7 α-MHC-GFP transgenic TTF, CF, or MEF AGHMT±PHF7 iCLMs. α-MHC-GFP (green), cTnT (red), Hoechst (brightfield). Scale=2 mm. (FIG. 1C) Immunostaining of day 7 α-MHC-GFP transgenic TTF AGHMT±PHF7 iCLMs. α-MHC-GFP (green), cTnT (red), Hoechst (blue). Scale=100 μm. Biologically independent experiments were performed with similar results at least three times for (FIG. 1B) and (FIG. 1). (FIG. 1D) Immunostaining of day 21 human CF (NHCF-V) My-GHMT±PHF7 iCLMs and (FIG. 1E) quantification of % double positive cells (My-GHMT: MYOCD, GATA4, HAND2, MEF2C, and TBX5). cTnT (green), a-actinin (red), DAPI (blue). scale=200 μm. n=4 biologically independent samples, p****<0.0001. Data are presented as mean±SD values. (FIG. 1F) Representative flow cytometry plots of day 7 TTF iCLMs and (FIG. 1G) quantitative analysis of % double positive (α-MHC-GFP⁺/cTnT⁺) cells. n=3 biologically independent samples, p****<0.0001. Data are presented as mean±SD values. α-MHC-GFP (x-axis, FITC), cTnT (y-axis, 647). (FIG. 1H) Immunocytochemistry of day 1 AGHMT±PHF7 MEF iCLMs. α-MHC-GFP (green), Hoechst (blue), scale=200 μm. Biologically independent experiments were performed with similar results at least three times. (FIG. 1I) Number of Ca²⁺-fluxing cells per high powered field in day 14 AGHMT±PHF7 MEF iCLMs. n=3 biologically independent samples, p*=0.0135. Data are presented as mean±SD values. (FIG. 1J) Quantification of number of spontaneously beating cells per high power field in MEF iCLMs infected with Empty virus, PHF7, or AGHMT±PHF7. n=3 biologically independent samples, p****<0.0001, p***<0.001, p**<0.01. All statistical comparisons between groups were evaluated by one-way ANOVA analysis, with modification for multiple comparisons. Source data are provided as a Source data file.

FIGS. 2A-E. PHF7 globally activates the cardiac transcriptome. (FIG. 2A) Schematic. (FIG. 2B) Heatmap comparing differentially expressed transcripts in day 7 AGHMT±PHF7 TTF iCLMs identified by RNA-Seq. Upregulation (red), downregulation (blue). Color scale represents Z-score. n=3 biologically independent samples. (FDR≤0.1, FC≥2) (FIG. 2C) Fold change between AGHMT+PHF7 (6F) and AGHMT (5F) for selected cardiac markers and transcription factors. (FIGS. 2D-E) GO analysis showing biological processes associated with genes up-regulated (FIG. 2D) and down-regulated (FIG. 2E) by RNA-Seq.

FIGS. 3A-E. PHF7 reprograms cells to a cardiac fate in the absence of exogenous Gata4. (FIG. 3A) Immunocytochemistry of day 7 Empty, MT, PMT, and GMT-treated TTF iCLMs. α-MHC-GFP (green), cTnT (red), Hoechst (blue), scale=100 μm. Biologically independent experiments were performed with similar results at least three times. (FIG. 3B) Representative flow cytometry plots of day 7 TTF iCLMs treated with empty virus, GMT, or PMT. α-MHC-GFP (x-axis, FITC) and cTnT (y-axis, 647). (FIG. 3C) Quantitative analysis of % αMHC-GFP⁺ and % cTNT⁺ in day 7 TTF iCLMs by flow cytometry. n=3 biologically independent samples for Empty and GMT, n=4 biologically independent samples for PMT, p***=0.0001, p**=0.0083. Data are presented as mean±SD values. (FIG. 3D) RT-PCR analysis of Empty, MT, or PMT-infected day 7 TTF iCLMs (relative to Empty vector, normalized to GAPDH). n=3 biologically independent samples, p*=0.0189, p**=0.0078. Data are presented as mean±SD values. (FIG. 3E) RT-PCR analysis probing the 3′ UTR of Gata4 in Empty, MT, or PMT-infected day 7 TTF iCLMs. (relative to Empty vector, normalized to GAPDH). n=3 biologically independent replicates, p*=0.0187. Data are presented as mean±SD values. All statistical comparisons between groups were evaluated by one-way ANOVA analysis, with modification for multiple comparisons. Source data are provided as a Source data file.

FIGS. 4A-K. PHF7 binds to and activates cardiac enhancers (FIG. 4A) Schematic. (FIG. 4B) Heatmap of differential PHF7^{3xTy1} ChIP-Seq peaks in the presence or absence of AGHMT (FC>2) (±2 kb window centered on peak summit). (FIG. 4C) de novo PHF7^{3xTy1} motif analysis of reprogramming peaks by HOMER (AGHMT+PHF7^{3xTy1}-unique) (FDR threshold 10⁻³). (FIG. 4D) Heatmap ordered by 5F+PHF7^{3xTy1} peak signal aligned with H3K27ac, Gata4, Hand2, Mef2c, Tbx5 ChIP in day 2 AGHMT iCLMs (±2 kb window). (FIG. 4E) SE regions ranked and annotated by ROSE (n=1251) are plotted in increasing order based on their input-normalized H3K27ac ChIP-seq signal from P4 mouse ventricle. (FIG. 4F) Metagene plot of PHF7^{3xTy1} ChIP signal mapped to cardiac SEs with ±3 kb flanking region in the presence (green) or absence (orange) of AGHMT. (FIG. 4G) Metagene plot of H3K4me1, H3K4me2, H3K4me3, and H3K27ac ChIP signal derived from uninduced MEFs mapped to cardiac SEs with ±3 kb flanking region. (FIG. 4H) Genome browser shots of PHF7^{3xTy1}±AGHMT ChIP at the Myh6/Myh7 super-enhancer aligned with Gata4, Hand2, Mef2c^{3xTy1}, Tbx5^{3xTy1}, and H3K27ac ChIP in day 2 AGHMT iCLMs and P4 heart H3K27ac ChIP. (FIG. 4I) Immunostaining for Myh6^{enh}-hsp68-mCherry in Day 4 AGHMT+PHF7 MEF iCLMs. mCherry (red), Hoechst (blue), Scale=100 μm. Biologically independent experiments were performed with similar results at least three times. (FIG. 4J) FLAG co-immunoprecipitation assay in HEK293 cells transfected with GFP, PHF7^{FLAGHA}, and/or Gata4^{myc}±DSP crosslinker. (IP) Immunoprecipitation. (IB) Immunoblot. GAPDH is a loading control. Biologically independent experiments were performed with similar results three times. (FIG. 4K) Gata4 ChIP qPCR in day 2 iCLMs treated with AGHMT±PHF7. n=3 biologically independent samples. Gata4 TSS p*=0.0482. Myh6^{enh} p*=0.023. Tbx5 TSS p*=0.0197. Statistical analyses performed using unpaired two-tailed Student's t test. Data are presented as mean±SD values. (k) H3K27ac ChIP qPCR in day 2 iCLMs treated with Empty vector or AGHMT+PHF7. n=3 biologically independent samples. Gata4 TSS p***=0.001. Myh6^{enh} p**0.0043. Tbx5 TSS p****<0.0001. Data are presented as mean±SD values. Statistical analyses performed using one-way ANOVA analysis, with modification for multiple comparisons. Source data are provided as a Source data file.

FIGS. 5A-I. PHF7 interacts with SMARCD3/BAF60c to promote reprogramming (FIG. 5A) Heatmap demonstrating differential peaks by ATAC-Seq in day 2 AGHMT±PHF7 iCLMs (FC>2). (FIG. 5B) GO pathway analysis of regions with increased accessibility in the presence of PHF7 as determined by GREAT analysis. (FIG. 5) GO pathway analysis of regions with decreased accessibility in the presence of PHF7 as determined by GREAT analysis. (FIG. 5D) de novo motif analysis of regions with increased accessibility in the presence of PHF7 (FDR threshold 10⁻³). (FIG. 5E) FLAG co-immunoprecipitation in HEK293 cells transfected GFP, PHF7^{3xTy1}, and/or SMARCD3^{FLAG-HA}. (IP) Immunoprecipitation. (IB) Immunoblot. GAPDH is a loading control. (FIG. 5F) Schematic. (FIG. 5G) Immunocytochemistry of day 7α-MHC-GFP TTF iCLMs after infection with AGHMT±PHF7 and Scramble or shSmarcd3. α-MHC-GFP (green), Hoechst (blue), scale=200 μm. (FIG. 5H) Quantification of % α-MHC-GFP⁺, % cTnT⁺, and % double positive cells (n=4 biologically independent samples for % α-MHC-GFP⁺ samples and n=3 biologically independent samples for % cTnT⁺ and % double positive samples. p***<0.0001, p***<0.0004, p**<0.0013. Data are presented as mean±SD values. (FIG. 5I) Graphical model of PHF7 recognition of histone modification at cardiac super-enhancers and recruitment of Smarcd3/SWI/SNF complex and cardiac TFs to regulate cardiac gene transcription. All statistical comparisons between groups were evaluated by one-way ANOVA analysis, with modification for multiple comparisons. Source data are provided as a Source data file.

FIGS. 6A-G. (FIG. 6A) tSNE plots from single-cell RNA Seq analysis of Mesp1+ cardiac progenitors from E6.5-E7.5 demonstrate robust expression of Phf7 and Gata4 throughout the existence of these cells. (FIG. 6B) Western blot demonstrating increased cTnT and αMHC-GFP protein expression in day 7 AGHMT±PHF7 TTF iCLMs. Arrow indicates relevant cTnT band at 27 kD. GAPDH is a loading control. Biologically independent experiments were performed with similar results at least two times. (FIG. 6C) Representative FACs plot demonstrating side and forward scatter gating of live cells. (FIG. 6D) Quantitative analysis by flow cytometry demonstrates % αMHC-GFP⁺ and % cTNT⁺ cells in day 7 AGHMT±PHF7 TTF iCLMs. n=3 biologically independent samples. p****<0.0001. Data are presented as mean±SD values. (FIG. 6E) RT-PCR of TTF iCLMs 7 d after infection with AGHMT±PHF7 demonstrates increased expression of cardiac markers Myh6, Actc1, and Tnni3 in PHF7-treated cells. n=2 biologically independent samples. (FIG. 6F) Quantitative analysis of immunocytochemistry in Day 21 My-GHMT±PHF7 human CFs. n=4 biologically independent samples. p****<0.0001. Data are presented as mean±SD values. (FIG. 6G) RT-PCR of Day 21 My-GHMT±PHF7 human CF iCLMs for cardiac lineage markers. Relative to Empty virus control. Normalized to GAPDH. n=2 biologically independent samples. All statistical comparisons between groups were evaluated by one-way ANOVA analysis, with modification for multiple comparisons. Source data are provided as a Source data file.

FIGS. 7A-J. (FIG. 7A) Schematic for doxycycline-inducible expression strategy of PHF7 in reprogramming. (FIG. 7B) Schematic representation of doxycycline (dox) dosing strategy. Dox was administered for different intervals; 4 weeks on (D1 on), 6 days (D3 off), and 13 days (D10 off), immediately following infection of TTFs or MEFs with pRetroX-PHF7/pMXs-AGHMT/pMXs-PHF7. Cardiac reprogramming media was added at DO and changed along with dox every 2 days. (FIG. 7C) Western blot demonstrating activation of PHF7 expression in the presence of dox (100-1000 ng/mL), which is repressed in the absence of dox using the pRetroX-PHF7 system. GAPDH is a loading control. (FIG. 7D) qRT-PCR for PHF7 expression iCLM MEFs in the indicated samples after 28 days. n=3 biologically independent samples, p*=0.0468, p****<0.0001. Data are presented as mean±SD values. (FIG. 7E) qRT-PCR for Myh6 expression in iCLM TTFs in the indicated samples after 28 days. n=2 biologically independent samples. (FIG. 7F) Representative immunocytochemistry for αMHC-GFP (green) and cTNT (red) in indicated treatment groups for Day 28 TTF iCLMs and (FIG. 7G) quantification of % αMHC-GFP+, % cTnT+, and % α-actinin+ cells in indicated treatment groups Hoechst (blue). Scale=100 μm. n=3 biologically independent samples. % αMHC-GFP: D1 on p***=0.0001, D3 off p****<00.0001, D10 off p***=0.00007. % cTnT+: D1 on p*=0.0117, D3 off p**=0.0015, D10 off p***=00.001. % α-actinin+: D1 on p***=0.001, D3 off p***=0.0006, D10 off p***=0.0003. Data are presented as mean±SD values. (FIG. 7H) Immunocytochemistry for α-actinin in D3 off TTF iCLMs at day 28. Zoomed view demonstrates sarcomeric organization. Scale=100 μm. Biologically independent experiments were performed with similar results at least three times. (FIG. 7I) Quantification of number of spontaneously beating cells per high power field in MEF iCLMs. n=3 biologically independent experiments. Day 7: D1 on p***0.0007, D3 off p****<00.0001, Day 10 off p**=00.0028. Day 10: D1 on p**=0.0071, D3 off p***=0.0009, D10 off p**=0.0041. Day 21: D1 on p***=0.0006, D3 off p****<0.0001, D10 off p***=0.002. (FIG. 7J) Quantification of intracellular calcium flux by Fluo-4 assay in day 28 iCLM MEFs. n=3 biologically independent samples. p****<0.0001. Data are presented as mean±SD values. All statistical comparisons between groups were evaluated by one-way ANOVA analysis, with modification for multiple comparisons. Source data are provided as a Source data file.

FIGS. 8A-I. (FIG. 8A) Unbiased heatmap displaying the 50 most upregulated transcripts by RNA-Seq in the presence of AGHMT+PHF7. (FIG. 8B) Unbiased heatmap displaying the 50 most downregulated transcripts by RNA-Seq in the presence of AGHMT+PHF7. Color scale by Z score. (FIG. 8C) Plots of cardiac reprogramming transcription factors upregulated in the presence of PHF7 by RNA-Seq analyses (normalized counts per million). n=3 biologically independent replicates. p****<0.0001. Data are presented as mean±SD values. (FIG. 8D) Western blot of lysates from day 7 TTF iCLMs following GHMT and AGHMT reprogramming ±PHF7 blotting for Gata4 and Mef2c expression. GAPDH is a loading control. (FIG. 8E) RNA-seq tracks at Gata4 3′ UTR and Tbx5 3′UTR from Empty, AGHMT (5F), and AGHMT+PHF7 (5F+PHF7) samples. (ED FIGS. 3F-G) Enrichment plots of indicated gene sets and their nominal p-value of genes upregulated by PHF7 (FIG. 8F) and downregulated by PHF7 (FIG. 8G). (FIG. 8H) EdU pulse labeling of day 1 TTF iCLMs in the presence of Empty, PHF7, or AGHMT+PHF7. EdU (magenta), Hoechst (blue). Scale=100 μm. (FIG. 8I) Quantification of EdU pulse labeling of day 1 TTF iCLMs in the presence of Empty, PHF7, or AGHMT+PHF7. n=3 biologically independent samples. Empty v AGHMT: p***0.0008. AGHMT±PHF7: ns=0.9993. Data are presented as mean±SD values. All statistical comparisons between groups were evaluated by one-way ANOVA analysis, with modification for multiple comparisons. Source data are provided as a Source data file.

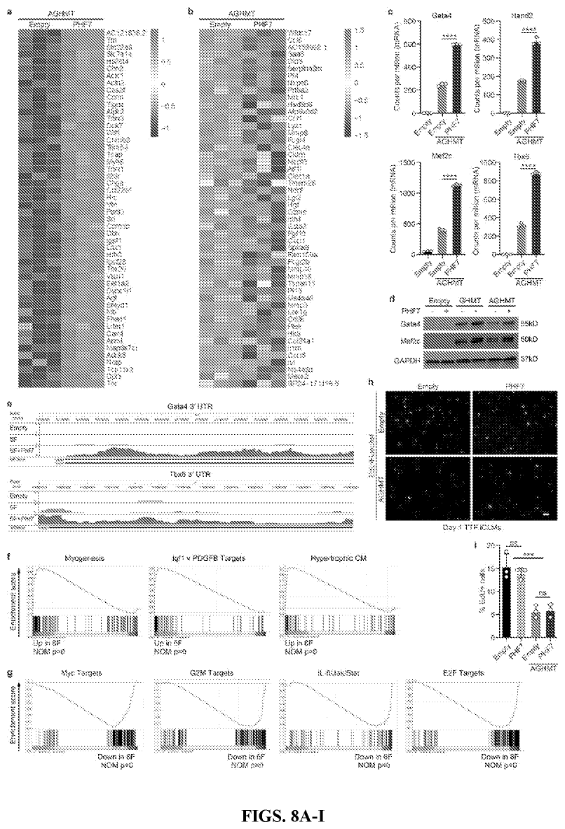

FIGS. 9A-C. (FIG. 9A) Representative immunocytochemistry images demonstrate that PHF7 augments GMT, GHMT, or AGHMT reprogramming in both αMHC-GFP transgenic TTF and MEF iCLMs at day 7. αMHC-GFP (green), scale=200 μm. Biologically independent experiments were performed with similar results at least three times. (FIG. 9B) Quantitative analysis of % of αMHC-GFP⁺, cTnT⁺, and double positive TTF iCLMs 7 d after infection with MT, GMT, GHMT, or AGHMT±PHF7. n=2 biologically independent samples. (FIG. 9C) Quantification number of cells with active calcium flux per high-powered field by Fluo-4 assay in Day 14 iCLM MEFs infected with GMT, GHMT, or AGHMT±PHF7. n=3 biologically independent samples, p***=0.0003. Data are presented as mean±SD values. All statistical comparisons between groups were evaluated by one-way ANOVA analysis, with modification for multiple comparisons. Source data are provided as a Source data file.

FIGS. 10A-I. (FIG. 10A) Western blot for PHF7 of lysates from HEK293 cells transfected with PHF7, N-terminal 3x-Ty1-PHF7, or C-terminal PHF7-3xTy1. GAPDH is a loading control. (FIG. 10B) Representative immunocytochemistry images demonstrate that PHF^{3xTy1} augments reprogramming to a similar extent as untagged PHF7 in day 7 MEF iCLMs. Scale=200 μm. Biologically independent experiments were performed with similar results at least three times. (FIG. 10C) Genomic location of annotated PHF7^{3xTy1} peaks in the presence of reprogramming factors (AGHMT±PHF7^{3xTy1}-unique). (FIG. 10D) Genomic location of annotated PHF7^{3xTy1} peaks in the absence of reprogramming factors (PHF7^{3xTy1}-unique). (FIG. 10E) GO enrichment analysis of PHF7^{3xTy1} peaks in the absence of AGHMT (PHF7^{3xTy1}-unique) as determined by GREAT analysis. (FIG. 10F) GO enrichment analysis of PHF7^{3xTy1} peaks in the presence of AGHMT (AGHMT±PHF7^{3xTy1}-unique) as determined by GREAT analysis. (FIG. 10G) de novo motif analysis of PHF7^{3xTy1} peaks in the absence of AGHMT by HOMER (PHF7^{3xTy1}-unique). (FDR threshold 10⁻³). (FIG. 10H) Motif frequency analysis of Gata4, Mef2c, Hand2, Tbx5, TEAD, and CTCF centered on PHF7 peak signal in reprogramming (AGHMT+PHF7^{3xTy1})-unique peaks (red), unchanged PHF7^{3xTy1} peaks (gray), or PHF7^{3xTy1}-unique peaks in the absence of reprogramming factors (green). (FIG. 10I) Heatmap ordered by PHF7^{3xTy1} signal in absence of AGHMT (PHF7^{3xTy1}-unique peaks) aligned with H3K27ac, Gata4, Hand2, Mef2c, Tbx5 ChIP signal from day 2 AGHMT iCLMs. Normalized ChIP-seq signal with ±2 kb window centered around peak summit and sorted in descending order by signal intensity. Source data are provided as a Source data file.

FIGS. 11A-B. (FIG. 11A) Heatmap ordered by PHF7^{3xTy1} peaks aligned with H3K4me1, H3K4me2, H3K4me3, H3K27ac, H3K79me2, and H3K27me3 histone ChIP from uninduced MEFs. Normalized ChIP-seq signal with ±2 kb window centered around peak summit and sorted in descending order by signal intensity. (FIG. 11B) Heatmap ordered by PHF7^{3xTy1} peaks bound to P4 heart enhancers aligned with H3K4me1, H3K4me2, H3K4me3, H3K27ac, H3K79me2, and H3K27me3 histone ChIP from uninduced MEFs. Normalized ChIP-seq signal with ±2 kb window centered around peak summit and sorted in descending order by signal intensity.

FIGS. 12A-C. (FIG. 12A) Schematic of enhancer-hsp68-mCherry retroviral generation and application to AGHMT±PHF7 MEF iCLM reprogramming. (FIG. 12B) Representative images of Myh6^{enh}, Phf7^{enh}, Thra^{enh}, Tns1^{enh}, and Tbx20^{enh}-hsp68-mCherry activation in Day 4 AGHMT±PHF7 MEF iCLMs. mCherry (red), Hoechst (blue). Scale=100 μm. Biologically independent experiments were performed with similar results at least three times. (FIG. 12C) Quantification of enhancer activation by % mCherry+ cells in Day 4 AGHMT±PHF7 MEF iCLMs treated with respective enhancer-hsp68-mCherry constructs. n=3 biologically independent samples. Myh6^{enh}: p***=0.0006, Phf7^{enh}: p****<0.0001, Thra^{enh}: p***=0.0001, Tns1^{enh}: p**=0.0023, Tbx20^{enh}: p***=0.0008. Data are presented as mean±SD values. All statistical comparisons between groups were evaluated by one-way ANOVA analysis, with modification for multiple comparisons. Source data are provided as a Source data file.

FIGS. 13A-J. (FIGS. 13A-F) Genome browser shots of PHF7^{3xTy1} binding ±AGHMT at the (FIG. 13A) Tnni1/Tnnt2, (FIG. 13B) Gata4, (FIG. 13C) Hand2, (FIG. 13D) Mej2c, (FIG. 13E) Tbx5, and (FIG. 13F) Phf7 super-enhancer loci, aligned with binding profiles of cardiac TFs by Gata4, Hand2, Mef2c, Tbx5, and H3K27ac ChIP in day 2 AGHMT iCLMs. (FIG. 13G) Genome browser shot of P4 mouse atrium and ventricle TF (Gata4, Nkx2-5, and Tbx5) and H3K27ac ChIP alignment at the Phf7 locus. (FIG. 13H) FLAG co-immunoprecipitation in HEK293 cells transfected with PHF7^{FLAG} and GFP, Gata4^{myc}, Hand2^{myc}, or Mef2c^{myc}. (IP) Immunoprecipitation. (IB) Immunoblot. GAPDH is a loading control. Biologically independent experiments were performed with similar results at least three times. (FIG. 13I) PHF7^{3xTy1} ChIP qPCR in day 2 iCLMs at the Gata4 locus treated with PMT as compared to IgG control. n=3 biologically independent samples, p*=0.0261, by unpaired two-tailed Student's t-test. Data are presented as mean±SEM values. Mef2c^{3xTy1} ChIP qPCR at the Gata4 locus in day 2 iCLMs treated with Mef2c^{3xTy1}+Tbx5 (MT) or PHF7+Mef2c^{3xTy1}+Tbx5 (PMT) as compared to IgG control. n=3 biologically independent replicates. p*=0.0250, by one-way ANOVA with adjustment for multiple comparisons. Data are presented as mean±SD values. H3K27ac ChIP qPCR at the Gata4 locus in day 2 iCLMs treated with Empty vector, MT, or PMT cocktail. n=3 biologically independent samples. p*=0.0320. Data are presented as mean±SD values. (FIG. 13J) PHF7^{3xTy1} ChIP qPCR in day 2 iCLMs at the Myh6 locus treated with PMT as compared to IgG control. n=3 biologically independent samples. p*=0.0184, as determined by unpaired two-tailed Student's t test. Mef2c^{3xTy1} ChIP qPCR at the Myh6 locus in day 2 iCLMs treated with Mef2c^{3xTy1}+Tbx5 (MT) or PHF7+Mef2c^{3xTy1}+Tbx5 (PMT) as compared to IgG control. n=3 biologically independent samples. p*=0.0118, as determined by one-way ANOVA with adjustment for multiple comparisons. H3K27ac ChIP qPCR at the Myh6 locus in day 2 iCLMs treated with Empty vector, MT, or PMT cocktails. n=3 biologically independent samples. p*=0.0202, as determined by one-way ANOVA with adjustment for multiple comparisons. Data are presented as mean±SD values. Source data are provided as a Source data file.

FIGS. 14A-F. (FIG. 14A) Table with top shared proteomics hits ranked based on fold change relative to control and normalized abundance from miniTurbo proximity biotinylation assay performed in PHF7^{miniTubo} and AGHMT±PHF7^{miniTurbo} infected MEFs. (FIG. 14B) Heatmap demonstrating AGHMT±PHF7^{miniTurbo}-unique proteins. (related to Supplementary Data Table 2). Biologically independent experiments for AGHMT±PHF7^{miniTurbo} samples were performed with similar results two times. (FIG. 14C) GO Pathway analysis of unique and shared proteins between PHF7^{miniTurbo} and AGHMT±PHF7^{miniTurbo} biotin-treated samples. (FIG. 14D) Streptavidin IP of AGHMT(5F)+PHF7^{miniTurbo} infected MEFs in biotin-treated and untreated control followed by western blot. (IP) Immunoprecipitation. (IB) Immunoblot. Biologically independent experiments were performed with similar results two times. (FIG. 14E) Immunocytochemistry images demonstrate overexpression of pMXs-SMARCD3 with AGHMT or AGHMT±PHF7 does not augment reprogramming in day 7 TTF iCLMs. αMHC-GFP (green), Hoechst (blue). (FIG. 14F) Realtime PCR validation of Smarcd3 transcript knockdown by shSmarcd3 in murine neuro2a (N2a). n=2 biologically independent samples. Source data are provided as a Source data file.

FIGS. 15A-D. (FIG. 15A) Immunocytochemistry of day 7 PHF7, TBX5, PT, and PMT-treated TTF iCLMs. α-MHC-GFP (green), Hoechst (blue). Biologically independent experiments were performed with similar results at least three times. (FIG. 15B) Quantitative analysis of % αMHC-GFP⁺ cells in day 7 TTF iCLMs. n=3 biologically independent samples, p***=0.0001, p**=0.0083. Data are presented as mean±SD values. (FIG. 15C) Representative flow cytometry plots of day 7 TTF iCLMs treated with empty virus, PT, or PMT. α-MHC-GFP (x-axis, FITC) and cTnT (y-axis, 647). (FIG. 15D) RT-PCR analysis of Empty, Tbx5 (T), PT, Mef2c and Tbx5 (MT), or PMT-infected day 7 TTF iCLMs (relative to Empty vector, normalized to GAPDH). n=3 biologically independent samples. Data are presented as mean±SD values. All statistical comparisons between groups were evaluated by one-way ANOVA analysis, with modification for multiple comparisons. Source data are provided as a Source data file.

FIGS. 16A-H. PHF7 activates reprogramming with TBX5 alone. (FIG. 16A) Schematic. (FIG. 16B) Immunocytochemistry of day 7 TBX5, PT, MEF2C+TBX5, and PMT-treated TTF iCLMs. α-MHC-GFP (green), Hoechst (blue). Biologically independent experiments were performed with similar results at least three times. (FIG. 16C) Representative flow cytometry plots of day 7 TTF iCLMs treated with empty virus, Empty, PT, or PMT. α-MHC-GFP (x-axis, FITC) and cTnT (y-axis, 647). (FIG. 16D) RT-PCR analysis of Empty, Tbx5 (T), PT, Mef2c and Tbx5 (MT), or PMT-infected day 7 TTF iCLMs (relative to Empty vector, normalized to GAPDH). n=3 biologically independent samples. Data are presented as mean±SD values. (FIG. 16E, 16F) Immunostaining of day 21 adult human CF (NHCF-V) PT or PMT±Myocd iCLMs, cTnT (green), α-actinin (red), DAPI (blue). (FIGS. 16G-H). RT-PCR of Day 21 GFP, PHF7±Myocd, PT±Myocd, and PMT±Myocd-treated adult human CF iCLMs for cardiac lineage markers. Relative to GFP virus control. Normalized to GAPDH. n=2 biologically independent samples. All statistical comparisons between groups were evaluated by one-way ANOVA analysis, with modification for multiple comparisons. Source data are provided as a Source data file.

FIGS. 17A-G. PHF7 cocktails activate a global but distinct cardiac transcriptome. (FIG. 17A) Schematic. (FIG. 17B) Heatmap comparing differentially expressed transcripts in day 7 Empty, PHF7 (P), TBX5 (T), PT, PMT, and GMT-treated TTF iCLMs identified by RNA-Seq. Upregulation (red), downregulation (blue). Color scale represents Z-score. n=3 biologically independent samples. (FDR≤0.1, FC≥2) (FIG. 17C) Volcano map comparing differentially expressed genes between GMT (blue) and PMT (red) treated iCLMs. (FIG. 17D). Differential fold change expression of selected cardiac markers and transcription factors upregulated in the PMT condition as compared to GMT. (FIGS. 17E-F) GO analysis showing biological processes associated with genes differentially upregulated by PMT (FIG. 17E) or by GMT (FIG. 17F) as determined by RNA-Seq. (FIG. 17G). Heatmap clustered by maximum gene unique expression of Empty, PHF7 (P), TBX5 (T), PT, PMT, or GMT-treatment groups as identified by RNA-Seq.

FIGS. 18A-J. PHF7 cocktails improve cardiac function following myocardial infarction. (FIG. 18A). Schematic describing permanent LAD ligation, retroviral delivery, and functional assessment. (FIG. 18B) Ejection fraction (%) and (FIG. 18C) Fractional shortening (%) of mice subjected to LAD ligation followed by intramyocardial injection of GFP, PT, PMT, GMT, PGMT, as well as Sham (no MI) control with PBS injection was evaluated at 24 h, 7d, and 21 d post-MI by echocardiography (n=8-9 mice per group, data presented as mean±SD. (FIG. 18D) Quantification of percent (%) change in ejection fraction and (FIG. 18E) fractional shortening at 7 d and 21 d post-MI, as compared to 24 h post-MI (n=8-9 mice per group, data presented as mean±SEM. (FIG. 18F) Bar graph representation of ejection fraction (%) at 24 h, 7 d, and 21 d post-MI (n=8-9 mice per group, data presented as mean±SEM. (FIG. 18G) Representative echocardiography m-mode images at 21 d post-MI in GFP, PT, PMT, GMT, PGMT-treated animals; arrows indicate the infarcted anterior wall. (FIG. 18H). Cardiac MRI was performed on surgical groups at 14-16 weeks post-MI and End Diastolic Volume (mL), End Systolic Volume (mL), and (FIG. 18I) Ejection fraction (%) were calculated (n=3-5 mice per group, data presented as mean±SD. (FIG. 18J) Representative short axis T2-weighted MRI images of Sham, GFP, PT, PMT, GMT, and PGMT at end diastole.

FIGS. 19A-E. PHF7 decreases fibrotic scar and induces remuscularization following myocardial infarction. (FIG. 19A). Comparison of cardiac fibrosis across treatment groups in GFP, PT, PMT, GMT, and PGMT treated hearts by Masson's Trichrome staining at 16 weeks post-MI. Cardiac fibrosis was evaluated at 500 μm intervals across 5 levels (L1-L5), with L1 beginning at the last normoxic section prior to suture placement. Mouse ID number is indicated in the lower right of each series. Scale bar=1000 μm. (FIGS. 19B-C) Higher magnification of the ischemic region in GFP, PT, PMT, GMT, and PGMT mice indicates remuscularization of the scarred region in (FIG. 19B) Masson's trichrome and (FIG. 19C) troponin-stained sections (troponin=green, hoescht=blue, scale=100 μm). (FIG. 19D) Comparison of cardiac fibrosis across treatment group in GFP, PT, PMT, GMT, and PGMT treated hearts by Picosiurius Red staining. Representative images from n=2 hearts per group at L4 are shown. Scale bar=1000 μm (FIG. 19E) Quantification of fibrotic area in heart sections stained with picosirius red where fibrotic area is the sum of fibrotic area at L2 through L5 (n=8-9 hearts/group).

FIGS. 20A-D. PHF7 induces direct reprogramming of fibroblasts to cardiomyocytes following myocardial infarction. (FIG. 20A). Schematic describing generation of Postn^{MCM/+}/Rosa26^{rdTO/+} mice for lineage tracing. (FIG. 20B). Schematic of lineage tracing strategy for tamoxifen (TMX) delivery and permanent LAD ligation. (FIG. 20C) Whole mount fluorescence demonstrating tdTO expression specific to the infarcted region in Postn^{MCM/+}/Rosa26^{rdTO/+}21 days following MI. (FIG. 20D) Immunohistochemistry of sections of Postn^{MCM/+}/Rosa26^{rdTO/+} mice injected with GFP, PT, or PMT retroviral cocktail post-MI and exposed to TMX chow for 6 weeks. cTnT (green), tdTomato/DSRed (red), DAPI (blue).

FIGS. 21A-K. (FIG. 21A) Ejection fraction (%) and © Fractional shortening (%) of mice subjected to LAD ligation followed by intramyocardial injection of GFP or PHF7 as well as Sham (no MI) control with PBS injection was evaluated at 24 h, 7d, and 21 d post-MI by echocardiography (n=8-9 mice per group, data presented as mean±SD. (FIG. 21B) Quantification of percent (%) change in ejection fraction and fractional shortening at 7 d and 21 d post-MI, as compared to 24 h post-MI (n=8-9 mice per group, data presented as mean±SEM. (FIG. 21C) Representative echocardiography m-mode images at 21 d post-MI in GFP and PHF7-treated animals. (FIG. 21D) Comparison of cardiac fibrosis between GFP and PHF7-treated hearts by Masson's Trichrome staining at 16 weeks post-MI. Cardiac fibrosis was evaluated at 500 μm intervals across 5 levels (L1-L5), L1 beginning at the last normoxic section prior to suture placement. L4 is pictured for all samples. (FIG. 21E) Comparison of cardiac fibrosis between GFP and PHF7-treated hearts by Picosiurius Red staining. Representative images at L4 are shown. (FIG. 21F) Immunohistochemistry of sections of Postn^{MCM/+}/Rosa26^{rdTO/+} mice injected with PHF7 retroviral cocktail post-MI and exposed to TMX chow for 6 weeks. cTnT (green), tdTomato/DSRed (red), DAPI (blue). (FIG. 21G) Immunostaining of day 21 adult human CF (NHCF-V) PHF7±Myocd iCLMs, cTnT (green), α-actinin (red), DAPI (blue). (FIG. 21H). RT-PCR of Day 21 GFP, PHF7, and PHF7+Myocd adult human CF iCLMs for cardiac lineage markers. Relative to GFP virus control. Normalized to GAPDH. n=2 biologically independent samples. (FIG. 21I) Heatmap comparing differentially expressed transcripts in Empty versus PHF7-treated day 7 TTF iCLMs identified by RNA-Seq (FDR<0.1, FC>2). Upregulation (red), downregulation (blue). n=3 biological replicates (FIG. 21J) Base mean of copies/million of selected cardiac transcription factors in Empty vs PHF7-treated day 7 TTF iCLMs identified by RNA-Seq (n=3 biological replicates). (FIG. 21K) Base mean of copies/million of selected cardiac transcription factors in Empty, PHF7, Tbx5, PT, MT, PMT, ±GMT-treated day 7 TTF iCLMs identified by RNA-Seq (n=3 biological replicates).

FIG. 22. PHF7 as a single factor and within minimalist transcription factor cocktails improve cardiac function post-MI via direct fibroblast to cardiomyocyte reprogramming.

### DETAILED DESCRIPTION OF ILLUSTRATIVE EMBODIMENTS

Cardiovascular diseases are the leading cause of death in the US and worldwide, claiming 18 million lives annually. Ischemic heart disease is dominant amongst these, precipitating heart failure, an incurable, costly, and deadly disease. Heart failure impacts 38 million people worldwide and represents the single most significant financial burden to health care systems, costing the United States alone $30 billion annually. This is due to the fact that the adult human heart possesses minimal regenerative capacity. Heart failure is commonly caused by myocardial infarction (MI). During myocardial infarction, the heart cells, cardiomyocytes (CMs), undergo massive cell death, and through activation of resident cardiac fibroblasts (CFs), they are replaced by scar tissue. Scar tissue is not contractile, fails to contribute to cardiac function, and often plays a detrimental role in heart function. Ultimately, this pathogenic injury response progresses to chronic heart failure and death.

Despite advances in pharmacotherapies, percutaneous interventions, and invasive mechanical support devices, disease burden and mortality remain astronomical, with heart failure bearing a 50% 5-year mortality—a statistic that rivals even the most aggressive neoplasms. Currently, the only cure for heart failure is transplantation. Donor hearts for transplantation are a severely limited resource; ˜2,000 heart transplants occur annually despite the millions of patients with end-stage heart failure patients who could benefit from this curative therapy. Thus, new therapies and curative approaches for heart failure are desperately needed.

While treatment with pharmacological agents represents the primary mechanism for reducing or eliminating the manifestations of heart failure, the impact of pharmacotherapies are generally modest and mortality remains astronomical despite adherence to guideline-directed medical therapy. Diuretics manage symptoms of heart failure but have never been proven to improve mortality. First line agents for heart failure include beta blockers (e.g., carvedilol and metoprolol), angiotensin receptor-neprolysin inhibitor (ARNi) (e.g., sacubitril-valsartan), and/or angiotensin converting enzyme (ACE) inhibitors (e.g., enalopril and lisinopril) which may provide symptomatic relief, and have also been reported to decrease mortality (Young et al., 1989). However, the beta blockers have a multitude of side effects including bradycardia and fatigue and ARNis and ACE inhibitors are associated with adverse effects and are contraindicated in patients with certain disease states (e.g., hypotension, acute kidney injury, hyperkalemia, angioedema, renal artery stenosis). Further, many heart failure patients are unable to tolerate these medications due to low blood pressures and heart rates. Similarly, inotropic agent therapy (i.e., a drug that improves cardiac output by increasing the force of myocardial muscle contraction) although often necessary as a bridge to transplant, is associated with a panoply of adverse reactions, including accelerating mortality from heart failure itself.

Given the inability of CMs to proliferate and reconstitute functional myocardium following injury, numerous cellular approaches have been attempted at both the bench and bedside. These approaches have focused on delivery of pluripotent and somatic stem cells to the injured myocardium, the results of which have been highly variable at best. Alternative approaches to induce repair have focused on harnessing the cells resident in the human heart to induce remuscularization following injury through forced cell fate conversion of resident fibroblasts into functional CMs, in a process termed direct cardiac reprogramming, which is the focus of this disclosure.

These and other aspects of the disclosure are described in detail below.

### I. REPROGRAMMING FACTORS

Reprogramming factors are proteins, micro-RNAs, or small molecules that have been demonstrated to facilitate directed cell fate conversion. Reprogramming factors considered in this disclosure include transcription factors. A transcription factor (sometimes called a sequence-specific DNA-binding factor) is a protein that binds to specific DNA sequences, thereby controlling the movement (or transcription) of genetic information from DNA to mRNA. Transcription factors perform this function alone or with other proteins in a complex, by promoting (as an activator), or blocking (as a repressor) the recruitment of RNA polymerase (the enzyme that performs the transcription of genetic information from DNA to RNA) to specific genes.

A defining feature of transcription factors is that they contain one or more DNA-binding domains (DBDs), which attach to specific sequences of DNA adjacent to the genes that they regulate. Other reprogramming factors considered in this disclosure include proteins such as coactivators, chromatin remodelers, histone readers, histone acetylases, deacetylases, kinases, and methylases. While the aforementioned proteins also play crucial roles in gene regulation, they lack DNA-binding domains, and, therefore, are not classified as transcription factors. Furthermore, several micro-RNAs, which are a class of small non-coding RNAs that control gene expression, and small molecules that impact important signaling cascades have been shown to play critical roles in cardiac reprogramming and are considered in this disclosure. The present disclosure involves the inventors' observation that certain reprogramming factors can combine to efficiently reprogram adult cardiac fibroblasts into cardiomyocytes, which is presently a highly inefficient process in adult human and mouse fibroblasts. In particular, it is shown that addition of PHF7 to currently utilized reprogramming cocktails comprised of combinations of phosphorylated AKT1, GATA4, HAND2, MEF2C, and/or TBX5 markedly increases reprogramming efficiency. Further, PHF7 is able to increase reprogramming efficiency in the presence of minimal factors, with a combination of PHF7+TBX5 alone, and optionally further including MEF2C, constituting a powerful cardiac reprogramming cocktail of genes.

### A. PHF7

PHD finger protein 7, or PHF7, is a protein that in humans is encoded by the PHF7 gene.

This gene is expressed in the testis in Sertoli cells but not germ cells. The protein encoded by this gene contains plant homeodomain (PHD) finger domains, also known as leukemia-associated protein (LAP) domains, believed to be involved in transcriptional regulation. The protein, which localizes to the nucleus of transfected cells, has been implicated in the transcriptional regulation of spermatogenesis. Importantly, this protein is further has been described as a histone reader.

Diseases associated with PHF7 include Granulomatous Orchitis and Polyposis Syndrome, Hereditary Mixed, 1. An important paralog of this gene is G2E3. Alternate splicing results in multiple transcript variants of this gene.

A representative mRNA for PH7 is NM_016483.7 (SEQ ID NO: 1) and protein is NP_057567.3 (SEQ ID NO: 2).

### B. TBX5

T-box transcription factor TBX5 is a protein that in humans is encoded by the TBX5 gene. This gene is a member of a phylogenetically conserved family of genes that share a common DNA-binding domain, the T-box. T-box genes encode transcription factors involved in the regulation of developmental processes. This gene is closely linked to related family member T-box 3 (ulnar mammary syndrome) on human chromosome 12. The encoded protein may play a role in heart development and specification of limb identity. Mutations in this gene have been associated with Holt-Oram syndrome, a developmental disorder affecting the heart and upper limbs. Several transcript variants encoding different isoforms have been described for this gene. See Basson et al. (1997) and Terrett et al. (1994).

TBX5 (T-box 5); mRNA=NM_000192.3 (SEQ ID NO: 3); Protein=NP_000183.2 (SEQ ID NO: 4).

### C. MEF2C

Myocyte-specific enhancer factor 2C also known as MADS box transcription enhancer factor 2, polypeptide C is a protein that in humans is encoded by the MEF2C gene. MEF2C is a transcription factor in the MEF2 family. The gene is located at 5q14.3 on the minus strand and is 200,723 bases in length. The encoded protein has 473 amino acids with a predicted molecular weight of 51.221 kD. Three isoforms have been identified. Several post translational modifications have been identified including phosphorylation on serine-59 and serine-396, sumoylation on lysine-391, acetylation on lysine-4 and proteolytic cleavage. The mature protein is found in the nucleus and the gene's expression is maximal in the post natal period.

MEF2C has been shown to interact with MAPK7, EP300, Sp1 transcription factor, TEAD1, SOX18, HDAC4, HDAC7 and HDAC9. This gene is involved in cardiac morphogenesis and myogenesis and vascular development. It may also be involved in neurogenesis and in the development of cortical architecture. Mice without a functional copy of the Mej2c gene die before birth and have abnormalities in the heart and vascular system. In human mutations of this gene have resulted in severe psychomotor retardation, periodic tremor and an abnormal motor pattern with mirror movement of the upper limbs observed during infancy, hypotonia, abnormal EEG, epilepsy, absence of speech, autistic behavior, bruxism, and mild dysmorphic features, mild thinning of the corpus callosum and delay of white matter myelination in the occipital lobes. See McDermott et al. (1993) and Molkentin et al. (1996).

MEF2C (myocyte enhancer factor 2C); mRNA=NM_002397.5 (SEQ ID NO: 5); Protein=NP_002388.2 (SEQ ID NO: 6).

### D. GATA4

GATA4 (GATA binding protein 4) encodes a member of the GATA family of zinc-finger transcription factors. Members of this family recognize the GATA motif which is present in the promoters of many genes. This protein is thought to regulate genes involved in embryogenesis and in myocardial differentiation and function and is necessary for normal testicular development. Mutations in this gene have been associated with cardiac septal defects. Additionally, alterations in gene expression have been associated with several cancer types. Alternative splicing results in multiple transcript variants.

A representative GATA4 mRNA is NM_001308093.3 (SEQ ID NO: 7) and protein is NP_001295022.1 (SEQ ID NO: 8).

### E. HAND2

HAND2 (heart and neural crest derivatives expressed 2) is belongs to the basic helix-loop-helix family of transcription factors. This gene product is one of two closely related family members, the HAND proteins, which are asymmetrically expressed in the developing ventricular chambers and play an essential role in cardiac morphogenesis. Working in a complementary fashion, they function in the formation of the right ventricle and aortic arch arteries, implicating them as mediators of congenital heart disease. In addition, this transcription factor plays an important role in limb and branchial arch development.

A representative HAND2 mRNA is NM_021973.3 (SEQ ID NO: 9) and protein is NP_068808.1 (SEQ ID NO: 10).

### F. Myocardin

MYCD (myocardin) is a nuclear protein, which is expressed in heart, aorta, and in smooth muscle cell-containing tissues. It functions as a transcriptional co-activator of serum response factor (SRF) and modulates expression of cardiac and smooth muscle-specific SRF-target genes, and thus may play a crucial role in cardiogenesis and differentiation of the smooth muscle cell lineage. Alternatively spliced transcript variants encoding different isoforms have been found for this gene. [provided by RefSeq, September 2009]

A representative MYCD mRNA is NM_001146312.3 (SEQ ID NO: 11) and protein is NP_001139784.1 (SEQ ID NO: 12).

### G. AKT1

The AKT1 (AKT serine/threonine kinase 1) gene encodes one of the three members of the human AKT serine-threonine protein kinase family which are often referred to as protein kinase B alpha, beta, and gamma. These highly similar AKT proteins all have an N-terminal pleckstrin homology domain, a serine/threonine-specific kinase domain and a C-terminal regulatory domain. These proteins are phosphorylated by phosphoinositide 3-kinase (PI3K). AKT/PI3K forms a key component of many signalling pathways that involve the binding of membrane-bound ligands such as receptor tyrosine kinases, G-protein coupled receptors, and integrin-linked kinase. These AKT proteins therefore regulate a wide variety of cellular functions including cell proliferation, survival, metabolism, and angiogenesis in both normal and malignant cells. AKT proteins are recruited to the cell membrane by phosphatidylinositol 3,4,5-trisphosphate (PIP3) after phosphorylation of phosphatidylinositol 4,5-bisphosphate (PIP2) by PI3K. Subsequent phosphorylation of both threonine residue 308 and serine residue 473 is required for full activation of the AKT1 protein encoded by this gene. Phosphorylation of additional residues also occurs, for example, in response to insulin growth factor-1 and epidermal growth factor. Protein phosphatases act as negative regulators of AKT proteins by dephosphorylating AKT or PIP3. The PI3K/AKT signalling pathway is crucial for tumor cell survival. Survival factors can suppress apoptosis in a transcription-independent manner by activating AKT1 which then phosphorylates and inactivates components of the apoptotic machinery. AKT proteins also participate in the mammalian target of rapamycin (mTOR) signalling pathway which controls the assembly of the eukaryotic translation initiation factor 4F (eIF4E) complex and this pathway, in addition to responding to extracellular signals from growth factors and cytokines, is disregulated in many cancers. Mutations in this gene are associated with multiple types of cancer and excessive tissue growth including Proteus syndrome and Cowden syndrome 6, and breast, colorectal, and ovarian cancers. Multiple alternatively spliced transcript variants have been found for this gene. Of note this protein is constitutively active in the phosphorylated state.

A representative AKT1 mRNA is NM_001382430.1 (SEQ ID NO: 13) and protein is NP_001014431.1 (SEQ ID NO: 14).

### III. PROTEIN DELIVERY

The present disclosure, in one aspect, relates to the production and formulation of transcription factors as well as their delivery to cells, tissues or subjects. In general, recombinant production of proteins is well known and is therefore no described in detail here. The discussion of nucleic acids and expression vectors, found below, is however incorporated in this discussion.

### A. Purification of Proteins

It will be desirable to purify proteins according to the present disclosure. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present disclosure concern the purification, and in particular embodiments, the substantial purification, of an encoded protein or peptide. The term “purified protein” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein composition that has been subjected to fractionation to remove various other components, and which composition substantially retains its expressed biological activity. Where the term “substantially purified” is used, this designation will refer to a composition in which the protein forms the major component of the composition, such as constituting about 50%, about 60%, about 70%, about 80%, about 90%, about 95% or more of the proteins in the composition.

Various methods for quantifying the degree of purification of the protein will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide. There is no general requirement that the protein always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present disclosure is discussed below.

### B. Cell Permeability Peptides

The present disclosure contemplates the use of a cell permeability peptide (also called a cell delivery peptide, or cell transduction domain) linked to transcription factors. Such domains have been described in the art and are generally characterized as short amphipathic or cationic peptides and peptide derivatives, often containing multiple lysine and arginine resides (Fischer, 2007). Other examples are known in the art.

### C. Protein Delivery

In general, proteins are delivered to cells as a formulation that promotes entry of the proteins into a cell of interest. In a most basic form, lipid vehicles such as liposomes. For example, liposomes, which are artificially prepared vesicles made of lipid bilayers have been used to delivery a variety of drugs. Liposomes can be composed of naturally-derived phospholipids with mixed lipid chains (like egg phosphatidylethanolamine) or other surfactants. In particular, liposomes containing cationic or neutural lipids have been used in the formulation of drugs. Liposomes should not be confused with micelles and reverse micelles composed of monolayers, which also can be used for delivery.

A wide variety of commercial formulations for protein delivery are well known including PULSin™, Lipodin-Pro, Carry-Max®, Pro-DeliverIN, PromoFectin, Pro-Ject, Chariot™ Protein Delivery reagent, BioPORTER™, and others.

Nanoparticles are generally considered to be particulate substances having a diameter of 100 nm or less. In contrast to liposomes, which are hollow, nanoparticles tend to be solid. Thus, the drug will be less entrapped and more either embedded in or coated on the nanoparticle. Nanoparticles can be made of metals including oxides, silica, polymers such as polymethyl methacrylate, and ceramics. Similarly, nanoshells are somewhat larger and encase the delivered substances with these same materials. Either nanoparticles or nanoshells permit sustained or controlled release of the peptide or mimetic, and can stabilize it to the effects of in vivo environment.

### IV. NUCLEIC ACID DELIVERY

As discussed above, in certain embodiments, expression cassettes are employed to express a transcription factor product, either for subsequent purification and delivery to a cell/subject, or for use directly in a genetic-based delivery approach. Expression requires that appropriate signals be provided in the vectors, and include various regulatory elements such as enhancers/promoters from both viral and mammalian sources that drive expression of the genes of interest in cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

### A. Regulatory Elements

Throughout this application, the term “expression cassette” is meant to include any type of genetic construct containing a nucleic acid coding for a gene product in which part or all of the nucleic acid encoding sequence is capable of being transcribed and translated, i.e., is under the control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene. An “expression vector” is meant to include expression cassettes comprised in a genetic construct that is capable of replication, and thus including one or more of origins of replication, transcription termination signals, poly-A regions, selectable markers, and multipurpose cloning sites.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, viral promotes such as the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of promoters/enhancers and inducible promoters/enhancers that could be used in combination with the nucleic acid encoding a gene of interest in an expression construct (Table 2 and Table 3). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 2
Promoter and/or Enhancer

| Promoter/Enhancer | References |
|---|---|
| Immunoglobulin Heavy Chain | Banerji et al., 1983; Gilles et al., 1983; Grosschedl |
| | et al., 1985; Atchinson et al., 1986, 1987; Imler et |
| | al., 1987; Weinberger et al., 1984; Kiledjian et al., |
| | 1988; Porton et al.; 1990 |
| Immunoglobulin Light Chain | Queen et al., 1983; Picard and Schaffner, 1984 |
| T-Cell Receptor | Luria et al., 1987; Winoto et al., 1989; Redondo et |
| | al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; |
| | Johnson et al., 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere and Tilghman et al., |
| | 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion | Hirsh et al., 1990 |
| Molecule (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth | Pech et al., 1989 |
| Factor (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr and Clarke, 1986; |
| | Imbra et al., 1986; Kadesch et al., 1986; Wang and |
| | Calame, 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen et |
| | al., 1986; Satake et al., 1988; Campbell and |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et |
| | al., 1988; Reisman et al., 1989 |
| Papilloma Virus | Campo et al., 1983; Lusky et al., 1983; Spandidos |
| | and/or Wilkie, 1983; Spalholz et al., 1985; Lusky |
| | and Botchan, 1986; Cripe et al., 1987; Gloss et al., |
| | 1987; Hirochika et al., 1987; Stephens et al., 1987 |
| Hepatitis B Virus | Bulla et al., 1986; Jameel et al., 1986; Shaul et al., |
| | 1987; Spandau and Lee, 1988; Vannice et al., 1988 |
| Human Immunodeficiency | Muesing et al., 1987; Hauber et al., 1988; |
| Virus | Jakobovits et al., 1988; Feng and Holland., 1988; |
| | Takebe et al., 1988; Rosen et al., 1988; Berkhout et |
| | al., 1989; Laspia et al., 1989; Sharp et al., 1989; |
| | Braddock et al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

TABLE 3
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart et |
| | | al., 1985; Imagawa et al., |
| | | 1987, Karin et al., 1987; |
| | | Angel et al., 1987b; |
| | | McNeall et al., 1989 |
| MMTV (mouse mammary | Glucocorticoids | Huang et al., 1981; Lee et |
| tumor virus) | | al., 1981; Majors et al., |
| | | 1983; Chandler et al., |
| | | 1983; Ponta et al., 1985; |
| | | Sakai et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | ElA, SV40 Large T Antigen | Taylor et al., 1989, 1990a, |
| | | 1990b |
| Proliferin | Phorbol Ester-TPA | Mordacq et al., 1989 |
| Tumor Necrosis Factor | PMA | Hensel et al., 1989 |
| Thyroid Stimulating | Thyroid Hormone | Chatterjee et al., 1989 |
| Hormone α Gene | | |

Of particular interest are fibroblast specific promoters, such as Fibroblast-Specific Protein 1 (FSP1) promoter (Okada et al., 1998); collagen 1A1 (COL1A1) promoter (Hitraya et al., 1998) and Periostin (Postn) promoter (Joseph et al., 2008). Other promoters include muscle specific promoters and cardiac specific promoters such as the myosin light chain-2 promoter (Franz et al., 1994; Kelly et al., 1995), the α-actin promoter (Moss et al., 1996), the troponin 1 promoter (Bhavsar et al., 1996); the Na⁺/Ca²⁺ exchanger promoter (Barnes et al., 1997), the dystrophin promoter (Kimura et al., 1997), the α7 integrin promoter (Ziober and Kramer, 1996), the brain natriuretic peptide promoter (LaPointe et al., 1996), the αB-crystallin/small heat shock protein promoter (Gopal-Srivastava, 1995), α-myosin heavy chain promoter (Yamauchi-Takihara et al., 1989) and the ANF promoter (LaPointe et al., 1988).

Where a cDNA insert is employed, one will typically desire to include a polyadenylation signal to effect proper polyadenylation of the gene transcript. The nature of the polyadenylation signal is not believed to be crucial to the successful practice of the disclosure, and any such sequence may be employed such as human growth hormone and SV40 polyadenylation signals. Also contemplated as an element of the expression cassette is a terminator. These elements can serve to enhance message levels and to minimize read through from the cassette into other sequences.

### B. Multigene Constructs and IRES

In certain embodiments of the disclosure, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

### C. Delivery of Expression Vectors

There are a number of ways in which expression vectors may introduced into cells. In certain embodiments of the disclosure, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

One of the preferred methods for in vivo delivery involves the use of an adeno-associated virus (AAV) expression vector. AAV can infect non-dividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

Another expression vector may comprise a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

There are certain limitations to the use of retrovirus vectors in all aspects of the present disclosure. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif vpr, vpu and nef are deleted making the vector biologically safe.

Recombinant lentiviral vectors are capable of infecting non-dividing cells and can be used for both in vivo and ex vivo gene transfer and expression of nucleic acid sequences. The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTR's serve to promote transcription and polyadenylation of the virion RNA's. The LTR contains all other cis-acting sequences necessary for viral replication. Lentiviruses have additional genes including vif vpr, tat, rev, vpu, nef and vpx.

Lentiviral vectors are known in the art, see Naldini et al., (1996); Zufferey et al., (1997); U.S. Pat. Nos. 6,013,516; and 5,994,136. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest.

Other viral vectors may be employed as expression constructs in the present disclosure. Vectors derived from viruses such as vaccinia virus (Ridgeway, 1988; Baichwal and Sugden, 1986; Coupar et al., 1988) and herpesviruses may be employed. They offer several attractive features for various mammalian cells (Friedmann, 1989; Ridgeway, 1988; Baichwal and Sugden, 1986; Coupar et al., 1988; Horwich et al., 1990).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present disclosure. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Further, non-viral delivery of RNA expression constructs through modified RNAs (modRNAs), messenger RNAs (mRNAs), and micro-RNAs (miRNAs) via but not limited to endosomal, liposomal, and nanoparticles are contemplated by this present disclosure (Durymanov and Reineke, 2018). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). Further, nucleic acid encoding the transcript related to the gene of interest may be delivered for gene augmentation via direct translation. In yet further embodiments, the nucleic acid may be stably maintained in the cell as a separate, episomal segment of DNA or RNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the disclosure, the expression construct may simply consist of naked recombinant DNA, RNA, or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA or RNA encoding a gene or transcript of interest may also be transferred in a similar manner in vivo and express the gene product.

In still another embodiment of the disclosure for transferring a naked DNA or modified RNA expression construct into cells may involve particle bombardment. This method depends on the ability to accelerate DNA or RNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA or RNA encoding a particular gene or transcript may be delivered via this method and still be incorporated by the present disclosure.

In a further embodiment of the disclosure, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA or lipofectamine-RNA complexes.

Liposome-mediated nucleic acid delivery and expression of foreign DNA or RNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection. A reagent known as Lipofectamine 2000™ is widely used and commercially available.

In certain embodiments of the disclosure, the liposome may be complexed with a hemagglutinating virus (HVJ). This has been shown to facilitate fusion with the cell membrane and promote cell entry of liposome-encapsulated nucleic acids (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present disclosure. Where a bacterial promoter is employed in the DNA or RNA construct, it also will be desirable to include within the liposome an appropriate bacterial polymerase.

Other expression constructs which can be employed to deliver a nucleic acid encoding a particular gene or transcript into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

### V. METHODS OF TREATING MYOCARDIAL INFARCTION

As discussed above, the present disclosure provides for new post-MI therapies. In one embodiment of the present disclosure, methods for the treatment of subjects following an MI provides for one or more of the following outcomes as compared to an untreated patient: increased exercise capacity, increased blood ejection volume, decreased left ventricular end diastolic pressure, decreased pulmonary capillary wedge pressure, increased cardiac output, improved cardiac index, decreased pulmonary artery pressures, decreased left ventricular end systolic and diastolic dimensions, and decreased left ventricular wall stress, decreased wall tension and decreased wall thickness-same for right ventricle. In addition, the treatment may prevent progression to cardiac hypertrophy, ventricular dilation, and ultimately heart failure.

Treatment regimens would vary depending on the clinical situation. However, in general, the treatment would begin at a time following an MI when the patient has been stabilized, but before significant cardiac fibroblast mobilization and scarring has begun. The patient may or may not be undergoing one or more other therapies for either prevention or treatment of an MI, or prevention or treatment of MI-related sequelae. This would mean initiating a treatment within about 24, 36, 48, 72, 96 hours of an MI, or within about 5, 6, 7, 8, 9 or 10 days of an MI. The therapy may continue for as long as cardiac fibroblasts would be active within the ischemic zone, such as up to 7 days, 14 days 21 days, 28 days, 1 month, 2 months, 3 months or longer. Further, the therapy may continue to be effective in the setting of chronic ischemic heart failure, or indefinitely following a known or presumably remote MI event.

### A. Combined Therapies

In another embodiment, it is envisioned to use the reprogramming factor therapy of the present disclosure in combination with other MI, post-MI, and ischemic heart failure therapeutic modalities, such as those discussed above. Combinations may be achieved by contacting cardiac cells/patients with a single composition or pharmacological formulation that includes both agents, or by contacting the cell with multiple distinct compositions or formulations, at the same time, wherein one composition includes the expression construct and the other includes the agent. Alternatively, the therapy using reprogramming factors may precede or follow administration of the other agent(s) by intervals ranging from minutes to weeks to years. In embodiments where the other agent and reprogramming factors are applied separately to the cardiac cells/patient, one would generally ensure that a significant period of time did not expire between the time of each delivery, such that the agent and reprogramming factors would still be able to exert an advantageously combined effect on the cell. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either reprogramming factors, or the other agent will be desired. In this regard, various combinations may be employed. By way of illustration, where the reprogramming factors are “A” and the other agent is “B,” the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are likewise contemplated. One particular combination therapy involves small molecules, micro-RNAs, and anti-inflammatory agents, such as steroids or NSAIDs. Other traditional cardiac therapies are discussed below, and may also be usefully combined with the reprogramming factors discussed above.

### B. Standard MI Therapeutic Intervention

Therapies for acute myocardial infarction are designed to restore perfusion as soon as possible to rescue the infracted myocardium. This is typically done by pharmaceutical intervention or by mechanical means, such as percutaneous coronary intervention (PCI) or coronary artery bypass grafting. Recent studies suggest that these treatments are more effective if the following guidelines are followed for ST-elevation myocardial infarction: <90 min for PCI and <30 min for lytics. Treatments outside these windows were associated with increased mortality and significantly increased risk of readmission for acute myocardial infarction or heart failure.

### 1. Drug Therapies

Thrombolytic therapy improves survival rates in patients with acute myocardial infarction if administered in a timely fashion in the appropriate group of patients. If PCI capability is not available within 90 minutes, then choice is to administer thrombolytics within 12 hours of onset of symptoms in patients with ST-segment elevation greater than 0.1 mV in 2 or more contiguous ECG leads, new left bundle-branch block (LBBB), or anterior ST depression consistent with posterior infarction. Tissue plasminogen activator (t-PA) is preferred over streptokinase as achieving a higher rate of coronary artery patency; however, the key lies in speed of the delivery.

Aspirin has been shown to decrease mortality and re-infarction rates after myocardial infarction. Again, delivery should be immediate, which should be chewed if possible. The treatment should continues indefinitely in the absence of obvious contraindication, such as a bleeding tendency or an allergy. Clopidogrel may be used as an alternative in cases of a resistance or allergy to aspirin (dose of 300 mg), but a higher dose of clopidogrel may have added benefit.

Platelet glycoprotein (GP) IIb/IIIa-receptor antagonist is another therapy in patients with continuing ischemia or with other high-risk features and to patients in whom a percutaneous coronary intervention (PCI) is planned. Eptifibatide and tirofiban are approved for this use, and abciximab also can be used for 12-24 hours in patients with unstable angina or NSTEMI in whom a PCI is planned within the next 24 hours.

Heparin and other anticoagulant agents have an established role as adjunct agents in patients receiving t-PA, but not in patients receiving streptokinase. Heparin is also indicated in patients undergoing primary angioplasty. Low molecular-weight heparins (LMWHs) have been shown to be superior to UFHs in patients with unstable angina or NSTEMI. Bivalirudin, a direct thrombin inhibitor, has shown promise in STEMI if combined with high-dose clopidogrel.

Nitrates have no apparent impact on mortality rate in patients with ischemic syndromes, but they are useful in symptomatic relief and preload reduction, so much so that all patients with acute myocardial infarction are given nitrates within the first 48 hours of presentation, unless contraindicated (i.e., in RV infarction). Beta-blockers may reduce the rates of reinfarction and recurrent ischemia, and thus are administered to patients with MIs unless a contraindication is present.

Beta blockers reduce mortality rates after myocardial infarction and thus are administered as soon as possible as long as there are no contraindications present such as acute heart failure are and the patient remains stable, and are to be continued for at least two years after myocardial infarction.

### 2. PCI and Other Surgical Intervention

PCI is the treatment of choice in most patients with STEMI, assuming a door to balloon time of less than 90 minutes. PCI provides greater coronary patency (>96% thrombolysis), lower risk of bleeding, and instant knowledge about the extent of the underlying disease. Studies have shown that primary PCI has a mortality benefit over thrombolytic therapy. The choice of primary PCI should be individualized to each patient's presentation and timing. Primary PCI is also the treatment of choice in patients with cardiogenic shock, patients in whom thrombolysis failed, and those with high risk of bleeding or contraindications to thrombolytic therapy.

Emergent or urgent coronary artery graft bypass surgery is indicated in patients who have left main or three vessel disease in conjunction with diabetes, in whom angioplasty fails, and in patients who develop mechanical complications such as a VSD, LV, or papillary muscle rupture.

### C. Pharmacological Therapeutic Agents

Pharmacological therapeutic agents and methods of administration, dosages, etc., are well known to those of skill in the art (see for example, the “Physicians Desk Reference”, Klaassen's “The Pharmacological Basis of Therapeutics”, “Remington's Pharmaceutical Sciences”, and “The Merck Index, Eleventh Edition”, incorporated herein by reference in relevant parts), and may be combined with the disclosure in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art. It should be noted that the below therapeutic agents possess varying levels of evidence for efficacy in management of acute myocardial infarction or heart failure.

In addition to the reprogramming factors of the present disclosure, it should be noted that any of the following may be used to develop new therapeutic regimens in combination with the transcription factors.

### 1. Antihyperlipoproteinemics

In certain embodiments, administration of an agent that lowers the concentration of one of more blood lipids and/or lipoproteins, known herein as an “antihyperlipoproteinemic,” may be combined with a cardiovascular therapy according to the present disclosure, particularly in treatment of athersclerosis and thickenings or blockages of vascular tissues. In certain aspects, an antihyperlipoproteinemic agent may comprise an aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof.

a. Aryloxyalkanoic Acid/Fibric Acid Derivatives

Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate.

b. Resins/Bile Acid Sequesterants

Non-limiting examples of resins/bile acid sequesterants include cholestyramine (cholybar, questran), colestipol (colestid) and polidexide.

c. HMG CoA Reductase Inhibitors

Non-limiting examples of HMG CoA reductase inhibitors include lovastatin (mevacor), pravastatin (pravochol) or simvastatin (zocor).

d. Nicotinic Acid Derivatives

Non-limiting examples of nicotinic acid derivatives include nicotinate, acepimox, niceritrol, nicoclonate, nicomol and oxiniacic acid.

e. Thryroid Hormones and Analogs

Non-limiting examples of thyroid hormones and analogs thereof include etoroxate, thyropropic acid and thyroxine.

f. Miscellaneous Antihyperlipoproteinemics

Non-limiting examples of miscellaneous antihyperlipoproteinemics include acifran, azacosterol, benfluorex, β-benzalbutyramide, carnitine, chondroitin sulfate, clomestrone, detaxtran, dextran sulfate sodium, 5,8, 11, 14, 17-eicosapentaenoic acid, eritadenine, furazabol, meglutol, melinamide, mytatrienediol, ornithine, γ-oryzanol, pantethine, pentaerythritol tetraacetate, α-phenylbutyramide, pirozadil, probucol (lorelco), β-sitosterol, sultosilic acid-piperazine salt, tiadenol, triparanol and xenbucin.

### 2. Antiarteriosclerotics

Non-limiting examples of an antiarteriosclerotic include pyridinol carbamate.

### 3. Antithrombotic/Fibrinolytic Agents

In certain embodiments, administration of an agent that aids in the removal or prevention of blood clots may be combined with administration of a modulator, particularly in treatment of athersclerosis and vasculature (e.g., arterial) blockages. Non-limiting examples of antithrombotic and/or fibrinolytic agents include anticoagulants, anticoagulant antagonists, antiplatelet agents, thrombolytic agents, thrombolytic agent antagonists or combinations thereof.

In certain aspects, antithrombotic agents that can be administered orally, such as, for example, aspirin and wafarin (coumadin), are preferred.

a. Anticoagulants

A non-limiting example of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol, warfarin, and direct oral anticoagulants such as rivaroxaban and apixaban.

b. Antiplatelet Agents

Non-limiting examples of antiplatelet agents include aspirin, dipyridamole (persantin), heparin, clopidogrel, prasugrel, and ticagrelor.

c. Thrombolytic Agents

Non-limiting examples of thrombolytic agents include tissue plaminogen activator (activase), plasmin, pro-urokinase, urokinase (abbokinase) streptokinase (streptase), anistreplase/APSAC (eminase).

### 4. Blood Coagulants

In certain embodiments wherein a patient is suffering from a hemorrhage or an increased likelihood of hemorrhaging, an agent that may enhance blood coagulation may be used. Non-limiting examples of a blood coagulation promoting agent include thrombolytic agent antagonists and anticoagulant antagonists.

a. Anticoagulant Antagonists

Non-limiting examples of anticoagulant antagonists include protamine and vitamin K1.

b. Thrombolytic Agent Antagonists and Antithrombotics

Non-limiting examples of thrombolytic agent antagonists include amiocaproic acid (amicar) and tranexamic acid (amstat). Non-limiting examples of antithrombotics include anagrelide, argatroban, cilstazol, daltroban, defibrotide, enoxaparin, fraxiparine, indobufen, lamoparan, ozagrel, picotamide, plafibride, tedelparin, ticlopidine and triflusal.

### 5. Antiarrhythmic Agents

Non-limiting examples of antiarrhythmic agents include Class I antiarrythmic agents (sodium channel blockers), Class II antiarrythmic agents (beta-adrenergic blockers), Class II antiarrythmic agents (repolarization prolonging drugs), Class IV antiarrhythmic agents (calcium channel blockers) and miscellaneous antiarrythmic agents.

a. Sodium Channel Blockers

Non-limiting examples of sodium channel blockers include Class IA, Class IB and Class IC antiarrhythmic agents. Non-limiting examples of Class IA antiarrhythmic agents include disppyramide (norpace), procainamide (pronestyl) and quinidine (quinidex). Non-limiting examples of Class IB antiarrhythmic agents include lidocaine (xylocaine), tocainide (tonocard) and mexiletine (mexitil). Non-limiting examples of Class IC antiarrhythmic agents include encainide (enkaid) and flecainide (tambocor).

b. Beta Blockers

Non-limiting examples of a beta blocker, otherwise known as a β-adrenergic blocker, a β-adrenergic antagonist or a Class II antiarrhythmic agent, include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.

c. Repolarization Prolonging Agents

Non-limiting examples of an agent that prolong repolarization, also known as a Class III antiarrhythmic agent, include amiodarone (cordarone) and sotalol (betapace).

d. Calcium Channel Blockers/Antagonist

Non-limiting examples of a calcium channel blocker, otherwise known as a Class IV antiarrythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexiline. In certain embodiments a calcium channel blocker comprises a long-acting dihydropyridine (nifedipine-type) calcium antagonist.

e. Miscellaneous Antiarrhythmic Agents

Non-limiting examples of miscellaneous antiarrhymic agents include adenosine (adenocard), digoxin (lanoxin), acecainide, ajmaline, amoproxan, aprindine, bretylium tosylate, bunaftine, butobendine, capobenic acid, cifenline, disopyranide, hydroquinidine, indecainide, ipatropium bromide, lidocaine, lorajmine, lorcainide, meobentine, moricizine, pirmenol, prajmaline, propafenone, pyrinoline, quinidine polygalacturonate, quinidine sulfate and viquidil.

### 6. Antihypertensive Agents

Non-limiting examples of antihypertensive agents include sympatholytic, alpha/beta blockers, alpha blockers, anti-angiotensin II agents, beta blockers, calcium channel blockers, vasodilators and miscellaneous antihypertensives.

a. Alpha Blockers

Non-limiting examples of an alpha blocker, also known as an α-adrenergic blocker or an α-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

b. Alpha/Beta Blockers

In certain embodiments, an antihypertensive agent is both an alpha and beta adrenergic antagonist. Non-limiting examples of an alpha/beta blocker comprise labetalol (normodyne, trandate).

c. Anti-Angiotension II Agents

Non-limiting examples of anti-angiotension II agents include angiotensin converting enzyme inhibitors and angiotension II receptor antagonists. Non-limiting examples of angiotension converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBs), include angiocandesartan, eprosartan, irbesartan, losartan and valsartan. Non-limiting examples of combination neprilysin inhibitors and ARBs include sacubitril/valsartan.

d. Sympatholytics

Non-limiting examples of a sympatholytic include a centrally acting sympatholytic or a peripherally acting sympatholytic. Non-limiting examples of a centrally acting sympatholytic, also known as an central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a β-adrenergic blocking agent or a alpha1-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alpha1-adrenergic blocker include prazosin (minipress), doxazocin (cardura) and terazosin (hytrin).

e. Vasodilators

In certain embodiments a cardiovasculator therapeutic agent may comprise a vasodilator (e.g., a cerebral vasodilator, a coronary vasodilator or a peripheral vasodilator). In certain preferred embodiments, a vasodilator comprises a coronary vasodilator. Non-limiting examples of a coronary vasodilator include amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(β-diethylaminoethyl ether), hexobendine, itramin tosylate, khellin, lidoflanine, mannitol hexanitrane, medibazine, nicorglycerin, pentaerythritol tetranitrate, pentrinitrol, perhexiline, pimefylline, trapidil, tricromyl, trimetazidine, trolnitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

f. Miscellaneous Antihypertensives

Non-limiting examples of miscellaneous antihypertensives include ajmaline, γ-aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quanternary ammonium compound, a reserpine derivative or a suflonamide derivative.

Arylethanolamine Derivatives. Non-limiting examples of arylethanolamine derivatives include amosulalol, bufuralol, dilevalol, labetalol, pronethalol, sotalol and sulfinalol.

Benzothiadiazine Derivatives. Non-limiting examples of benzothiadiazine derivatives include althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide.

N-carboxyalkyl(peptide/lactam) Derivatives. Non-limiting examples of N-carboxyalkyl(peptide/lactam) derivatives include alacepril, captopril, cilazapril, delapril, enalapril, enalaprilat, fosinopril, lisinopril, moveltipril, perindopril, quinapril and ramipril.

Dihydropyridine Derivatives. Non-limiting examples of dihydropyridine derivatives include amlodipine, felodipine, isradipine, nicardipine, nifedipine, nilvadipine, nisoldipine and nitrendipine.

Guanidine Derivatives. Non-limiting examples of guanidine derivatives include bethanidine, debrisoquin, guanabenz, guanacline, guanadrel, guanazodine, guanethidine, guanfacine, guanochlor, guanoxabenz and guanoxan.

Hydrazines/Phthalazines. Non-limiting examples of hydrazines/phthalazines include budralazine, cadralazine, dihydralazine, endralazine, hydracarbazine, hydralazine, pheniprazine, pildralazine and todralazine.

Imidazole Derivatives. Non-limiting examples of imidazole derivatives include clonidine, lofexidine, phentolamine, tiamenidine and tolonidine.

Quanternary Ammonium Compounds. Non-limiting examples of quanternary ammonium compounds include azamethonium bromide, chlorisondamine chloride, hexamethonium, pentacynium bis(methylsulfate), pentamethonium bromide, pentolinium tartrate, phenactropinium chloride and trimethidinium methosulfate.

Reserpine Derivatives. Non-limiting examples of reserpine derivatives include bietaserpine, deserpidine, rescinnamine, reserpine and syrosingopine.

Suflonamide Derivatives. Non-limiting examples of sulfonamide derivatives include ambuside, clopamide, furosemide, indapamide, quinethazone, tripamide and xipamide.

g. Vasopressors

Vasopressors generally are used to increase blood pressure during shock, which may occur during a surgical procedure. Non-limiting examples of a vasopressor, also known as an antihypotensive, include amezinium methyl sulfate, angiotensin amide, dimetofrine, dopamine, etifelmin, etilefrin, gepefrine, metaraminol, midodrine, norepinephrine, pholedrine and synephrine.

### 7. Treatment Agents for Congestive Heart Failure

Non-limiting examples of agents for the treatment of congestive heart failure include anti-angiotension II agents, afterload-preload reduction treatment, diuretics and inotropic agents.

a. Afterload-Preload Reduction

In certain embodiments, an animal patient that can not tolerate an angiotension antagonist may be treated with a combination therapy. Such therapy may combine administration of hydralazine (apresoline), isosorbide dinitrate (isordil, sorbitrate), ACE inhibitors (lisinopril, enalapril, captopril), ARBs (losartan), or neprilysin inhibitor/ARB combination therapies (ARNi) (sacubitril-valsartan).

b. Diuretics

Non-limiting examples of a diuretic include a thiazide or benzothiadiazine derivative (e.g., althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furterene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzolimine, perhexiline, ticrnafen and urea.

c. Inotropic Agents

Non-limiting examples of a positive inotropic agent, also known as a cardiotonic, include acefylline, an acetyldigitoxin, 2-amino-4-picoline, amrinone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include amrinone (inocor).

e. SGLT2 Inhibitors

SGLT2 inhibitors have been shown to decrease mortality in patients with heart failure and coronary artery disease. Non-limiting examples of SGLT2 inhibitors include empagliflozin, dapagliflozin, and canagliflozin.

f. GLP-1 Agonists

GLP-1 agonists are commonly used in patients with coronary artery disease or prior MI with or without heart failure. Non-limiting examples of GLP-1 agonists include liraglutide and semaglutide.

g. Antianginal Agents

Antianginal agents may comprise organonitrates, calcium channel blockers, beta blockers and combinations thereof.

Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).

### D. Surgical Therapeutic Agents

In certain aspects, the secondary therapeutic agent may comprise a surgery of some type, such as PCI. Surgery, and in particular a curative surgery, may be used in conjunction with other therapies, such as the present disclosure and one or more other pharmacologic agents. Such surgical approaches for vascular and cardiovascular diseases and disorders are well known to those of skill in the art, and are described elsewhere in this document.

### E. Drug Formulations and Routes for Administration to Patients

Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render drugs, proteins or delivery vectors stable and allow for uptake by target cells. Aqueous compositions of the present disclosure comprise an effective amount of the drug, vector or proteins, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present disclosure, its use in therapeutic compositions is contemplated. Supplementary active ingredients also can be incorporated into the compositions, provided they do not inactivate the vectors or cells of the compositions.

The active compositions of the present disclosure may include classic pharmaceutical preparations. Administration of these compositions according to the present disclosure may be via any common route so long as the target tissue is available via that route. This includes oral, nasal, or buccal. Alternatively, administration may be by intradermal, subcutaneous, intramuscular, intraperitoneal or intravenous injection, or by direct injection into cardiac tissue. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present disclosure generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### VI. EXAMPLES

The following examples are included to further illustrate various aspects of the disclosure. It should be appreciated by those of skill in the art that the techniques disclosed in the examples which follow represent techniques and/or compositions discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Methods

Mice. All experiments involving animals were approved by the Institutional Animal Care and Use Committee at the University of Texas Southwestern Medical Center. All mice used in this study were housed at the Animal Resource Center at the University of Texas Southwestern Medical Center and bred inside a SPF facility with 12 h light/dark cycles and monitored daily with no health problems reported. All animals were housed in groups of maximum five per cage with ad libitum access to food and water. The temperature and humidity of all animal rooms is electronically monitored and regulated. Personal protective equipment (PPE) is required in the animal facility and provided at the entrance of the animal facility. α-MHC-GFP mice were maintained on a C57BL/6 background (Song et al., 2012).

Isolation and culture of mouse fibroblasts. Adult mouse tail tip fibroblasts and cardiac fibroblasts from 4-6 weeks old male and female C57BL/6 or α-MHC-GFP mice were prepared as previously described and cultured in fibroblast growth medium until experiments were performed (Wamstad et al., 2012). MEFs derived from embryos of C57BL/6 or α-MHC-GFP timed pregnant female were harvested at E13.5-14.5 and were prepared as previously described (Nam et al., 2014). All primary cells were maintained in DMEM with 10% FBS and 1% penicillin/streptomycin.

Retrovirus production and cardiac reprogramming. Generation of retroviral expression constructs encoding Gata4, Hand2, Mef2c, Tbx5, Akt1, and PHF7 has been previously described (Song et al., 2012; Zhou et al., 2015; Wamstad et al., 2012). Retroviral constructs of shRNA targeting Smarcd3 and Scramble sequence 5′-CTACACAAATCAGCGATTTcgaaAAATCGCTGATTTGTGTAG-3′ and sequence 5′-GGTGGAATTCAGTGGTCAAGAcgaaTCTTGACCACTGAATTCCACC-3′ were cloned into an entry vector using BLOCK-iT U6 Entry Vector Kit (Thermo Scientific) and recombined into pMXs-GW vector by Gateway cloning. pMXs-GW was a gift from Dr. Shinya Yamanaka (Addgene plasmid #18656) (Takahashi & Yamanaka, 2006). Retroviruses were produced by using Platinum E cells, as previously described (Wamstad et al., 2012). Briefly, retroviral constructs were transfected into Platinum E cells using FuGENE 6 transfection reagent. Twenty-four hours after transfection, wild-type or α-MHC-GFP fibroblasts were seeded into culture dishes or plates that were precoated with SureCoat (Cellutron) or Matrigel (Corning). Forty-eight hours after transfection, the viral medium was filtered through a 0.45-μm filter and polybrene was added at a concentration of 8 μg/mL. Then fibroblasts were infected by replacing growth medium with the above viral mixture. The viral infection was serially repeated twice and twenty-four hours after the second infection, the viral medium was replaced with induction medium composed of DMEM/199 (4:1), 10% FBS, 5% horse serum, 1% penicillin/streptomycin, 1% nonessential amino acids, 1% essential amino acids, 1% B-27, 1% insulin-selenium-transferrin, 1% vitamin mixture, and 1% sodium pyruvate (Invitrogen). Induction medium was replaced every two to three days until experiments were performed. For human cardiac fibroblast reprogramming, one day after viral transduction (day 1), the virus medium was replaced by induction medium composed of DMEM/199 (4:1), 10% FBS, 1% nonessential amino acids, 1% penicillin/streptomycin, for every two days until day 4. On day 4, the medium was changed to 75% induction media and 25% RPMI+B27. On day 7, the medium was changed to 50% iCM and 50% RPMI+B27. On day 11, the medium was changed to 25% iCM and 75% RPMI+B27. On day 14, medium was changed to RPMI+B27+FFV (10 ng/ml rhFGF, 15 ng/ml rhFGF-10, and 5 ng/ml rhVEGF) every other day until day 21. Immunostaining and gene expression analysis were performed using day 21 samples.

Quantitative mRNA measurement. Total RNA was extracted using TRIzol (Invitrogen) according to the vender's protocol. RNAs were reverse-transcribed to cDNA using iScript Supermix (Bio-Rad). qPCR was performed using KAPA SYBR Fast (Kapa Biosystems) and gene expression was analyzed by the Ct method. Realtime qPCR was performed using Taq-man probes (Applied Biosystems) for Gata4 targeted at the 3′ UTR. SYBR primers were used for Myh6 Fw 5′-GCC CAG TAC CTC CGA AAG TC-3′ and Rv 5′-GCC TTA ACA TAC TCC TTG TC-3′, Tnnt2 Fw 5′-GTA GAG GAC ACC AAA CCC AAG-3′ and Rv 5′-GAG TCT GTA GCT CAT TCA GGT C-3′, Actc1 Fw 5′-CGG ACA ATT TCA CGT TCA GCA-3′ and Rv 5′-CTG GAT TCT GGC GAT GGT GTA-3′, Tnni3 Fw 5′-TCT GCC AAC TAC CGA GCC TAT-3′ and Rv-5′CTC TTC TGC CTC TCG TTC CAT-3′, and Nppb Fw 5′-GAGGTCACTCCTATCCTCTGG-3′ and Rv 5′-GCCATTTCCTCCGACTTTTCTC-3′, PHF7 Fw 5′-GACTAGGAGGGTAACCCAGAG-3′ and Rv 5′-TGCACGCTGATATTGTCTTTCT-3′, Smarcd3 Fw 5′-CCC GAG TCC CAG GCT TAC A-3′ and Rv 5′-GCT TTC GCT TTT GCT TCA TGG-3′. For input normalization, the inventors used 18S (Applied Biosystems, 4319413E) or Gapdh Fw 5′-AGG TCG GTG TGA ACG GAT TTG-3′ and Rv 5′-TGT AGA CCA TGT AGT TGA GGT CA-3′.

Immunocytochemistry. Immunocytochemistry was performed as previously described (Zhou et al., 2015). Briefly, cells were fixed in 4% PFA for 15 min at room temperature and blocked with 5% goat serum. Fixed cells were then incubated on a rotator with mouse monoclonal anti-Tnnt2 antibody (1:500, Thermo Scientific, MA5-12960), rabbit anti-GFP antibody (1:500, Thermo Scientific, A-11122), or mouse anti-α-actinin (1:500, Sigma, A7811) in 5% goat serum at 4° C. overnight. After three washes with PBS, cells were incubated with appropriate Alexa fluorogenic secondary antibodies (1:500, Invitrogen) at room temperature for 1 hr. Image acquisition and analysis was done on a BZ-X710 or BZ-X800 (Keyence). For quantification, cells were manually quantified and averaged to yield an individual replicate in ten randomly selected low-power fields of view from each well in three independent experiments.

Western blot analysis. HEK293 cells were transfected with plasmids using FuGene6 and cell lysates were analyzed after 48 hours of transfection. Western blot analyses were performed as previously described (Song et al., 2012). Briefly, cell lysates were prepared using RIPA buffer with complete protease inhibitor cocktail tablets (Roche). Lysates were boiled with 4× Laemmli buffer for 5 min at 95° C. Antibodies used were anti-Mef2c antibody (1:1000, Cell Signaling, 5030), anti-Gata4 antibody (1:500, Santa Cruz, sc-25310), anti-Tnnt2 antibody (1:500, Thermo Scientific, MA5-12960), anti-GFP antibody (1:500, Thermo Scientific, A-11122), anti-PHF7 antibody (1:500, LSBio, B11090), anti-Ty1 antibody (1:1000, Diagenode, C15200054), anti-myc (1:1000, Invitrogen, 46-0603), anti-FLAG (1:1000, Sigma, F7425), anti-mCherry antibody (1:1000, Abcam, ab167453), and anti-Gapdh antibody (1:1000, Merck Millipore, MAB374).

Flow cytometry. Flow cytometry was performed as previously described⁸. Briefly, cells were trypsinized, harvested, and suspended into single cells. Then cells were fixed and permeabilized using BD Cytofix/Cytoperm (BD Biosciences). Antibodies used were mouse monoclonal anti-Tnnt2 antibody (1:200, Thermo Scientific, MA5-12960), rabbit anti-GFP antibody (1:200, Thermo Scientific, A-11122), donkey anti-mouse Alexa fluor 647 (1:200, Invitrogen, A-31571) and goat anti-rabbit Alexa fluor 488 (1:200, Invitrogen, A-11008). Cells were analyzed using FACSCalibur (BD Biosciences) and FlowJo software (FLOWJO, LCC).

Beating cell analysis and calcium assay. Beating cell analyses were performed as previously described on Matrigel coated dish (Corning, 354248) (Zhou et al., 2015). Beating cells were manually counted in eight randomly selected high-power fields per well in at least three independent experiments. Calcium assay was performed as previously described on Matrigel coated dishes with some modification (Zhou et al., 2015). Fluo-4 NW Calcium Assay Kit (Thermo Scientific, F36206) was used according to the manufacturer's protocol and Ca²⁺ flux was measured on fibroblasts 10 days after retroviral treatment. Briefly, after replacing culture medium with the dye loading solution, plates were incubated at 37° C. for 30 minutes, then at room temperature for an additional 30 minutes before measurement. Ca²⁺ flux positive cells were manually counted in ten randomly selected high-power fields per well in three independent experiments.

Inducible Reprogramming Assays. PHF7 expression was temporally induced and studied in reprogramming using the Retro-X Tet-One Inducible Expression System (Takara, #634304). Inducible PHF7 expression cassette was generated by cloning pRetro-X-PHF7 using the manufacturer protocol. pRetroX-PHF7 was then retrovirally delivered to TTFs and MEFs with reprogramming factors. Doxycycline was added to doxycycline treatment groups at 1 g/mL following infection and to reprogramming induction media. Media with doxycycline was changed every two days. Control groups included no doxycycline (no dox) and sustained doxycycline (D1 on) exposure, while treatment groups included removal of doxycycline at days 3 and 10 (D3 off and D10 off, respectively) following the addition of reprogramming induction media. Gene and protein expression, calcium flux, and beating were assessed either throughout or at day 28 post-induction media, as indicated.

EdU Labeling. At day 1 post-induction media adult TTF iCLMs were treated with 10 μM EdU (Lumiprobe, 10540) for 4 hours. After that, cells were fixed with 4% PFA at room temperature for 10 min, permeabilized with 0.3% Triton X-100 in PBS, followed by EdU staining by click chemistry (a label mix containing 8 μM sulfo-Cy5-Azide, 2 mM CuSO₄·5H2O and 20 mg/mL ascorbic acid in PBS was applied to cells for 30 min). Cells were washed and nuclei were counter-stained with Hoechst (1:2000). Ten fields at 10× magnification were captured per each well. Quantification was carried out by counting the % EdU+ cells of total cells on immunofluorescent staining images.

RNA-seq sample preparation. For RNA-seq sample preparation, total RNA was extracted from TTFs seven days after retroviral transduction, using TRIzol (Invitrogen) according to the vender's protocol. Illumina RNA-seq was performed by the University of Texas Southwestern Microarray Core Facility

ChIP-seq sample preparation. For ChIP-seq sample preparation, MEFs or TTFs two days after retroviral transduction were crosslinked with 1% formaldehyde in PBS for 15 min and neutralized by the addition of glycine to a final concentration of 0.125M for 5 min. TTFs or MEFs were then harvested and washed with cold PBS for ChIP. ChIP was then performed using ChIP-IT Express kits (Active Motif) following the vender's protocol. In brief, cell lysates were sonicated (ten cycles of 30 sec on/off) to shear DNA by using Bioruptor Pico sonicator (Diagenode, B01060010). Then, chromatin was incubated with indicated antibodies overnight at 4° C. Pre-washed agarose beads (protein G) were then added to the antibody-treated chromatin, and immunoprecipitation was performed on a rotator for 3 hours at 4° C. The following antibodies were used for ChIP experiments: anti-Gata4 antibody (Santa Cruz Biotechnology, sc-1237), anti-Ty1 antibody (Diagenode, C15200054), and anti-H3K27ac antibody (Diagenode, C15410196). Chromatin was washed, eluted, and reverse-crosslinked. ChIP-seq libraries were generated using KAPA Hyper Prep Kit following the manufacturer's protocol (Kapa Biosystems), and single-end sequenced on the Illumina NextSeq500 system using the 75 bp high output sequencing kit. Subsequent massive parallel sequencing was performed at the University of Texas Southwestern Next Generation Sequencing Core Facility. For ChIP-qPCR, chromatin fragments were then analyzed by qPCR using SYBR Green fluorescence using the following primer sequences that had been previously validated: Gata4 TSS: Fw 5′-CTG GGT AGG GGC TGG AGT AG-3′, Rev 5′-CTG GCC GAG AGC AGT ACG-3′, Myh6 promoter Fw 5′-GCA GAT AGC CAG GGT TGA AA-3′ Rev 5′-TGG GTA AGG GTC ACC TCC TC-3′, Tbx5 Fw 5′-GCG AAG GGA TGT TTC AGC AC, Rev CAC GCC GTG AGT GTA GAG AA-3′.

ATAC-Seq sample preparation. ATAC-seq was performed as per Omni-Seq protocol by Corces et al (Corces et al., 2017). All ATAC-seq experiments were performed using 50,000 cells. Multiplexed paired-end 75 bp sequencing was performed using Illumina HiSeq 2500 using Nextera-compatible amplification primers.

Immunoprecipitation assays. PHF7^{FLAG-HA} was co-expressed with GFP, Gata4^{myc}, Hand2^{myc}, or Mef2c^{myc}, or SMARCD3^{FLAG-HA} was co-expressed with PHF^{3xTy1} or GFP in HEK293 cells for 72 h. Experiments involving crosslinking were adapted from Zlatic et al where cells were treated with 200 μM DSP or DMSO for 4 hours at 4 C and quenched with 25 mM Tris for 15 min (Zlatic et al., 2010). Pre-cleared lysates were incubated with FLAG magnetic beads (Sigma, M8823) overnight. The FLAG epitope was eluted using 0.5 mg/ml free 3× FLAG peptide (Sigma). The final elution and input obtained before immunoprecipitation were analyzed by Western blot using anti-myc (Invitrogen), anti-Ty1 (Diagenode), or rabbit anti-flag antibody (Sigma).

In vitro transgenic reporter assays. Putative enhancers were cloned into an hsp68-mCherry expression vector. Expression cassettes were cloned into pMXs retroviral vectors via Gateway cloning. pMXs-enhancer-hsp68-mCherry constructs were then retrovirally delivered to MEFs together with reprogramming factors and mCherry expression was investigated in iCLMs. Genomic coordinates of all enhancers are listed in Extended Data Table 3.

miniTurbo BioID assay. Proximity biotinylation (BioID) was adapted from Branon et al (Branon et al., 2018). Briefly, MEFs were infected with empty vector, pMXs-puro-PHF7-miniTurbo or AGHMT+pMXs-puro-PHF7-miniTurbo and exposed to reprogramming induction media as described above for 7 days. Cells were then exposed to 200 μM Biotin for 4 h or no biotin control. Cell lysates were extracted in 1 ml of lysis buffer (6M urea, 10% SDS, supplemented with protease inhibitor) and lysed mechanically. Lysates were added to streptavidin magnetic beads and rotated for 24 h at 4° C. (Thermo Fisher, 88816), with final elution by boiling at 95° C. for 5 min. Pulldown was assessed by silver staining (Thermo Fisher, LC6070) and peptide identification was performed by the Proteomics Core Facility at University of Texas Southwestern Medical Center. Data were normalized to total protein submitted per sample as well as empty vector/biotin negative control. Proteomics hits were validated by Western blot of submitted sample.

Bioinformatic and computational analysis. RNA-Seq analysis. RNA-seq and transcriptome analysis were performed as described in Zhou et al. Briefly, reads were aligned to the mouse reference genome GRCm38 (mm10) using the Hisat (version 2.0.0) aligner using default settings. Aligned reads were counted using featureCounts (version 1.4.6) per gene ID. Differential gene expression analysis was done using the R package edgeR (version 3.8.6). Cutoff values of fold change >2 and FDR <0.01 were used to select for differentially expressed genes. DAVID gene functional annotation and classification tool was used to annotate a list of differentially expressed genes. GO analysis was performed to determine biological functional categories and enrichment plots were performed using GSEA software.

ChIP-Seq analysis. Raw sequencing reads with >30% nucleotide with phred quality scores <20 were filtered. Single-end sequencing reads were then aligned to the mouse reference genome GRCm38 (mm10) using bowtie2 aligner (v 2.3.4.3) with default parameters. For transcription factor ChIP-seq data, peaks were called using HOMER software package (version 4.9) findpeaks command, with parameter ‘-style factor’, peaks were called with >2 fold enrichment over input controls and >4 fold enrichment over local tag count and FDR threshold was set to 10⁻³. For histone marker ChIP-Seq data, peaks were called by findpeaks command with parameter ‘-style histone’, peaks were called with >2 fold enrichment over input controls and >4 fold enrichment over local tag counts and FDR threshold set to 10⁻³. ChIP-seq peaks within a 1000 bp range were stitched together to form broad regions. To identify differential peaks between two samples, called peaks were merged from each sample and raw read count within each peak region were calculated. Then differential peaks were identified using R package DEseq version 3.8. PHF7 peaks with >2 fold change were designated as DE peaks. To identify potential TF motifs enriched in interested peaks regions, the inventors used findMotifsGenome.pl command from HOMER software package, using peak region .bed file as input, with parameter ‘-size 200’ to search for motif enrichment in 200 bp window surrounding peak center. To analyze the functional significance of bound peaks, Genomic Regions Enrichment of Annotations Tool (GREAT) was used with mm10 as the background genome and other parameters set as default. To annotate super-enhancers, ranking of super-enhancer (ROSE) algorithm was used (younglab.wi.mit.edu/super_enhancer_code.html) with H3K27ac ChIP-seq data from P4 mouse ventricle as input. Custom R script was used to plot ranking of SEs and selected SE-associated genes.

ATAC-Seq analysis. Paired-end raw reads were trimmed 30 bps from 3′end to remove possible adaptor sequences and mapped to the mouse reference genome (GRCh38/mm10) using bowtie2 (version 2.3.4.3) with parameter ‘-very-sensitive’ enabled. Read duplication and reads that mapped to chrM were removed from downstream analysis. ATAC peaks were then called using the ENCODE ATAC-seq pipeline (github.com/ENCODE-DCC/atac-seq-pipeline) with IDR threshold 0.05. Called peaks were merged from all samples and read counts were calculated and produce a raw count matrix. Differential peaks were identified using R package DEseq version 3.8. Peaks with >2-fold change were designated as DE peaks. To analyze the functional significance of peaks, Genomic Regions Enrichment of Annotations Tool (GREAT) was used with mm10 as the background genome and other parameters set as default.

Statistical analyses. All data are presented as mean±SD (error bars). For every reprogramming experiment, three different plates per group were reprogrammed in parallel, unless noted otherwise. Representative reprogramming experiments are shown, unless otherwise specified. Statistical analyses were performed using GraphPad Prism 9. P-values were calculated using Student's t-test or one-way ANOVA. Significance is depicted by *p<0.05, **p<0.01, and ***p<0.001, ****p<0.0001.

Data availability. All RNA-Seq, ChIP-Seq, ATAC-Seq data that support the findings of this study have been deposited in the Gene Expression Omnibus (GEO) under accession code GSE151328. Previously published ChIP-Seq and single cell RNA-Seq data that were re-analysed here are available under accession code GSE90893, GSE112315, GSE100471, and singlecell.stemcells.cam.ac.uk/mesp1. Source data are provided with this study. All other data supporting the findings of this study are available from the corresponding author on reasonable request.

### Example 2—Results

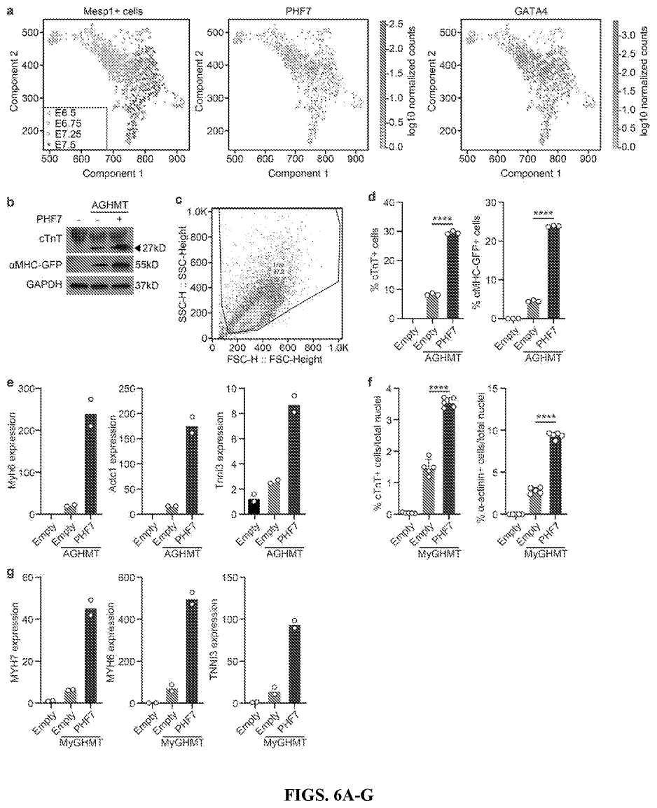

PHF7 promotes direct cardiac reprogramming. Adult fibroblast reprogramming with GMT, GHMT, or AGHMT converts only 0.5-3% of adult tail-tip fibroblasts (TTFs) to iCLMs (Ieda et al., 2010; Song et al., 2012; Zhou et al., 2016; Zhou et al., 2017). To identify potential modifiers of the barriers to reprogramming, the inventors conducted a screen of over 1000 retroviruses encoding transcription factors and epigenetic regulators in AGHMT reprogramming of adult TTFs (Zhou et al., 2017). From this screen, they identified the histone reader PHF7 as the most significant activator of direct cardiac reprogramming. A critical regulator of germline cell fate specification, PHF7 binds directly to histone H3, as well as H3K4me3 and H3K4me2 marks (Yang et al., 2012; Yang et al., 2017; Wang et al., 2019). However, its effect on chromatin structure, associated complexes, and role in cardiac biology remain undefined. Through interrogation of single cell Mesp1+ cardiac progenitor analyses from E6.5-E7.5 murine tissues, the inventors observed robust and sustained expression of Phf7 in early cardiac progenitors, suggesting that Phf7 plays an endogenous role in cardiac stem cell biology (FIG. 6A) (Lescroart et al., 2018).

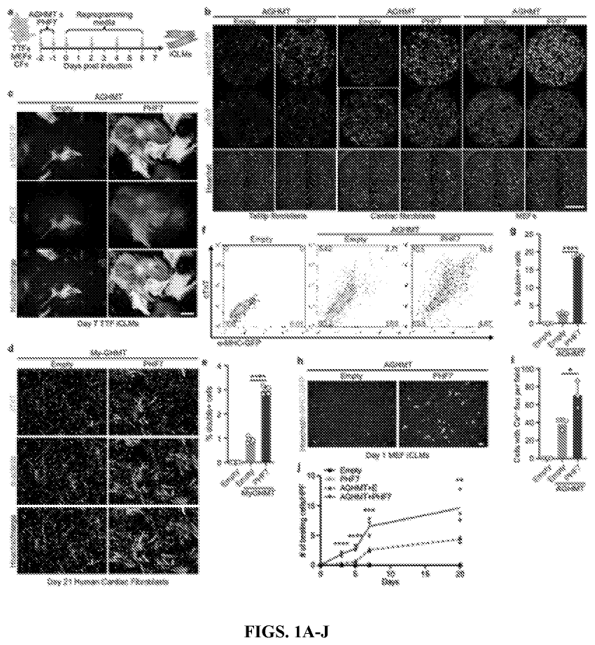

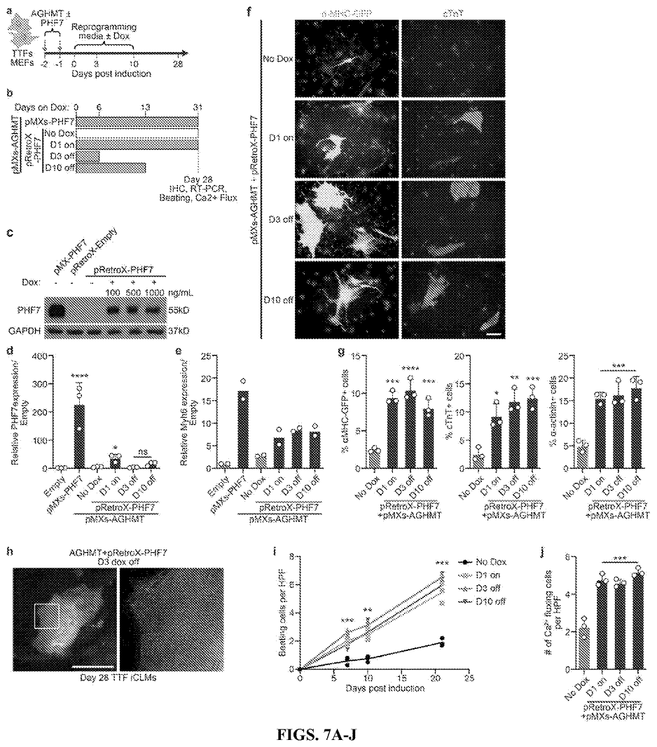

The inventors confirmed that PHF7 robustly activated reprogramming as measured by α-MHC-GFP and cardiac troponin T (cTnT) expression in TTFs derived from adult mice bearing an α-MHC-GFP transgene (FIGS. 1A-C, FIG. 6B). Importantly, PHF7 enhanced direct reprogramming in mouse CFs and embryonic fibroblasts (MEFs) as well as adult human CFs, confirming the cardiogenic potential of this factor (FIGS. 1B, 1D-E, FIGS. 6F-G). Flow cytometry demonstrated that addition of PHF7 to AGHMT-infected TTFs generated ˜20% double positive (cTnT+ and α-MHC-GFP+) iCLMs, representing a 10-fold increase in reprogramming efficiency from AGHMT alone (˜2% double positive cells) (FIGS. 1F-G, FIGS. 6C-D). Interestingly, robust activation of reprogramming in PHF7-treated cells was observed as early as 1 day following induction (FIG. 1H). Addition of PHF7 dramatically enriched cardiomyocyte markers, including Actc1, Myh6, and Tnni3 (FIG. 6E). Consistent with these findings, PHF7 increased the functional maturity of reprogrammed cells by accelerating and increasing spontaneously beating MEF iCLMs ˜2-fold and calcium flux by ˜1.5 fold (FIGS. 6I-J). Importantly, transient PHF7 expression achieved through a doxycycline-inducible system was sufficient to activate and maintain conversion of iCLMs, as evidenced by cardiac gene expression, sarcomere formation, spontaneous beating, and calcium flux FIGS. 7A-J).

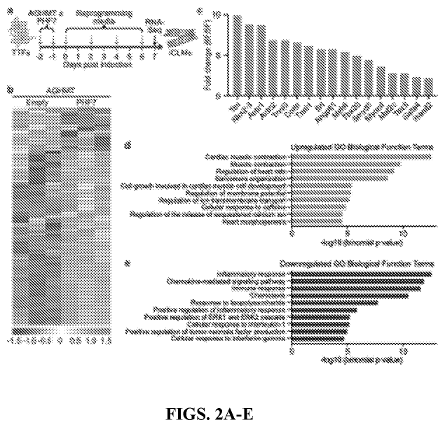

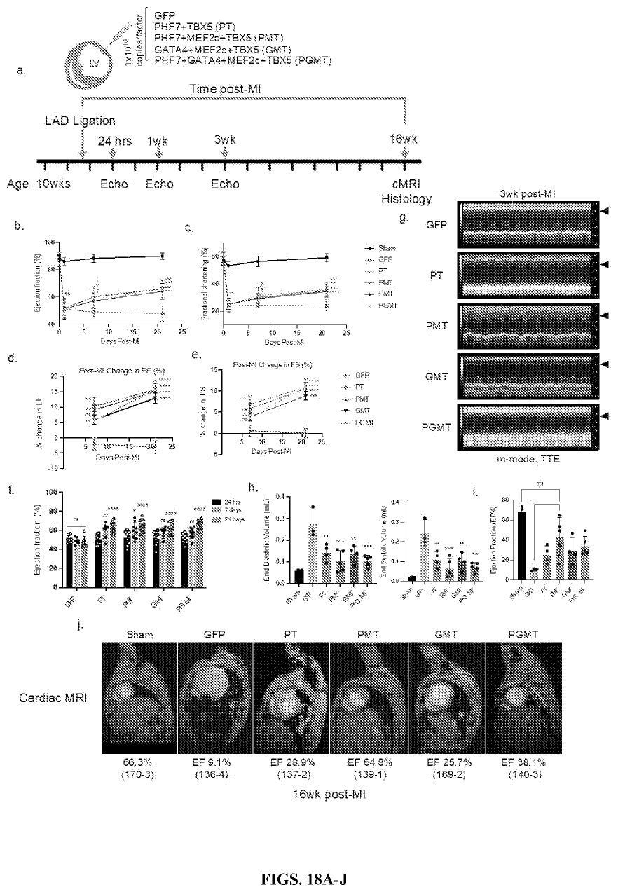

To decipher the effect of PHF7 on reprogramming globally, the inventors performed RNA sequencing using adult TTFs reprogrammed for 7 days with either AGHMT±Empty vector or AGHMT±PHF7 (FIG. 2A). PHF7 induced extensive transcriptomic changes, dysregulating expression of 737 genes, with ˜500 upregulated and ˜200 downregulated transcripts (FC>2, FDR≤0.01) (FIG. 2B). Amongst the genes most upregulated by PHF7 were those encoding mature cardiac structural proteins such as Ttn, Actc1, Actn2, Tcap, Tnni3, and Tnni1, as well as known critical cardiac developmental control genes such as Tbx20, Smyd1, and Myocd (FIG. 2C, FIGS. 18A-B). Interestingly, all four reprogramming TFs (Gata4, Hand2, Mej2c, Tbx5) were found to be significantly upregulated by PHF7 (FIG. 2C, FIGS. 18C-D). Examination of the 3′ untranslated regions (UTRs) of these factors confirmed that the increase in transcription originated from the endogenous loci (FIG. 18E). Gene ontology (GO) enrichment analysis revealed that PHF7 specifically upregulated genes associated with cardiac development and contractility (FIG. 2D). Downregulated GO terms were related to inflammatory, chemotactic, ERK, and TNF signaling pathways, which are known to present barriers to direct cell fate conversion (FIG. 2E). More specifically, gene set enrichment analyses (GSEA) revealed upregulation of myogenic targets and downregulation of E2F, Myc, G2M, and IL-6/Jak/Stat targets, which represent proliferative pathways known to be inhibitory to the reprogramming process (FIGS. 18F-G). However, EdU pulse labeling of TTF iCLMs at day 1 post-induction revealed no significant difference in the number of proliferating cells with the addition of PHF7, suggesting an alternative mechanism by which PHF7 potently activates reprogramming (FIGS. 18H-I).

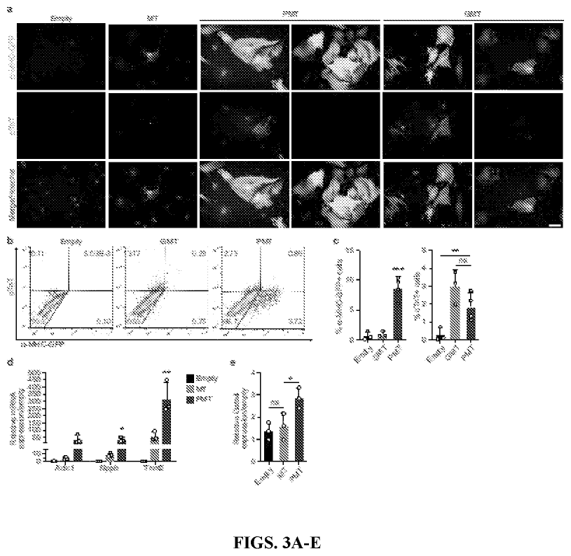

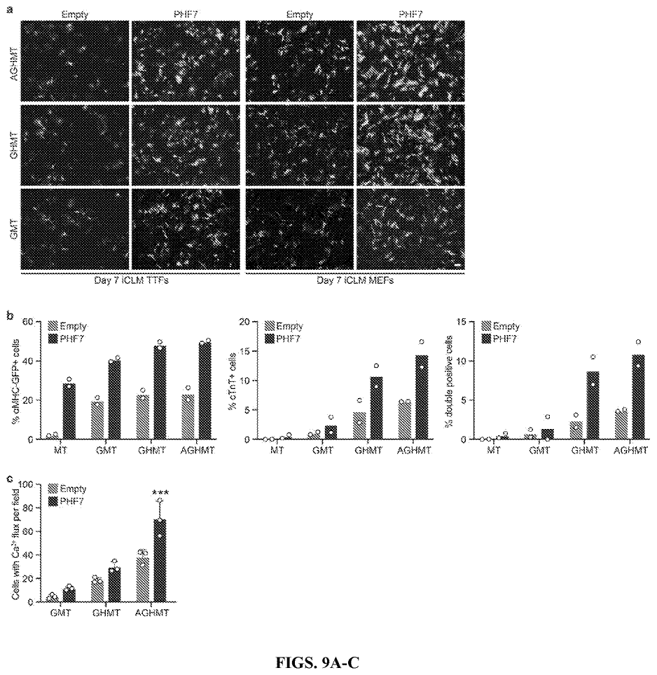

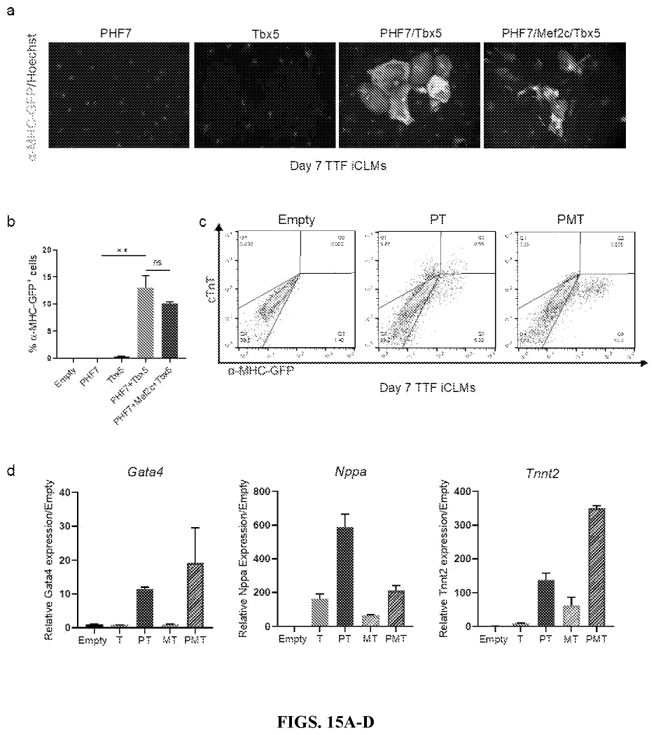

PHF7 achieves cardiac cell fate reprogramming in the absence of Gata4 and in the setting of minimal factors. Given the marked activation of the cardiac transcriptome with PHF7, the inventors hypothesized that PHF7 could activate reprogramming using fewer factors. Applying PHF7 or empty vector to both TTFs and MEFs in the presence of GMT, GHMT, or AGHMT, they observed profound induction of reprogramming by PHF7 in the context of all three cocktails (FIGS. 9A-C). The inventors then tested PHF7 with various combinations of factors and found that PHF7 significantly induced reprogramming when overexpressed in the absence of Gata4, with Mef2c and Tbx5 alone (FIG. 3A). This PHF7-Mef2c-Tbx5 cocktail, herein referred to as PMT, generated a substantial α-MHC-GFP⁺ population (˜10-20% of total cells) at 7 days post-induction, which was confirmed by both immunocytochemistry and flow cytometry (FIGS. 3B-C, FIG. 9B). Addition of PHF7 to Mef2c and Tbx5 induced cTnT expression as demonstrated by immunocytochemistry, flow cytometry, and qPCR (FIGS. 3A-D). Further, cardiac transcripts Actc1 and Nppb were enriched in the presence of PHF7 (FIG. 3D). Interestingly, PHF7 when added to Mef2c and Tbx5 similarly increased transcription of endogenous Gata4 as was observed following addition of PHF7 to AGHMT (FIG. 3E). Finally, the PMT cocktail induced occasional spontaneous calcium sparks at 14 days post-induction, suggesting functional potential of these iCLMs. This is noteworthy as to the inventors' knowledge, PHF7 is the only epigenetic factor whose overexpression has been found to induce cardiac reprogramming in the absence of Gata4³. The inventors further tested PHF7 with single factors and found that PHF7 was able to activate reprogramming in the presence of Tbx5 alone, inducing a population of ˜10% reprogrammed cells as demonstrated by immunocytochemistry quantification and flow cytometry studies (FIGS. 15A-C). Further, the addition of PHF7 to Tbx5 markedly upregulated several cardiac transcripts, including Gata4, Nppa, and Tnnt2, at 7 days post induction (FIG. 15D).

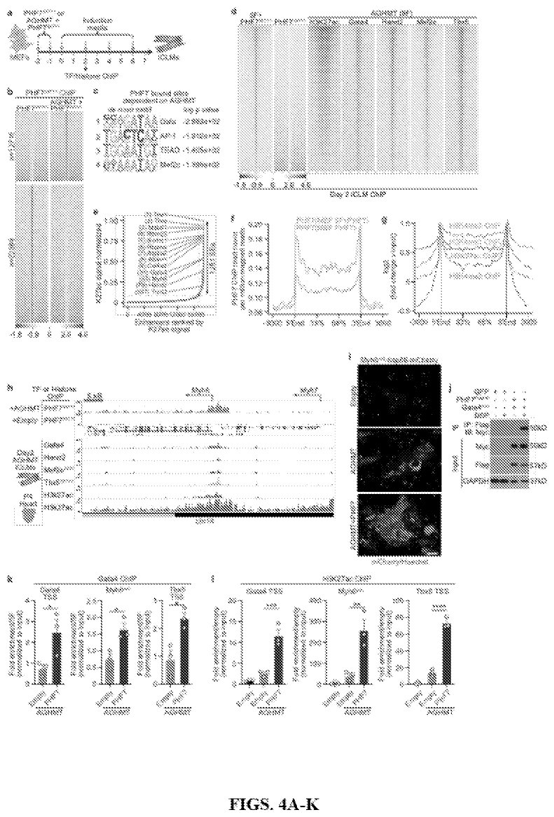

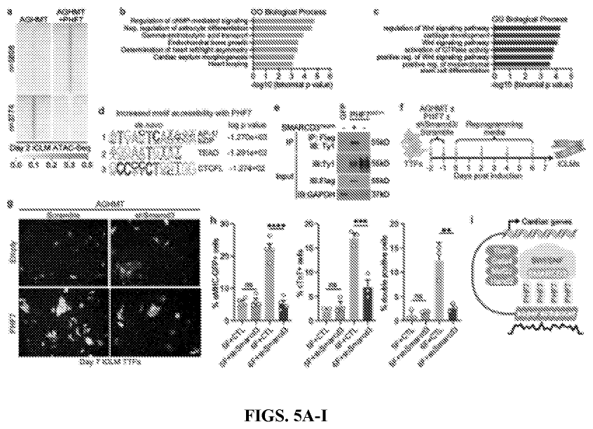

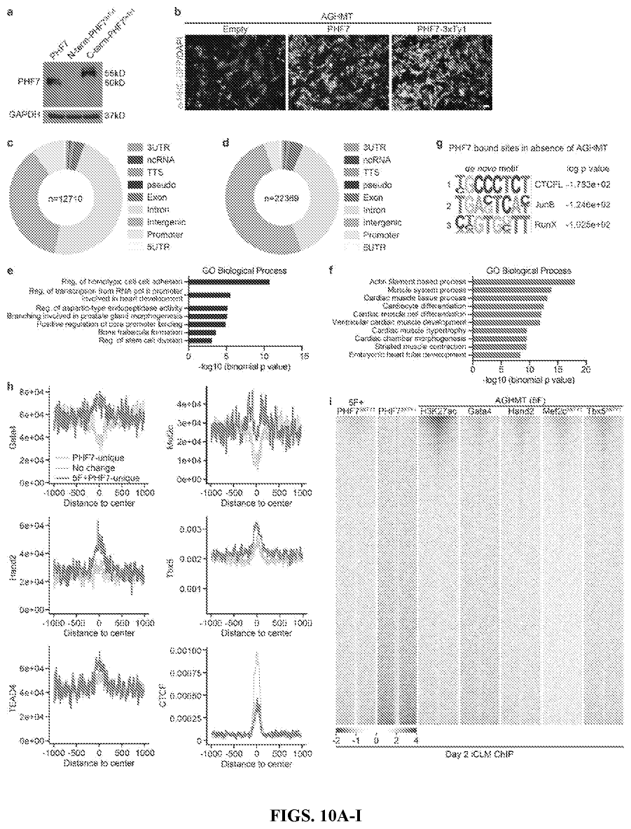

PHF7 globally co-occupies cardiac enhancers with cardiac TFs. To define the mechanism whereby PHF7 augments reprogramming, the inventors identified genome-wide binding sites of PHF7 using chromatin immunoprecipitation (ChIP) followed by massively paralleled deep sequencing (ChIP-Seq). MEFs were infected with epitope-tagged PHF7^{3xTy1} alone or with AGHMT reprogramming factors (AGHMT±PHF7^{3xTy1}) and harvested 2 days following induction for ChIP (FIG. 4A, FIGS. 10A-B). Through differential peak analysis, the inventors found that PHF7 occupied dramatically different genomic loci in the setting of reprogramming factors, with 12,710 peaks present and 22,369 peaks absent in the setting of AGHMT (FIG. 4B). In both conditions, PHF7 bound to predominantly intergenic and intronic genomic regions, consistent with transcription factor binding patterns and accession of regulatory elements (Extended Data FIGS. 5c-d). Peaks enriched in the presence of PHF7 alone demonstrated GO pathways related to cell-cell adhesion and promoter binding processes, while motif analyses revealed enrichment of CTCFL, AP-1, and Runx motifs (FIGS. 10E, 10G). In the context of reprogramming factors, GO biological process terms demonstrated strong recruitment of PHF7 to genomic regions regulating cardiac development and function (FIG. 10F). Searching for motifs in these PHF7 reprogramming peaks revealed enrichment of Gata4, AP-1, TEAD, and Mef2 motifs (FIG. 4C). Evaluating motif frequency amongst peak clusters confirmed enrichment of Gata4, Mef2c, and Hand2 motifs specifically in PHF7 reprogramming peaks, implying substantial co-occupancy of these TFs with PHF7 during reprogramming (FIG. 10H).

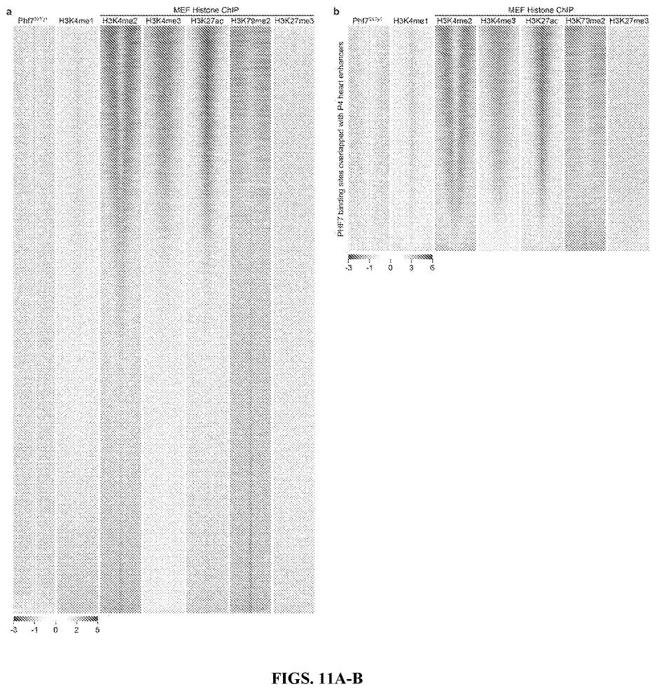

Given the results of motif enrichment analyses, the inventors interrogated the degree of genome-wide co-occupancy of PHF7 with reprogramming TFs in AGHMT iCLMs and observed remarkable co-localization of all four cardiac TFs at these PHF7 reprogramming peak regions (FIG. 4D, FIG. 10I) (Hashimoto et al., 2019). Observing the high fidelity binding of PHF7 to multivalent cardiac lineage-defining TF loci, the inventors hypothesized that PHF7 localized to cardiac super enhancers (SEs) and cooperated with cardiac TFs as a core transcriptional complex to dictate cardiac cell identity (Whyte et al., 2013). To address this hypothesis, they called SEs using H3K27ac ChIP obtained from the P4 mouse ventricle and annotated 1251 cardiac SEs, ranked by H3K27ac signal intensity (FIG. 4E, Supplementary Data Table 1). By mapping PHF7^{3xTy1} ChIP signal globally across cardiac SEs, the inventors observed PHF7 binding events at all 1251 SE regions, regardless of the presence of reprogramming factors (FIG. 4F, Supplementary Data Table 1). However, binding of PHF7 to cardiac SEs was significantly enriched in the presence of AGHMT (FIG. 4F). They hypothesized that PHF7 localized to these cardiac regulatory regions in fibroblasts through its known ability to read and directly bind H3K4me2 and H3K4me3 modifications (Yang et al., 2012; (Yang et al., 2012; Wang et al., 2019). To address this hypothesis, the inventors aligned histone ChIP in MEFs and found that PHF7 co-localized with H3K4me2, a modification that demarcates transcription factor binding sites (Wang et al., 2014; Chronis et al., 2017). PHF7 also aligned with activating modifications H3K4me3, H3K27ac, and H3K79me2, but was absent from sites with repressive H3K27me3 marks (FIG. 11A). Through mapping MEF histone ChIP signal globally across P4 heart enhancers, they found that H3K4me2 marked cardiac enhancers in MEFs, suggesting a mechanism by which PHF7 identifies cardiac regulatory regions in fibroblasts (FIG. 4G, FIG. 11B).

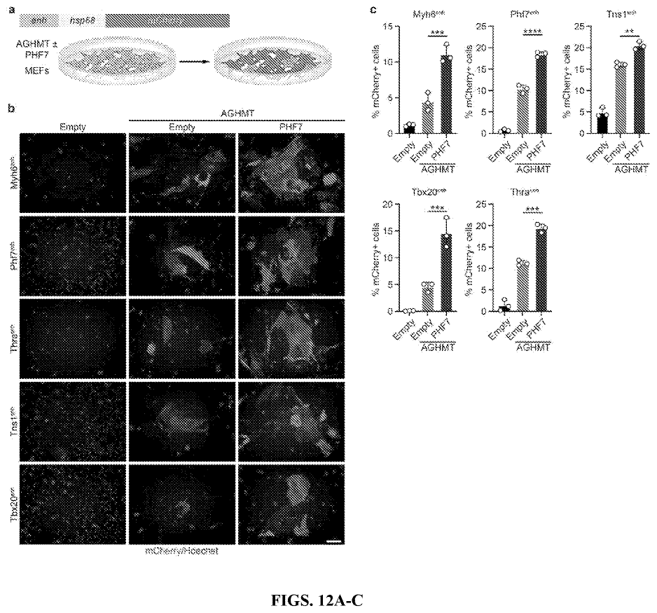

Interestingly, PHF7 binding was observed at the myosin heavy chain (Myh6/7) SE, a region known to govern cardiac myosin isoform switching and cardiac gene programs through SWI/SNF complex recruitment (FIGS. 4E, 4E) (Hang et al., 2010). The inventors identified multivalent cardiac TF binding and modest H3K27ac deposition at this region in the absence of PHF7 (FIG. 4H). Importantly, the addition of PHF7 markedly increased Myh6/Myh7 enhancer activation as measured by H3K27ac deposition, thus identifying a critical barrier through which PHF7 promotes reprogramming (FIG. 4I). The inventors then validated activation of the Myh6 enhancer (Myh6^{enh}) as well as other enhancers (Tns1^{enh}, Thra^{enh}, Tbx20^{enh}) identified from the in silico SE analysis through generation of enhancer-hsp68-mCherry retroviral constructs. Delivery of these reporter constructs with AGHMT induced mCherry expression, and addition of PHF7 further increased activation of these cardiac enhancer elements (FIG. 4I, FIGS. 12A-C).

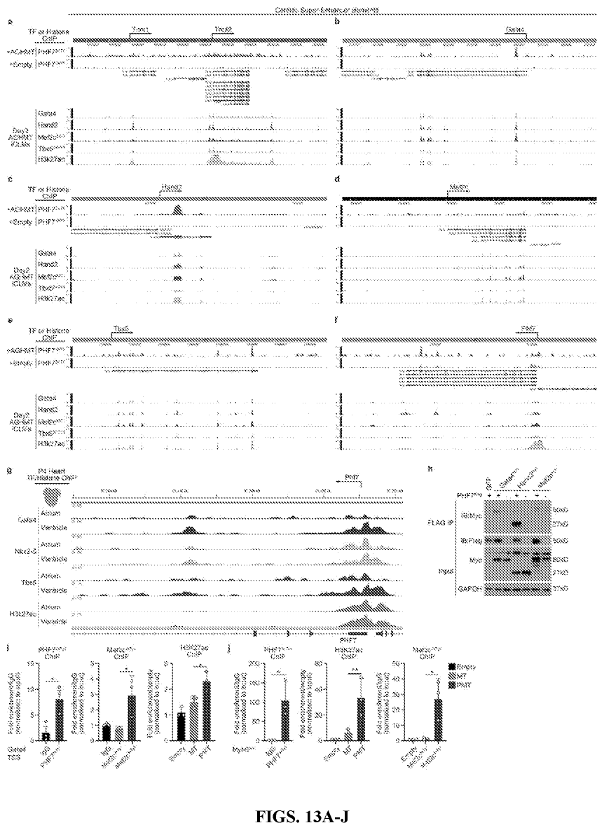

Previous studies demonstrated that Gata4, Hand2, Mef2c, and Tbx5 were core TFs that could bind to both self and one another at their respective SEs, creating a core transcriptional regulatory circuit (He et al., 2011; Saint-Andre et al., 2016). Interestingly, PHF7 and cardiac TF binding events were identified at SEs annotated to the core reprogramming TFs themselves (FIGS. 13A-E, Supplementary Data Table 1). Further, a mouse P4 cardiac SE was annotated to Phf7 (rank 331/1251), and the inventors identified PHF7 binding to its own SE at sites co-bound by cardiac TFs (FIG. 13F, Supplementary Data Table 1). TF ChIP from in vivo P4 mouse ventricle further identified endogenous Gata4, Tbx5, and Nkx2-5 binding throughout the Phf7 TSS and SE, suggesting the persistence of this regulatory network in vivo (FIG. 13G). Together, these ChIP and transcriptomic data suggest that PHF7 participates with Gata4, Hand2, Mef2c, and Tbx5 in a cardiac TF auto-regulatory circuit.

To validate PHF7 co-occupancy with cardiac reprogramming factors, the inventors performed immunoprecipitation assays to examine interaction between PHF7 and the cardiac TFs. They observed strong interaction between PHF7^{FLAG-HA} and Gata4^{myc}, Hand2^{myc}, and Mef2c^{myc} by Flag IP (FIG. 4J, FIG. 13H). Given these interactions, the inventors hypothesized that PHF7 impacts binding of these cardiac TFs to cardiac enhancers. Through Gata4 ChIP, they found that PHF7 strengthened the binding of Gata4 to both itself and its targets, thus reinforcing the observed positive autoregulatory circuit (FIG. 4D). Further, PHF7 activated these cardiac enhancers as demonstrated by H3K27ac ChIP (FIG. 4I).

The inventors hypothesized that these mechanisms were maintained in the absence of Gata4 and could account for the enhanced reprogramming efficiency observed using the PMT cocktail. To explore this hypothesis, they performed PHF7^{3xTy1} ChIP in the context of reprogramming with PHF7^{3xTy1}, Mef2c, and Tbx5 (PMT) and identified strong binding of PHF7^{3xTy1} to the Myh6 enhancer (Myh6^{enh}) and Gata4 TSS (FIGS. 13I-J). As the inventors had observed interaction between Mef2c and PHF7, they hypothesized that PHF7 also impacts the strength of Mef2c binding to its targets. By Mef2c^{3xTy1} ChIP, they found that PHF7 significantly strengthened binding of Mef2c^{3xTy1} to Myh6^{enh} and Gata4 TSS in the context of PMT compared to Mef2c and Tbx5 (MT) alone (FIGS. 13I-Jj). Addition of PHF7 to MT markedly increased deposition of active enhancer marks at the Myh6 and Gata4 locus as determined by H3K27ac ChIP (FIGS. 13I-J). These data suggest that PHF7 promotes activation of cardiac enhancers, increases cardiac TF binding, and activates a core cardiac autoregulatory circuit in the absence of the pioneer factor Gata4.

PHF7 interacts with SMARCD3/BAF60c to promote reprogramming. To assess global changes in chromatin accessibility, the inventors performed ATAC-seq in day 2 AGHMT MEF iCLMs in the presence and absence of PHF7. PHF7 induced broad changes in chromatin architecture, yielding 5808 peaks with increased chromatin accessibility, and 3774 peaks with decreased accessibility (FC>2) (FIG. 5A). By GO pathway analysis, open chromatin peaks were annotated to regions regulating cAMP-mediated signaling and cardiac morphogenesis (FIG. 5B). These accessed regions were enriched for AP-1, CTCF, and TEAD motifs by de novo motif analysis (FIG. 5D). Interestingly, closed chromatin peaks were related to Wnt signaling, a pathway prohibitive to direct reprogramming (FIG. 5C).

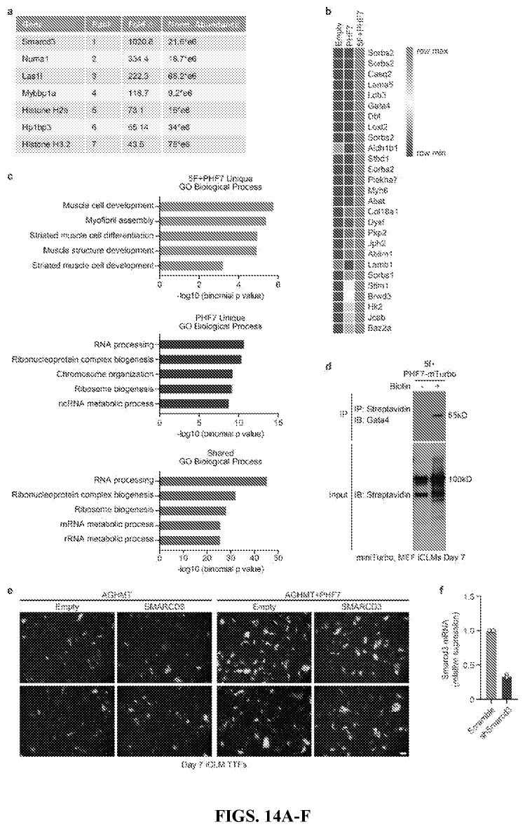

Given the ability of PHF7 to modify chromatin structure, the inventors hypothesized that PHF7 recruits remodeling complexes to enact changes in accessibility. To identify interacting partners, the inventors utilized a miniTurbo biotinylation-based proximity ligation assay, infecting MEFs with either PHF7^{mTurbo} or AGHMT±PHF7^{mTurbo}. Amongst proteomics hits shared between PHF7^{mTurbo} and AGHMT±PHF7^{mTurbo} conditions, they identified several histones (Histone H2a, Histone H3.2) as well as heterochromatin binding proteins (Hp1bp3, Mybbp1a, and Las11) (FIG. 14A, Supplementary Data Table 2). However, the top hit in both conditions was Smarcd3/BAF60c, a SWI/SNF complex subunit known to orchestrate cardiac development and disrupt nucleosomal stability to regulate cardiac TF binding, proliferation, and Wnt signaling (Lickert et al., 2004; Devine et al., 2014; Takeuchi et al., 2007). Through FLAG co-IP, the inventors observed strong interaction between PHF7^{3xTy1} and SMARCD3^{FLAG-HA} (FIG. 5E). Shared hits were enriched in ribonucleoprotein complex factors, while interacting proteins unique to reprogramming conditions were almost exclusively cardiac and muscle-specific factors and included the reprogramming factor Gata4 (FIGS. 14B-D, Supplementary Data Table 2). These results are consistent with the known ability of Smarcd3 to recruit and interact with Gata4 and other cardiac TFs (Lickert et al., 2004; Takeuchi & Bruneau 2009).

The inventors were interested in whether the observed interaction between PHF7 and Smarcd3 was a causal mediator of the reprogramming phenotype. Forced overexpression of retroviral SMARCD3 in AGHMT reprogramming neither phenocopied PHF7 nor augmented its effect (FIG. 14E). Further, knockdown of Smarcd3 in AGHMT TTF reprogramming did not decrease reprogramming efficiency (FIGS. 5G-H, FIG. 14F). Remarkably however, when shSmarcd3 was applied to AGHMT+PHF7 reprogramming, knockdown of Smarcd3 significantly attenuated the effect of PHF7, yielding a reprogramming efficiency closer to that of AGHMT (FIGS. 5G-H). These findings suggest that Smarcd3 utilizes a histone reader such as PHF7 to identify cardiogenic sites and exert its chromatin modifying effects in fibroblasts (FIG. 5I).

The inventors further tested PHF7 with single factors and found that PHF7 was able to activate reprogramming in the presence of Tbx5 alone, inducing a population of ˜10% reprogrammed cells as demonstrated by immunocytochemistry quantification and flow cytometry studies (FIGS. 10A-D). Further, the addition of PHF7 to Tbx5 markedly upgregulated several cardiac transcripts, including Gata4, Nppa, and Tnnt2, at 7 days post induction.

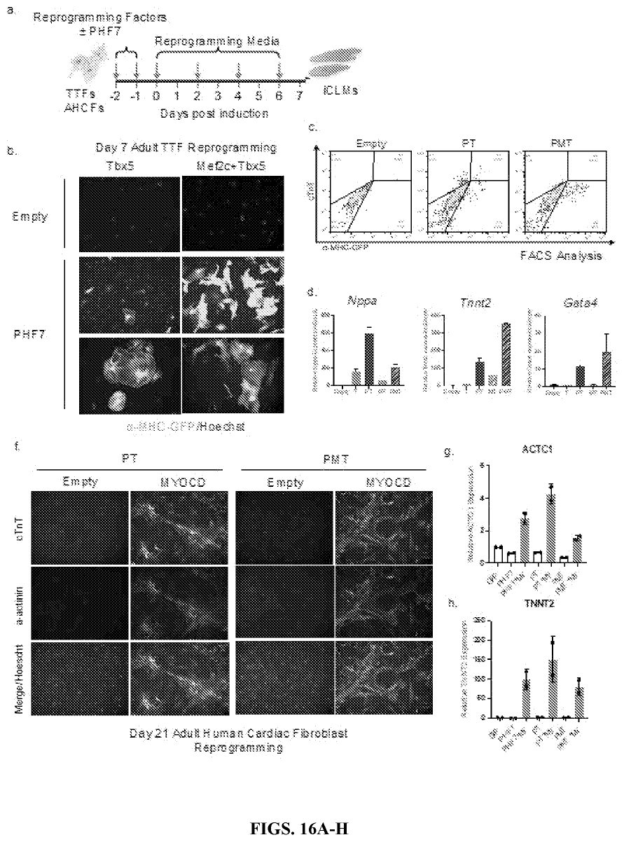

PHF7 activates reprogramming with TBX5 alone. Given the ability of PHF7 to activate reprogramming in the absence of Gata4, with Mef2c and Tbx5 alone (PMT), the inventors tested PHF7 with various combinations of factors, including single co-factors or TFs. They found that PHF7 was able to activate reprogramming in the presence of Tbx5 alone, inducing a population of ˜10% reprogrammed cells as demonstrated by immunocytochemistry flow cytometry studies (FIG. 16A-C). Further, the addition of PHF7 to Tbx5 markedly upregulated several cardiac transcripts, including Gata4, Nppa, and Tnnt2, at 7 days post induction (FIG. 16D). The inventors tested this finding in the context of reprogramming of adult human cardiac fibroblasts and found that PT and PMT cocktails, in the context of Myocardin (PT+Myocd or PMT+Myocd) were sufficient to activate adult human reprogramming and induce cardiac gene expression as measured by immunocytochemistry for the cardiac markers cTnT and α-actinin and real-time PCR, respectively (FIGS. 16E-H). This is a landmark finding as a three-factor cocktail has not been previously shown in the literature to active adult human cardiac reprogramming.

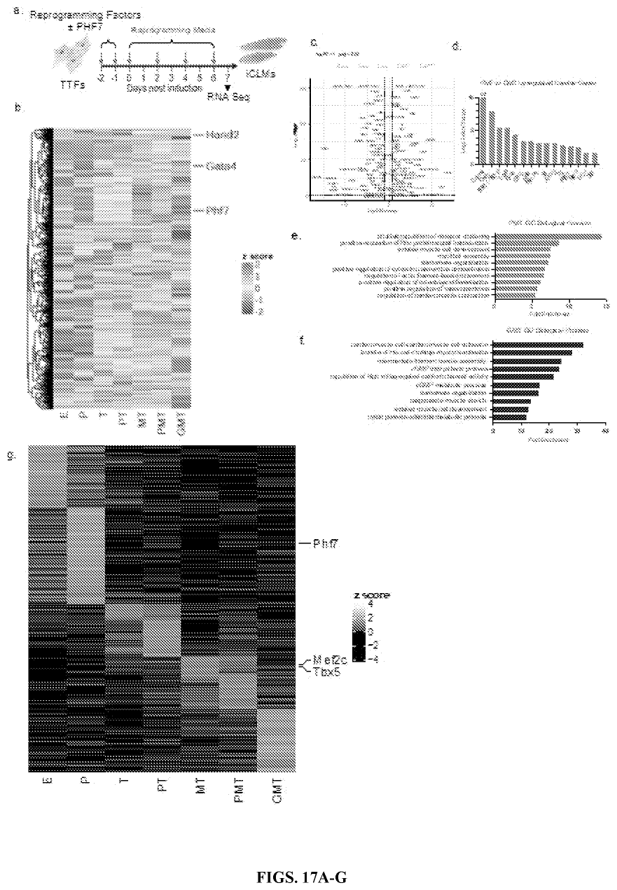

PHF7 cocktails activate a global but distinct cardiac transcriptome. To decipher the global impact of minimalist PHF7 cocktails on reprogramming, the inventors performed RNA sequencing using adult TTFs reprogrammed for 7 days with PT, PMT, or GMT, along with appropriate controls. (FIG. 17A). PHF7 and its cocktails induced extensive transcriptomic changes, with each cocktail upregulating its own distinctive gene program, when compared to controls (FC>2, FDR≤0.01) (FIG. 17B). Comparing PMT to GMT, there were over 1000 differentially expressed genes, with 590 transcripts upregulated in PMT and 460 transcripts that were downregulated in the GMT-treated iCLMs (FIG. 17C). Amongst the genes most upregulated by PHF7 were those encoding mature cardiac structural proteins such as Calm4, Cntn2, Tcap, Gjd4, Srl, Des, Nebl, as well as known critical cardiac developmental control genes such as Smyd1/BOP, Esrrg, Gata4, Tg, and Xirp2 (FIG. 17D). Gene ontology (GO) enrichment analysis revealed that PHF7 specifically upregulated genes associated with cardiac development and contractility (FIG. 17E). Transcripts that were differentially upregulated by the GMT cocktail were related to cardiac muscle cell adhesion and structure and cGMP metabolic (FIG. 17F). Importantly, iCLMs transduced with each of these cocktails possess a unique gene signature from both their controls and one another (FIG. 17G).

PHF7 cocktails improve cardiac function following myocardial infarction. The ultimate indicator of therapeutic potential for a reprogramming cocktail is its ability to reprogram adult human cardiac fibroblasts and rescue cardiac function following myocardial infarction (MI). While numerous factors including transcription factors, kinases, small molecules, shRNAs, and miRNAs, have been proposed to activate reprogramming in embryonic or postnatal cell types, few factors have demonstrated benefit in terminally differentiated adult mouse and human cells, as seen with PHF7. To the inventors' knowledge, PHF7 is the first factor to activate adult reprogramming in the presence of a single factor. Thus, the inventors were keenly interested in confirming the cardiogenic potential of PHF7 in vivo following ischemic injury. They induced myocardial infarction through permanent LAD ligation on C57/BL6 wild-type mice and injected concentrated retroviruses encoding the PT (PHF7+Tbx5), PMT (PHF7+Mef2c+Tbx5), GMT (Gata4+Mef2c+Tbx5), PHF7+GMT, or GFP control cocktail into the border zone immediately following LAD ligation (FIG. 18A) Sham controls injected with either retroviral treatment or control cocktail were used for all treatment groups. Echocardiography was then performed at 24-hours, 1 week, and 4 weeks post-MI to assess degree of surgically-induced injury as well as change in general cardiac function parameters including fractional shortening, ejection fraction, and end systolic and diastolic dimensions between all groups (FIG. 18A). The inventors confirmed that all animals in the LAD groups experienced the same degree of myocardial injury at 24 hours post-MI (FIGS. 18B-C). They discovered that PHF7 cocktails induced improvement in cardiac function as soon as 7 days post-MI, and continued to improve at 21 days post-MI, as measured by ejection fraction and fractional shortening (FIGS. 18B-C). Measuring the degree improvement in cardiac function following MI, PHF7-cocktails induced a 10-15% increase in ejection fraction at 7 days post-MI compared to GFP controls that increased to a 15-20% increase at day 21 (FIGS. 18D and 18F) This degree of improvement was similarly seen for PT, PMT, GMT, and P+GMT cocktails through calculation of fractional shortening (FIG. 18E). Representative m-mode images from transthoracic echo are shown at 3 weeks post-MI demonstrating this improvement in the function of the infarcted anterior wall (FIG. 18G). The gold standard for determination of ejection fraction and degree of scar following myocardial in both the human and mouse heart is cardiac MRI with the use of gadolinium. The inventors performed cardiac MRI on n=3-5 mice per group at 16 weeks post-MI to assess the ejection fraction and chamber sizes of Sham and LAD groups treated with the aforementioned cocktails. Outliers in ejection fraction for all of the groups were included to confirm the reliability of the echocardiographic measurements. They found that there was a statistically significant decrease in End Diastolic Volume (EDV) in PT, PMT, GMT, and P+GMT treated hearts compared to GFP control treated hearts, suggesting positive remodeling of the LV cavity following myocardial infarction (U FIG. 18H). Further, there was a similar decrease in End systolic volume (ESV) in treated hearts compared to controls, with the most significant statistical change being seen in PMT and P+GMT treated hearts compared to GFP controls (FIG. 18H). Further, there was a statistically significant increase in the ejection fraction of PMT treated hearts compared to GFP controls (FIG. 18I). These data included outliers for the importance of corroborating the echocardiographic findings, likely diminishing the statistical significance of other treatment groups, where a strong trend toward statistical significance was certainly noted. Representative images of short axis T2 weighted imaging are shown demonstrating the large degree of cardiac remodeling and remuscularization in the PHF7-treated hearts compared to GFP-treated hearts.

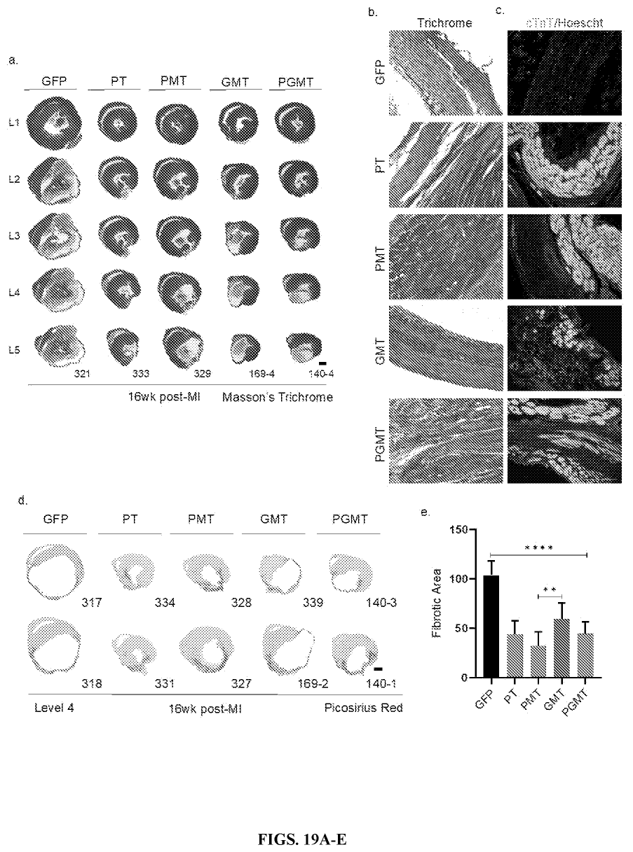

PHF7 decreases fibrotic scar and induces remuscularization following myocardial infarction. To evaluate the degree of fibrosis following reprogramming with PHF7 cocktails, the inventors harvested all hearts that had undergone Sham surgery or LAD ligation at 16 weeks post-MI and performed Masson's Trichrome staining on all tissues (FIG. 19A). By trichrome staining, there was clear remodeling, decreased fibrotic scar, and increased remuscularization present in the PT, PMT, and P+GMT treated hearts as well as GMT, which was used as a positive control (FIGS. 19A-B). This remuscularization was demonstrated by troponin staining of the infarcted region, which demonstrated an increase in troponin positive cells within the scarred area for all PHF7-treated hearts (FIG. 19C). The marked improvement in the degree of fibrosis and scar in PHF7-treated groups was again demonstrated with a different modality, using Picosirius red staining (PSR) (FIG. 19D). The degree of fibrosis was calculated using ImageJ applying a mask over whole tissue sections to assess scar area in a standardized fashion. Every heart (n=8-9 per group) that had undergone surgery was included in the calculations. All cocktails demonstrated a statistically significant decrease in scar area compared to GFP control, however PMT-treated hearts had a statistically smaller scar area than GMT-treated hearts, while other PHF7 cocktails trended toward similar statistical superiority relative to GMT (FIG. 19E). These data demonstrate that PHF7-based cocktails containing minimal other co-factors, and lacking Gata4 are sufficient, and in some outcomes statistically superior to GMT when evaluating chronic changes in cardiac function, remuscularization, and scar area over time.

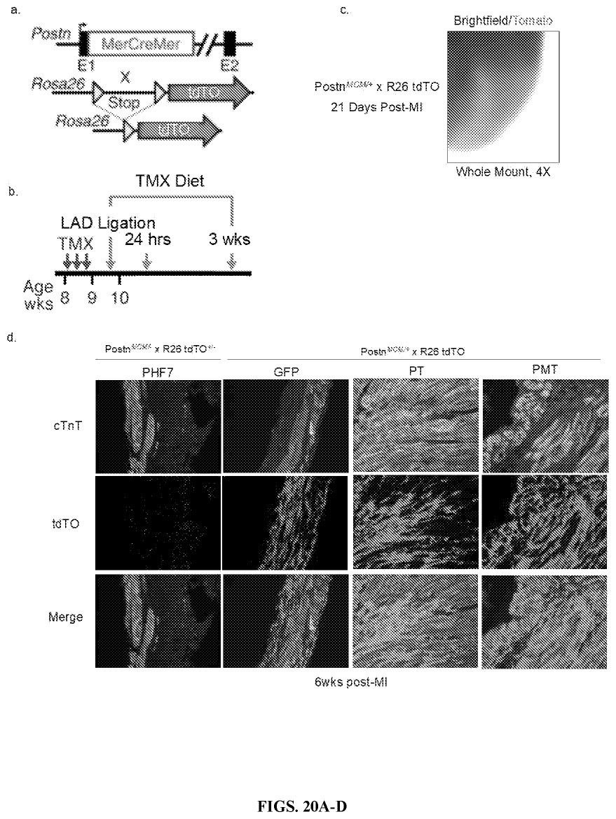

PHF7 induces direct reprogramming of fibroblasts to cardiomyocytes following myocardial infarction. To confirm that changes in cardiac function are a result of directed fibroblast to reprogramming, the inventors performed lineage tracing of fibroblasts using a well-studied inducible genetic inducible lineage tracing model that labels periostin (Postn), a specific marker of injury-activated fibroblasts. The inventors performed LAD ligation on Postn^{MCM+} mice crossed to Rosa26-tdTO mice induced on tamoxifen chow (FIGS. 20A-B). They first confirmed that this strategy activated tdTO expression specifically in non-myocytes of the infarct zone as previously described (FIG. 20C). The inventors then undertook experimental studies, injecting concentrated retroviruses encoding the PT, PMT, or GFP control cocktail into the border zone immediately following LAD ligation (n=4-5/group). They then analyzed tdTomato, cTnT, and α-actinin expression in histological sections of hearts at 6 weeks post-MI. As previously described, there was no leakiness of the Cre visualized in tamoxifen-treated Postn^{MCM/-}; R26-tdTO mice (FIG. 20D). Cre positive GFP-control sections demonstrated marked staining of thin cells within the scar and intercalated in between myocytes of the peri-infarct area; there was no evident overlap between cTnT and tdTO staining in GFP-treated sections (FIG. 20D). However, in PT and PMT treated sections, there were a large number of cTnT+/tdTO+ myocytes. While some of the myocytes appeared smaller and more immature than the healthy myocytes of the non-injured portion of the tissue, others had clear and complete sarcomere definition (FIG. 20D). While these data do not exclude the possibility of other non-myocytes contributing to the total reprogrammed cell population, they indicate that PHF7 is in fact directly reprogramming fibroblasts to cTnT+ and α-actinin+ myocytes.

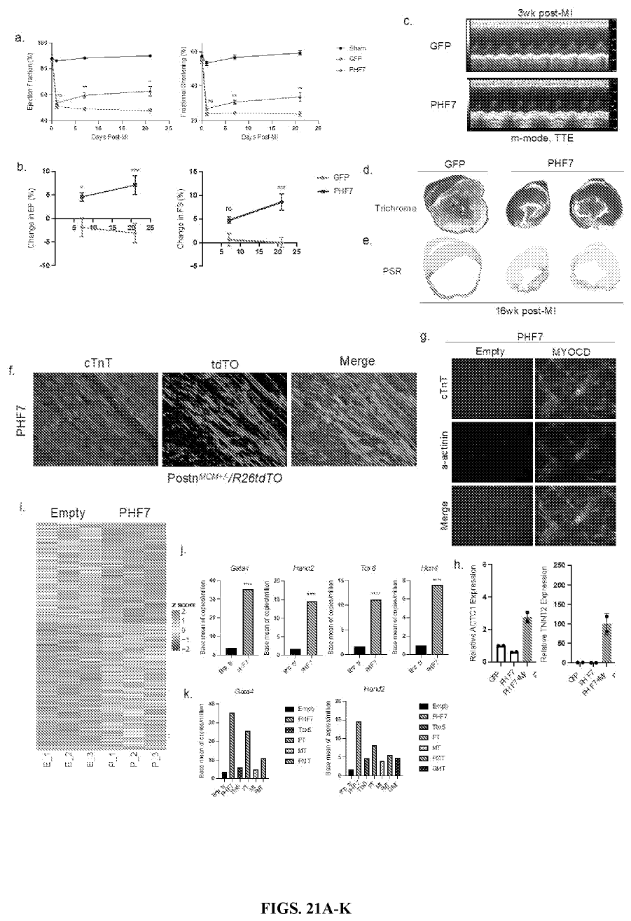

A single factor, PHF7, improves cardiac function through reprogramming following injury. No single factor in isolation has been shown either induce reprogramming in vitro or induce reprogramming in vivo following myocardial infarction. Given the data demonstrating the ability of PHF7 to recognize cardiac super enhancers when overexpressed in isolation in fibroblasts, the inventors hypothesized that PHF7, within the appropriate niche, may be able to achieve fibroblast to cardiomyocyte reprogramming in vivo. They therefore induced myocardial infarction through permanent LAD ligation on C57/BL6 wild-type mice and injected concentrated retroviruses encoding GFP (control) or PHF7 into the border zone immediately following LAD ligation, as was done for other subjects in this manuscript. Sham controls were used for all treatment groups. Echocardiography was similarly performed at 24-hours, 1 week, and 3 weeks post-MI to assess degree of surgically-induced injury as well as change in general cardiac function parameters including fractional shortening, ejection fraction, and end systolic and diastolic dimensions between all groups (FIG. 21A). The inventors again confirmed that all animals in the LAD groups experienced the same degree of myocardial injury at 24 hours post-MI (FIG. 21A). Through echocardiography, they discovered that injection of PHF7 alone was sufficient to improve cardiac function post-MI (FIG. 21A-B). While GFP-treated mice experienced progressive decline in their ejection fraction, PHF7 was sufficient to not just stabilize, but improve cardiac function as measured by both ejection fraction and fractional shortening. Measuring the degree improvement in cardiac function following MI, PHF7 when injected in isolation induced a 5-10% increase in ejection fraction at 7 days post-MI when compared to GFP controls. This continued to increase to a 10-15% increase in ejection fraction and fractional shortening by day 21 post-MI (FIG. 21B). This is demonstrated by representative m-mode images demonstrating contractile recovery of the infarcted anterior wall in PHF7-treated heart compared to the GFP-treated heart (FIG. 21C). The inventors maintained these mice to 16 weeks post-MI and harvested their hearts for histology. Masson's Trichrome and Picosirius Red staining of GFP and PHF7-treated sections demonstrate the improvement in remodeling, decreased scar area, and remuscularization of the PHF7-treated hearts. They confirmed that this benefit was indeed occurring through direct reprogramming by performing lineage tracing on Postn^{MCM/-}; R26-tdTO mice injected with PHF7 and identified large numbers of cTnT+/tdTO+ myocytes in the infarct and peri-infarct region (FIG. 21F). Given this intriguing finding, the inventors returned to their model of adult human reprogramming to assess the ability of PHF7 to induce reprogramming in the absence of excess factors. Very few cocktails have demonstrated the ability to reprogram human cells in the absence of Myocardin (MYOCD). Therefore, they applied PHF7 with and without MYOCD to adult human cardiac fibroblasts and induced reprogramming. The inventors found that PHF7, in the presence of myocardin was sufficient to induce reprogramming as measured by cTnT and a-actinin expression on both the transcript and protein level at 21 days post-induction (FIGS. 21G-H). In search of further mechanistic cues beyond that which they had previously described, the inventors returned to their batch RNA-Sequencing data comparing PHF7 to Empty controls and evaluated the differentially expressed genes (FIG. 21I). While there were no statistically significant GO terms between the differentially regulated genes, a number of transcription factors known to be critical to cardiac development and mesodermal cell fate specification were markedly upregulated by PHF7. Interestingly, the reprogramming TFs, Gata4 and Hand2, were found to be significantly upregulated by PHF7 (FIGS. 21J-K), as well as Tbx6, a master regulator of mesoderm cell fate specification and Hcn4. The inventors demonstrated again as previously described that Gata4 was upregulated 2-3 fold in the setting of PMT cocktail compared to control, however in the setting of PHF7 overexpression alone, Gata4 is induced over 30-fold (FIG. 21K). Similarly, Hand2 is activated over 15-fold compared to control, and its activation declines with the presence of additional factors (FIG. 21K). This intriguing finding is likely the result of the positive transcriptional autoregulatory circuit activated by PHF7 that the inventors previously discovered and described in this patent.

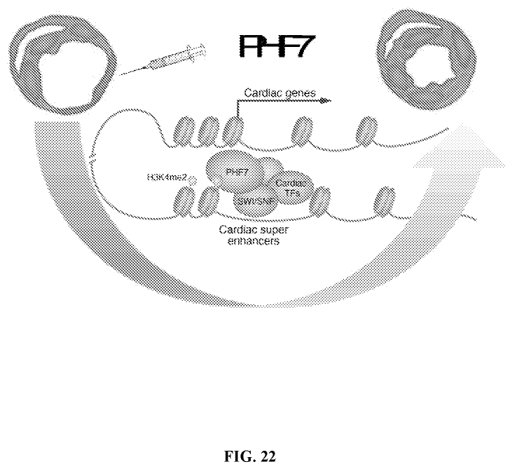

PHF7 as a single factor and within minimalist transcription factor cocktails improve cardiac function post-MI via direct fibroblast to cardiomyocyte reprogramming. These data describe the ability of PHF7, in the presence of minimal co-factors, to induce adult human cardiac fibroblast reprogramming in vitro. Further, these data demonstrate the ability of PHF7 cocktails (PHF7, PT, PMT, P+GMT) to significantly improve cardiac function following myocardial infarction compared to controls, and by some outcomes, demonstrate statistical superiority (as opposed to mere non-inferiority) to the previously utilized and well-studied GMT. It should be noted that GMT has not been shown to be efficacious in adult human cardiac reprogramming. The inventors then demonstrate the ability of PHF7 in isolation to improve cardiac function and decrease scar area when injected following myocardial infarction. They demonstrate for all PHF7-based cocktails that PHF7 indeed induces direct reprogramming of activated fibroblasts to cardiomyocytes in vivo (FIG. 22). Lastly, they demonstrate the ability of PHF7 with myocardin to induce adult human reprogramming and define PHF7 alone as a regulator of cardiac TF expression in fibroblasts.

### Example 3—Discussion

While significant attention has been focused on transcription factor biology in cardiac reprogramming, the field's understanding of and ability to harness epigenetic regulatory mechanisms remain in their infancy. Thus far, few epigenetic regulators have been shown to impact direct reprogramming. Knockdown of the polycomb complex protein Bmi1 is known to enhance reprogramming and permit cell fate conversion in the presence of Mef2c and Tbx5¹⁹. Overexpression of Smarcd3/BAF60c alongside Gata4 and Tbx5 is critical in mesodermal differentiation to cardiomyocyte-like cells (Takeuchi & Bruneau 2009). However, overexpression of SWI/SNF and other cardiac-specific epigenetic regulatory complexes have shown limited or no benefit in fibroblast reprogramming (Christoforou et al., 2013; Zhou et al., 2017).

In these studies, the inventors define the histone reader PHF7 as a critical factor in overcoming barriers of direct reprogramming through its ability to stabilize TF binding at cardiac enhancers. Interestingly, PHF7 recognized cardiac SEs in fibroblasts in the absence of reprogramming factors, suggesting that this reader is involved in the earliest steps of cardiac enhancer recognition and activation. Notably, the addition of reprogramming factors greatly enhanced recruitment of PHF7 to cardiac SEs. Through cooperation with the SWI/SNF complex, PHF7 increased chromatin accessibility at these SEs, thereby informing expression of cardiac gene programs. Further, PHF7 participated in and activated a cardiac TF autoregulatory circuit, regulating endogenous transcription of the core TFs themselves. Consistent with its function as a histone reader recruiting the SWI/SNF complex, PHF7 is dependent on endogenous Smarcd3 expression for its phenotype in reprogramming. Conceivably, PHF7 may facilitate SWI/SNF eviction of repressive PRC1 complexes from cardiac enhancers, potentially linking the mechanisms observed with Bmi1 knockdown and PHF7 overexpression and is a hypothesis deserving of further investigation (Zhou et al., 2016; Stanton et al., 2017).

Minimizing factors for therapeutic administration has been of great interest to the reprogramming field. Importantly, the inventors show that PHF7 enhances efficiency of all TF cocktails and accomplishes reprogramming in the absence of the pioneer factor Gata4. In this setting, PHF7 not only strongly strengthened Mef2c binding to its targets, but also recruited Mef2c to induce activation of the TF autoregulatory circuit in the absence of exogenous Gata4 (Hashimoto et al., 2019). Through these mechanisms, PHF7 rapidly overcomes epigenetic barriers to reprogramming at cardiac regulatory elements that added TFs fail to efficiently achieve on their own. Further, the inventors identified that PHF7 was capable of inducing reprogramming and cardiac gene expression in vitro in the presence of Tbx5 alone, establishing its role as one of the most robust and comprehensive reprogramming factors to date. The authors further identified that in the setting of adult human cardiac fibroblast reprogramming, that PHF7 and myocardin alone were capable of inducing cardiac reprogramming. Lastly, the authors showed that PHF7, when overexpressed in isolation in fibroblasts, induces massive activation of Gata4, Hand2, and Tbx6 expression, again demonstrating that PHF7 is on its own capable of activating a TF autoregulatory circuit in the absence of other exogenous factors.

When these minimalist cocktails were tested in models of acute injury post-myocardial infarction, all tested PHF7 cocktails (PHF7 and TBX5, PHF7, MEF2C, and TBX5, and PHF7, GATA4, MEF2C, and TBX5) were shown to improve cardiac function, induce cardiac remodeling, decrease fibrosis, reverse heart failure, and improve mortality. Further, when PHF7 alone was delivered to the injured myocardium post-MI, PHF7 induced an increase in cardiac function, increased cardiac remodeling, decreased fibrosis, reversed heart failure, and improved mortality compared to controls. These studies were carried out over 4 months post-MI and demonstrate a positive impact on chronic heart failure. We further show that this impact was achieved through cardiac reprogramming of fibroblasts to cardiac-like myocytes (iCMs) using in vivo lineage tracing models. For the first time, we show a single factor, PHF7, capable of improving cardiac function post-MI through direct cardiac reprogramming.

Direct cardiac reprogramming has rich potential for therapeutic translation, but numerous roadblocks persist, the most dominant of which involves efficient rewiring of the human fibroblast epigenome to a cardiac fate. Transcription factors are tremendously powerful in exacting these cell fate changes, yet the inventors know from developmental studies that their effects are often intricately interdependent on chromatin remodeling machinery (Wamstad et al., 2012; Sun et al., 2018; Han et al., 2011). In spite of this, relatively little is known regarding the potential for chromatin remodelers in direct cardiac reprogramming. These studies highlight the tremendous therapeutic potential of PHF7 through its ability to rapidly and persistently achieve cardiac cell fate conversion in adult and human fibroblasts in the context of fewer factors, and the ability of PHF7 on its own to activate critical TFs and improve cardiac function post-MI. The inventors believe that identification and definition of epigenetic factors, such as PHF7, merits further investigation to not only shed light on the mechanistic basis of cardiac reprogramming, but to also more rapidly propel these technologies past the epigenomic barriers of human cells to the clinic.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods, and in the steps or in the sequence of steps of the methods described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
“Antibodies: A Laboratory Manual,” Cold Spring Harbor Press, Cold Spring Harbor, NY, 1988.
Angel et al., Cell, 49:729, 1987a.
Angel et al., Cell, 49:729, 1987b.
Baichwal and Sugden, In: Gene Transfer, Kucherlapati (Ed), NY, Plenum Press, 117-148, 1986.
Banerji et al., Cell, 27(2 Pt 1):299-308, 1981.
Banerji et al., Cell, 33(3):729-740, 1983.
Barnes et al., J. Biol. Chem., 272(17):11510-7, 1997.
Basson et al., Nat. Genet., 15(1):30-35, 1997.
Benvenisty and Neshif, Proc. Natl. Acad. Sci. USA, 83:9551-9555, 1986.
Berkhout et al., Cell, 59:273-282, 1989.
Bhavsar et al., Genomics, 35(1):11-23, 1996.
Blanar et al., EMBO J., 8:1139, 1989.
Bodine and Ley, EMBO J., 6:2997, 1987.
Boshart et al., Cell, 41:521, 1985.
Bosze et al., EMBO J., 5(7):1615-1623, 1986.
Braddock et al., Cell, 58:269, 1989.
Branon et al., Nat. Biotechnol. 36:880-887, 2018.
Bulla and Siddiqui, J. Virol., 62:1437, 1986.
Campbell and Villarreal, Mol. Cell. Biol., 8:1993, 1988.
Campere and Tilghman, Genes and Dev., 3:537, 1989.
Campo et al., Nature, 303:77, 1983.
Capaldi et al., Biochem. Biophys. Res. Comm., 74(2):425-433, 1977.
Celander and Haseltine, J. Virology, 61:269, 1987.
Celander et al., J. Virology, 62:1314, 1988.
Chandler et al., Cell, 33:489, 1983.
Chatterjee et al., Proc. Natl. Acad. Sci. USA, 86:9114, 1989.
Chen and Okayama, Mol. Cell Biol., 7(8):2745-2752, 1987.
Christoforou et al., PLoS One, 8, 2013.
Choi et al., Cell, 53:519, 1988.
Chronis et al., Cell, 168:442-459 e420, 2017.
Coffin, In: Virology, Fields et al. (Eds.), Raven Press, NY, 1437-1500, 1990.
Cohen et al., J. Cell. Physiol., 5:75, 1987.
Corces et al., Nat. Methods 14:959-962, 2017.
Costa et al., Mol. Cell. Biol., 8:81-90, 1988.
Coupar et al., Gene, 68:1-10, 1988.
Cripe et al., EMBO J., 6:3745, 1987.
Culotta and Hamer, Mol. Cell. Biol., 9:1376-1380, 1989.
Dandolo et al., J. Virology, 47:55-64, 1983.
De Villiers et al., Nature, 312(5991):242-246, 1984.
Devine et al., Elife 3, 2014.
Deschamps et al., Science, 230:1174-1177, 1985.
Dubensky et al., Proc. Natl. Acad. Sci. USA, 81:7529-7533, 1984.
Edbrooke et al., Mol. Cell. Biol., 9:1908-1916, 1989.
Edlund et al., Science, 230:912-916, 1985.
Fechheimer et al., Proc Natl. Acad. Sci. USA, 84:8463-8467, 1987.
Feng and Holland, Nature, 334:6178, 1988.
Ferkol et al., FASEB J., 7:1081-1091, 1993.
Fischer, Med. Res. Rev., 27(6):755-796, 2007.
Foecking and Hofstetter, Gene, 45(1):101-105, 1986.
Fraley et al., Proc. Natl. Acad. Sci. USA, 76:3348-3352, 1979.
Franz et al., Cardoscience, 5(4):235-43, 1994.
Friedmann, Science, 244:1275-1281, 1989.
Fujita et al., Cell, 49:357, 1987.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gilles et al., Cell, 33:717, 1983.
Gloss et al., EMBO J., 6:3735, 1987.
Godbout et al., Mol. Cell. Biol., 8:1169, 1988.
Goodbourn and Maniatis, Proc. Natl. Acad. Sci. USA, 85:1447, 1988.
Goodbourn et al., Cell, 45:601, 1986.
Gopal, Mol. Cell Biol., 5:1188-1190, 1985.
Gopal-Srivastava et al., J. Mol. Cell. Biol., 15(12):7081-90, 1995.
Graham and van der Eb, Virology, 52:456-467, 1973.
Greene et al., Immunology Today, 10:272, 1989
Grosschedl and Baltimore, Cell, 41:885, 1985.
Grunhaus and Horwitz, Seminar in Virology, 3:237-252, 1992.
Han et al., Circ Res, 108:378-396, 2011.
Hang et al., Nature, 466:62-67, 2010.
Harland and Weintraub, J. Cell Biol., 101(3):1094-1099, 1985.
Hashimoto et al., Cell stem cell 25:69-86 e65, 2019.
Haslinger and Karin, Proc. Natl. Acad. Sci. USA, 82:8572, 1985.
Hauber and Cullen, J. Virology, 62:673, 1988.
He et al., Proc Natl Acad Sci USA, 108:5632-5637, 2011.
Hen et al., Nature, 321:249, 1986.
Hensel et al., Lymphokine Res., 8:347, 1989.
Herr and Clarke, Cell, 45:461, 1986.
Hersdorffer et al., DNA Cell Biol., 9:713-723, 1990.
Hirochika et al., J. Virol., 61:2599, 1987.
Hitraya et al., Arthritis Rheum. 41(11):2048-58 1998.
Holbrook et al., Virology, 157:211, 1987.
Horlick and Benfield, Mol. Cell. Biol., 9:2396, 1989.
Horwich et al. J. Virol., 64:642-650, 1990.
Hota et al., Development 146, 2019.
Huang et al., Cell, 27:245, 1981.
Hug et al., Mol. Cell. Biol., 8:3065-3079, 1988.
Hwang et al., Mol. Cell. Biol., 10:585, 1990.
Ieda et al., Cell, 142:375-386, 2010.
Ifkovits, PLoS One 9, e89678, 2014.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Joseph et al., Cancer Cell 13(2):129-40, 2008.
Johnson et al., Mol. Cell. Biol., 9(8):3393-3399, 1989.
Kadesch and Berg, Mol. Cell. Biol., 6:2593, 1986.
Kaneda et al., Science, 243:375-378, 1989.
Karin et al., Mol. Cell. Biol., 7:606, 1987.
Katinka et al., Cell, 20:393, 1980.
Kato et al., J Biol Chem., 266(6):3361-3364, 1991.
Kelly et al., J. Cell Biol., 129(2):383-96, 1995.
Kiledjian et al., Mol. Cell. Biol., 8:145, 1988.
Kimura et al., Dev. Growth Differ., 39(3):257-65, 1997.
Klamut et al., Mol. Cell. Biol., 10:193, 1990.
Klein et al., Nature, 327:70-73, 1987.
Koch et al., Mol. Cell. Biol., 9:303, 1989.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984a.
Kriegler et al., Cell, 53:45, 1988.
Kriegler et al., In: Cancer Cells 2/Oncogenes and Viral Genes, Van de Woude et al. eds, Cold Spring Harbor, Cold Spring Harbor Laboratory, 1984b.
Kuhl et al., Cell, 50:1057, 1987.
Kunz et al., Nucl. Acids Res., 17:1121, 1989.
LaPointe et al., J. Biol. Chem., 263(19):9075-8, 1988.
Larsen et al., Proc. Natl. Acad. Sci. USA., 83:8283, 1986.
Laspia et al., Cell, 59:283, 1989.
Latimer et al., Mol. Cell. Biol., 10:760, 1990.
Laughlin et al., J. Virol., 60(2):515-524, 1986.
Lebkowski et al., Mol. Cell. Biol., 8(10):3988-3996, 1988.
Lee et al., Nature, 294:228, 1981.
Lescroart et al., Science, 359:1177-1181, 2018.
Levinson et al., Nature, 295:79, 1982.
Lickert et al., Nature, 432:107-112, 2004.
Lin et al., Mol. Cell. Biol., 10:850, 1990.
Liu et al., Stem Cell Res, 16:507-518, 2016.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Macejak and Sarnow, Nature, 353:90-94, 1991.
Majors and Varmus, Proc. Natl. Acad. Sci. USA, 80:5866, 1983.
Markowitz et al., J. Virol., 62:1120-1124, 1988.
McDermott et al., Mol. Cell. Biol., 13(4):2564-2577, 1993.
McLaughlin et al., J. Virol., 62(6):1963-1973, 1988.
McNeall et al., Gene, 76:81, 1989.
Miksicek et al., Cell, 46:203, 1986.
Molkentin et al., Mol. Cell. Biol., 16(6):2627-2636, 1996.
Mordacq and Linzer, Genes and Dev., 3:760, 1989.
Moreau et al., Nucl. Acids Res., 9:6047, 1981.
Moss et al., J. Gen. Physiol., 108(6):473-84, 1996.
Muesing et al., Cell, 48:691, 1987.
Muzyczka, Curr. Topics Microbiol. Immunol., 158:97-129, 1992.
Naldini et al., Science, 272(5259):263-267, 1996.
Nam et al., Development, 141:4267-4278, 2014.
Ng et al., Nuc. Acids Res., 17:601, 1989.
Nicolas and Rubinstein, Stoneham: Butterworth, pp. 494-513, 1988.
Nicolau and Sene, Biochim. Biophys. Acta, 721:185-190, 1982.
Nicolau et al., Methods Enzymol., 149:157-176, 1987.
Okada et al., American Journal of Physiology: Renal Physiology, 275 (2): F306-F314, 1998.
Ondek et al., EMBO J., 6:1017, 1987.
Ornitz et al., Mol. Cell. Biol., 7:3466, 1987.
Palmiter et al., Cell, 29:701, 1982.
Pech et al., Mol. Cell. Biol., 9:396, 1989.
Pelletier and Sonenberg, Nature, 334:320-325, 1988.
Perales et al., Proc. Natl. Acad. Sci. USA, 91(9):4086-4090, 1994.
Perez-Stable and Constantini, Mol. Cell. Biol., 10:1116, 1990.
Physicians Desk Reference.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Potter et al., Proc. Natl. Acad. Sci. USA, 81:7161-7165, 1984.
Queen and Baltimore, Cell, 35:741, 1983.
Quinn et al., Mol. Cell. Biol., 9:4713, 1989.
Reisman and Rotter, Mol. Cell. Biol., 9:3571, 1989.
Remington's Pharmaceutical Sciences, 15^{th} ed., 1035-1038 and 1570-1580, Mack Publishing Company, PA, 1980.
Resendez Jr. et al., Mol. Cell. Biol., 8:4579, 1988.
Ridgeway, In: Vectors: A Survey of Molecular Cloning Vectors and Their Uses, Rodriguez et al. (Eds.), Stoneham: Butterworth, 467-492, 1988.
Ripe et al., Mol. Cell. Biol., 9:2224, 1989.
Rippe et al., Mol. Cell Biol., 10:689-695, 1990.
Rittling et al., Nuc. Acids Res., 17:1619, 1989.
Rosen et al., Cell, 41:813, 1988.
Sakai et al., Genes and Dev., 2:1144, 1988.
Saint-Andre et al., Genome Res, 26:385-396, 2016.
Satake et al., J. Virology, 62:970, 1988.
Schaffner et al., J. Mol. Biol., 201:81, 1988.
Searle et al., Mol. Cell. Biol., 5:1480, 1985.
Sharp and Marciniak, Cell, 59:229, 1989.
Shaul and Ben-Levy, EMBO J., 6:1913, 1987.
Sherman et al., Mol. Cell. Biol., 9:50, 1989.
Sleigh and Lockett, J. EMBO, 4:3831, 1985.
Song et al., Nature, 485:599-604, 2012.
Spalholz et al., Cell, 42:183, 1985.
Spandau and Lee, J. Virology, 62:427, 1988.
Spandidos and Wilkie, EMBO J., 2:1193, 1983.
Stanton et al., Nat Genet, 49:282-288, 2017.
Stephens and Hentschel, Biochem. J., 248:1, 1987.
Stone et al., Cell stem cell, 25:87-102 e109, 2019.
Stuart et al., Nature, 317:828, 1985.
Sullivan and Peterlin, Mol. Cell. Biol., 7:3315, 1987.
Sun et al., Biol Open 7, 2018.
Swartzendruber and Lehman, J. Cell. Physiology, 85:179, 1975.
Takahashi & Yamanaka, Cell, 126:663-676, 2006.
Takebe et al., Mol. Cell. Biol., 8:466, 1988.
Takeuchi & Bruneau, Nature, 459:708-711, 2009.
Takeuchi et al., Proc Natl Acad Sci USA, 104:846-851, 2007.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Temin, In: Gene Transfer, Kucherlapati (Ed.), NY, Plenum Press, 149-188, 1986.
Terrett et al., Nat. Genet., 6(4):401-404, 1994.
The Merck Index, 11th Edition.
Thiesen et al., J. Virology, 62:614, 1988.
Tratschin et al., Mol. Cell. Biol., 4:2072-2081, 1984.
Tronche et al., Mol. Biol. Med., 7:173, 1990.
Tronche et al., Mol. Cell. Biol., 9:4759, 1989.
Trudel and Constantini, Genes and Dev., 6:954, 1987.
Tur-Kaspa et al., Mol. Cell Biol., 6:716-718, 1986.
U.S. Pat. No. 5,139,941
U.S. Pat. No. 5,580,859
U.S. Pat. No. 5,670,488
U.S. Pat. No. 5,672,681
U.S. Pat. No. 5,994,136
U.S. Pat. No. 6,013,516
Vannice and Levinson, J. Virology, 62:1305, 1988.
Varmus et al., Cell, 25:23-36, 1981.
Vasseur et al., Proc Natl. Acad. Sci. U.S.A., 77:1068, 1980.
Wagner et al., Proc. Natl. Acad. Sci. USA 87(9):3410-3414, 1990.
Wamstad et al., Cell, 151:206-220, 2012.
Wang et al., Genomics, 103:222-228, 2014.
Wang et al., Development, 146:2019.
Wang and Calame, Cell, 47:241, 1986.
Weber et al., Cell, 36:983, 1984.
Weinberger et al. Mol. Cell. Biol., 8:988, 1984.
Wong et al., Gene, 10:87-94, 1980.
Wu and Wu, Adv. Drug Delivery Rev., 12:159-167, 1993.
Wu and Wu, Biochemistry, 27: 887-892, 1988.
Wu and Wu, J. Biol. Chem., 262:4429-4432, 1987.
Whyte et al., Cell, 153:307-319, 2013.
Yamauchi-Takihara et al., Proc. Natl. Acad. Sci. USA, 86(10):3504-3508, 1989.
Yang et al., Proc. Natl. Acad. Sci. USA, 87:9568-9572, 1990.
Yang et al., Dev Cell, 22:1041-1051, 2012.
Yang et al., Genetics, 206:1939-1949, 2017.
Young et al., In: Handbook of Applied Therapeutics, 7.1-7.12 and 9.1-9.10, 1989.
Yutzey et al. Mol. Cell. Biol., 9:1397, 1989.
Zelenin et al., FEBS Lett., 280:94-96, 1991.
Zhou et al., Proc Natl Acad Sci USA 112:11864-11869, 2015.
Zhou et al., Cell stem cell, 18:382-395, 2016.
Zhou et al., Genes & development, 31:1770-1783, 2017.
Zhou et al., Stem Cells Int, 2018:3814747, 2018.
Zlatic et al., J. Vis. Exp. 1855, 2010.
Ziober and Kramer, J. Bio. Chem., 271(37):22915-22922, 1996.
Zufferey et al., Nat. Biotechnol., 15(9):871-875, 1997.

1. A method of reprogramming a cardiac fibroblast comprising contacting said cardiac fibroblast with PHF7.

2. The method of claim 1, further comprising contacting said cardiac fibroblast with TBX5.

3. The method of claim 1, further comprising contacting said cardiac fibroblast with MEF2C.

4. The method of claim 1, further comprising contacting said cardiac fibroblast with GATA4.

5. The method of claim 1, further comprising contacting said cardiac fibroblast with HAND2.

6. The method of claim 1, further comprising contacting said cardiac fibroblast with MYCD and/or phosphorylated AKT1.

7. The method of claim 1, wherein contacting comprises delivering PHF7 proteins to said cardiac fibroblast.

8. The method of claim 7, further comprising delivering TBX5 and/or MEF2C proteins to said cardiac fibroblast.

9. The method of claim 7, further comprising delivering PHF7 with GATA4, MEF2C, and TBX5 proteins to said cardiac fibroblast.

10. The method of claim 7, further comprising delivering HAND2 proteins to said cardiac fibroblast.

11. The method of claim 7, further comprising delivering said MYCD proteins to said cardiac fibroblast.

12. The method of claim 7, further comprising delivering phosphorylated AKT1 proteins to said cardiac fibroblast.

13. The method of claim 1, wherein PHF7 proteins comprise a heterologous cell permeability peptide (CPP).

14. The method of claim 2, wherein one, two or all three of PHF7, TBX5 and/or MEF2C proteins comprise a heterologous cell permeability peptide (CPP).

15. The method of claim 2, wherein PHF7 with one, two, three, four, five or all six of MYCD, GATA4, HAND2, MEF2C, TBX5, and phosphorylated AKT1 comprise a heterologous cell permeability peptide (CPP).

16. The method of claim 1, wherein contacting comprises delivering PHF7 expression cassettes to said cardiac fibroblast.

17. The method of claim 16, further comprising delivering an expression cassette encoding TBX5 to said cardiac fibroblast.

18. The method of claim 16, further comprising delivering PHF7 expression cassettes with one more expression cassettes encoding MYCD, GATA4, HAND2, MEF2C, TBX5, and/or phosphorylated AKT1 to said cardiac fibroblast.

19. The method of claim 17, wherein a MEF2C encoding nucleic acid segment is in the same expression cassette as either or both of PHF7 and TBX5.

20. The method of claim 18, wherein PHF7, MYCD, GATA4, HAND2, MEF2C, TBX5, and/or phosphorylated AKT1 encoding nucleic acid segments are in the same expression cassette.

21. The method of claim 17, wherein a MEF2C encoding nucleic acid segment is in a distinct expression cassette from either or both of PHF7 and TBX5.

22. The method of claim 21, wherein PHF7, MYCD, GATA4, HAND2, MEF2C, TBX5, and/or phosphorylated AKT1 encoding nucleic acid segments are in distinct expression cassettes.

23. The method of claim 16, wherein said expression cassette or cassettes are comprised in one or more replicable vectors.

24. The method of claim 23, wherein said one or more replicable vectors are one or more viral vectors.

25. The method of claim 24, wherein said one or more viral vectors are one or more adeno-associated virus (AAV), non-integrated lentivirus, adenoviral vectors or retroviral vectors.

26. The method of claim 23, wherein said one or more replicable vectors are non-viral vectors.

27. The method of claim 23, wherein said one or more replicable vectors are modified RNA delivery vectors.

28. The method of claim 26, wherein said one or more non-viral vectors are disposed in a lipid delivery vehicle.

29. The method of claim 1, further comprising contacting said cardiac fibroblast with an anti-inflammatory agent, small molecule, or micro-RNA.

30. A method of treating a subject having suffered a myocardial infarct (MI) comprising delivering to said subject PHF7.

31. The method of claim 30, further comprising delivering to said subject TBX5.

32. The method of claim 30, further comprising delivering to said subject MEF2C.

33. The method of claim 39, further comprising delivering to said subject GATA4.

34. The method of claim 30, further comprising delivering to said subject HAND2.

35. The method of claim 30, further comprising delivering to said subject MYCD and/or phosphorylated AKT1.

36. The method of claim 30, wherein delivering comprises administration of PHF7 proteins.

37. The method of claim 30, wherein delivering further comprises administration of TBX5 proteins.

38. The method of claim 30, wherein delivering further comprises administration of MEF2C proteins.

39. The method of claim 30, wherein delivering comprises administration of PHF7 proteins with one, two, three, four, five or all six of MCYD, phosphorylated AKT1, GATA4, HAND2, MEF2C, and/or TBX5 proteins.

40. The method of claim 36, wherein PHF7 and one, two, three, or all six of MCYD, phosphorylated AKT1, GATA4, HAND2, MEF2C, and/or TBX5 comprise a heterologous cell permeability peptide (CPP).

41. The method of claim 30, wherein delivering comprises administering PHF7 expression cassettes to said cardiac fibroblast, such as by intracardiac or intracoronary injection.

42. The method of claim 41, further comprising administering one or more expression cassettes encoding TBX5 and/or MEF2C to said cardiac fibroblast, such as by intracardiac or intracoronary injection.

43. The method of claim 41, further comprising administering an expression cassette encoding PHF7 with a combination of expression cassettes encoding GATA4, HAND2, MEF2C, TBX5, MYCD, and/or phosphorylated AKT1, such as by intracardiac or intracoronary injection.

44. The method of claim 42, wherein a PHF7 encoding nucleic acid segment is in the same expression cassette as either or both of MEF2C and TBX5.

45. The method of claim 42, wherein a PHF7 encoding nucleic acid segment is in the same expression cassette as either or both of GATA4, HAND2, MEF2C, TBX5, MYCD, and/or phosphorylated AKT1.

46. The method of claim 42, wherein a MEF2C encoding nucleic acid segment is in a distinct expression cassette from either or both of PHF7 and TBX5.

47. The method of claim 42, wherein a PHF7 encoding nucleic acid segment is in a distinct expression cassette from either or all of GATA4, HAND2, MEF2C, TBX5, MYCD, and/or phosphorylated AKT1.

48. The method of claim 41, wherein said expression cassettes are comprised in one or more replicable vectors.

49. The method of claim 48, wherein said one or more replicable vectors are one or more viral vectors.

50. The method of claim 49, wherein said one or more viral vectors are one or more adeno-associated virus (AAV), non-integrated lentivirus, adenoviral vectors or retroviral vectors.

51. The method of claim 48, wherein said one or more replicable vectors are non-viral vectors and/or modified RNA delivery vectors.

52. The method of claim 51, wherein said one or more non-viral vectors are disposed in a lipid delivery vehicle.

53. The method of claim 30, further comprising administering to said subject an anti-inflammatory agent, small molecule, micro-RNA, oxygen, aspirin, nitroglycerin, a fibrinolytic, percutaneous coronary intervention, and/or surgical correction through coronary bypass.

54. The method of claim 30, wherein one, two, three, four, five, six or all seven of PHF7, TBX5, MEF2C, GATA4, HAND2, phosphorylated AKT1, and/or MYCD proteins or expression cassettes are delivered 24 hours to one month following said MI.

55. The method of claim 36, wherein at least one of said PHF7, TBX5, and MEF2C proteins are delivered multiple times, such as 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 times, or daily.

56. The method of claim 36, wherein PHF7, TBX5, and optionally MEF2C, GATA4, HAND2, phosphorylated AKT1, and/or MYCD proteins are delivered multiple times, such as 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 times, or daily.

57. The method of claim 36, wherein at least one of said PHF7, TBX5, and optionally MEF2C, expression cassettes are delivered multiple times, such as 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 times, or daily.

58. The method of claim 36, wherein PHF7, TBX5, and optionally MEF2C, GATA4, HAND2, phosphorylated AKT1, and/or MYCD expression cassettes are delivered multiple times, such as 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 times, or daily.

59. The method of claim 30, wherein said MI is non-ST-elevated MI.

60. The method of claim 39, wherein said MI is ST-elevated MI.

61. A method of reversing, preventing, or delaying development or worsening of cardiac hypertrophy or heart failure in a subject having suffered a myocardial infarct (MI) comprising providing to said subject PHF7, and optionally further providing TBX5, MEF2C, GATA4, HAND2, phosphorylated AKT1, and/or MYCD as proteins or as expression cassettes coding such proteins.

62. The method of claim 61, further comprising administering to said subject a secondary anti-hypertrophic or heart failure therapy.

63. The method of claim 62, wherein the secondary therapy comprises combinations of a PKD inhibitor, a beta blocker, an ionotrope, a diuretic, ACE-I, AII antagonist, BNP, a Ca⁺⁺-blocker, an SGLT2 inhibitor, a GLP-1 agonist, a neprilysin inhibitor, or an HDAC inhibitor.

64. The method of claim 61, wherein the method may result in preventing, reversing delaying development or worsening of cardiac hypertrophy.

65. The method of claim 61, wherein the method may result in reversing, preventing, delaying development or worsening one or more of decreased exercise capacity, diastolic dysfunction, decreased cardiac ejection fraction, increased left ventricular end diastolic pressure, increased pulmonary capillary wedge pressure, decreased cardiac output or cardiac index, increased pulmonary artery pressures, increased left ventricular end systolic and diastolic dimensions, increased left and right ventricular wall stress, increased wall tension, decreased quality of life, and/or increased disease related morbidity or mortality, and wherein reversing comprises improvement and/or increase in exercise capacity, increased cardiac ejection fraction, decreased left ventricular end diastolic pressure, decreased pulmonary capillary wedge pressure, increased cardiac output or cardiac index, decreased pulmonary artery pressures, decreased left ventricular end systolic and/or diastolic dimensions, decreased left and right ventricular wall stress, decreased wall tension, increased quality of life, and/or increased disease related morbidity or mortality as compared to measures prior to PHF7 and/or PHF7, TBX5, optionally MEF2C, GATA4, HAND2, phosphorylated AKT1, and/or MYCD cocktail delivery.

66. The method of claim 61, wherein PHF7 proteins, and optionally TBX5, MEF2C, GATA4, HAND2, phosphorylated AKT1, and/or MYCD proteins are administered to said subject.

67. The method of claim 61, wherein PHF7 expression cassettes, and optionally TBX5, MEF2C, GATA4, HAND2, phosphorylated AKT1, and/or MYCD expression cassettes are administered to said subject.

68. The method of claim 61, further comprising administering an anti-inflammatory agent, small molecule, or micro-RNA to said subject.

69. The method of claim 61, wherein PHF7, and optionally MEF2C, GATA4, HAND2, phosphorylated AKT1, and/or MYCD proteins or expression cassettes are delivered multiple times, such as 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 times, or daily.

70. A method of improving exercise tolerance and/or reducing a decrease in exercise tolerance of a subject having suffered a myocardial infarction comprising delivering to said subject PHF7, and optionally TBX5, MEF2C, GATA4, HAND2, phosphorylated AKT1, and/or MYCD proteins or expression cassettes coding therefor.

71. A method of reducing incidence of hospitalization or length of stay of a subject having suffered a myocardial infarction and/or heart failure comprising delivering to said subject PHF7, and optionally TBX5, MEF2C, GATA4, HAND2, phosphorylated AKT1, and/or MYCD proteins or expression cassettes coding therefor.

72. A method of improving cardiac function, preventing a decrease is cardiac function and/or quality of life of a subject, such as one having suffered a myocardial infarction, comprising delivering to said subject PHF7, and optionally TBX5, MEF2C, GATA4, HAND2, phosphorylated AKT1, and/or MYCD proteins or expression cassettes coding therefor.

73. A method of decreasing morbidity of a subject having suffered a myocardial infarction comprising delivering to said subject PHF7, and optionally TBX5, MEF2C, GATA4, HAND2, phosphorylated AKT1, and/or MYCD proteins or expression cassettes coding therefor.

74. A method of decreasing mortality of a subject having suffered a myocardial infarction comprising delivering to said subject PHF7, and optionally TBX5, MEF2C, GATA4, HAND2, phosphorylated AKT1, and/or MYCD proteins or expression cassettes coding therefor.

75. The method of claim 70, further comprising administering and anti-inflammatory agent, small molecule, or micro-RNA to said subject.

76. A method of activating GATA4 and/or HAND2 in a cardiac cell comprising delivering to said cell PHF7, either by protein delivery or by delivery of an expression construct encoding PHF7.


SEQUENCE LISTING (14 sequences)

SEQ ID NO: 1 - RNA, 2127 nt, Homo sapiens
  source 1..2127: mol_type = mRNA
agtcttcgccggcgcagtcaccgcgcagggcggccgcccggaggtagctaccacggcctgtgtcaacgactaaagctccagtacagcggcgccctcagacagctgggagggtggctctggccgggagcggcggccggtgagctaccgcgaggaggagcggcggaggcgacctcggcccggccctgcactggccgcccggcaggcgcgacatgagcctggtctggcatccgcgggatgctccttaagccccttctccggctgttaacctccggggaacggttgtgaccacaccgacacgtattttacagataaatcattcttgcggcggcgggtcgaacacgtttatttattttttattttctcaacaagcttttacccagcacctgtccagtgaaacaacttgataatcgtttcgaggggcgtccgccgggttaggaagccactgcctggcagcttgtggaagcctcatttgcaaagccacccctcagatgttttgaagatcgtgacgtcttgtaactagcagtgtgtgcacagaatcctactcaaggaacgtcttggcccagcgatgcaaagaactgaagtttcaagctggaagagcctgtattgtcctcacaatagtatagaagaattcaagagaggagagagagacagcaccgaatgaagactgtaaaagaaaagaaggaatgccagagattgagaaaatctgccaagactaggagggtaacccagaggaaaccgtcttcagggcctgtttgctggctatgccttcgagaacctggggatcccgaaaaattaggggaatttcttcagaaagacaatatcagcgtgcattatttctgtcttatcttatctagtaagctgcctcagaggggccagtccaacagaggcttccatggatttctgcctgaagacatcaaaaaggaggcagcccgggcttctaggaagatctgctttgtgtgcaagaaaaagggagctgctatcaactgccagaaggatcagtgcctcagaaacttccatctgccttgtggccaagaaaggggttgcctttcacaattttttggagagtacaaatcattttgtgacaaacatcgcccaacacagaacatccaacatgggcatgtgggggaggaaagctgcatcttatgttgtgaagacttatcccaacagagtgttgagaacatccagagcccgtgttgtagtcaagccatctaccaccgcaagtgcatacagaaatatgcccacacatcagcaaagcatttcttcaaatgtccacagtgtaacaatcgaaaagagtttcctcaagaaatgctgagaatgggaattcatattccagacagagatgctgcctgggaactcgagccaggggctttctcagacttatatcagcgctatcagcactgtgatgcccccatctgtctgtatgaacaaggcagagacagctttgaggatgaagggaggtggtgcctcattctgtgtgctacatgcggatcccacggaacccacagggactgctcctctcttagatctaacagtaagaaatgggagtgtgaggagtgttcacctgctgcagccacagactacatacctgaaaactcaggggacatcccttgctgcagcagcaccttccaccctgaggaacatttctgcagagacaacaccttggaagagaatccgggcctttcttggactgattggccagaaccttccttattagaaaagccagagtcctctcgtggcaggaggagctactcctggaggtccaagggtgtcagaatcactaacagctgcaaaaaatccaagtaacaccttctgagtagctgctgtcccacacaatagggtatgaagctgcgctcctccatcgggtttggggagggagcactctgggactgtgagacaaggaagcagggccagcagtgagactatgagccaagcaaagagaagtctcagtggagcatgaggagggagcagtccagatgccaacaaggaaatgcgtttatggctacaagagtgcctctgctttctcctcctctcctcccaccaaggattcttccaccttaatcttgttttcatatgcctcttcttacttcacccatgtttgttgttatgcaaataaaggttttctctcca

SEQ ID NO: 2 - protein, 381 aa, Homo sapiens
MKTVKEKKECQRLRKSAKTRRVTQRKPSSGPVCWLCLREPGDPEKLGEFLQKDNISVHYFCLILSSKLPQRGQSNRGFHGFLPEDIKKEAARASRKICFVCKKKGAAINCQKDQCLRNFHLPCGQERGCLSQFFGEYKSFCDKHRPTQNIQHGHVGEESCILCCEDLSQQSVENIQSPCCSQAIYHRKCIQKYAHTSAKHFFKCPQCNNRKEFPQEMLRMGIHIPDRDAAWELEPGAFSDLYQRYQHCDAPICLYEQGRDSFEDEGRWCLILCATCGSHGTHRDCSSLRSNSKKWECEECSPAAATDYIPENSGDIPCCSSTFHPEEHFCRDNTLEENPGLSWTDWPEPSLLEKPESSRGRRSYSWRSKGVRITNSCKKSK

SEQ ID NO: 3 - RNA, 3921 nt, Homo sapiens
  source 1..3921: mol_type = mRNA
catgccttatgcaagagacctcagtcccccggaacaactcgatttccttccaatagaggtctgaggtggactcccacctcccttcgtgaagagttccctcctctcccccttcctaagaaagtcgatcttggctctatttgtgtcttatgttcatcaccctcattcctccggagaaagccgggttggtttatgtctttatttattcccggggccaagacgtccggaacctgtggctgcgcagacccggcactgataggcgaagacggagagaaatttacctcccgccgctgccccccagccaaacgtgacagcgcgcgggccggttgcgtgactcgtgacgtctccaagtcctataggtgcagcggctggtgagatagtcgctatcgcctggttgcctctttattttactggggtatgcctggtaataaacagtaatatttaatttgtcggagaccacaaaccaaccttgagctgggaggtacgtgctcttcttgacagacgttggaagaagacctggcctaaagaggtctcttttggtggtccttttcaaagtcttcacctgagccctgctctccagcgaggcgcactcctggcttttgcgctccaaagaagaggtgggatagttggagagcagaaccttgcgcgggcacagggccctgggcgcaccatggccgacgcagacgagggctttggcctggcgcacacgcctctggagcctgacgcaaaagacctgccctgcgattcgaaacccgagagcgcgctcggggcccccagcaagtccccgtcgtccccgcaggccgccttcacccagcagggcatggagggaatcaaagtgtttctccatgaaagagaactgtggctaaaattccacgaagtgggcacggaaatgatcataaccaaggctggaaggcggatgtttcccagttacaaagtgaaggtgacgggccttaatcccaaaacgaagtacattcttctcatggacattgtacctgccgacgatcacagatacaaattcgcagataataaatggtctgtgacgggcaaagctgagcccgccatgcctggccgcctgtacgtgcacccagactcccccgccaccggggcgcattggatgaggcagctcgtctccttccagaaactcaagctcaccaacaaccacctggacccatttgggcatattattctaaattccatgcacaaataccagcctagattacacatcgtgaaagcggatgaaaataatggatttggctcaaaaaatacagcgttctgcactcacgtctttcctgagactgcgtttatagcagtgacttcctaccagaaccacaagatcacgcaattaaagattgagaataatccctttgccaaaggatttcggggcagtgatgacatggagctgcacagaatgtcaagaatgcaaagtaaagaatatcccgtggtccccaggagcaccgtgaggcaaaaagtggcctccaaccacagtcctttcagcagcgagtctcgagctctctccacctcatccaatttggggtcccaataccagtgtgagaatggtgtttccggcccctcccaggacctcctgcctccacccaacccatacccactgccccaggagcatagccaaatttaccattgtaccaagaggaaagaggaagaatgttccaccacagaccatccctataagaagccctacatggagacatcacccagtgaagaagattccttctaccgctctagctatccacagcagcagggcctgggtgcctcctacaggacagagtcggcacagcggcaagcttgcatgtatgccagctctgcgccccccagcgagcctgtgcccagcctagaggacatcagctgcaacacgtggccaagcatgccttcctacagcagctgcaccgtcaccaccgtgcagcccatggacaggctaccctaccagcacttctccgctcacttcacctcggggcccctggtccctcggctggctggcatggccaaccatggctccccacagctgggagagggaatgttccagcaccagacctccgtggcccaccagcctgtggtcaggcagtgtgggcctcagactggcctgcagtcccctggcacccttcagccccctgagttcctctactctcatggcgtgccaaggactctatcccctcatcagtaccactctgtgcacggagttggcatggtgccagagtggagcgacaatagctaaagtgaggcctgcttcacaacagacatttcctagagaaagagagagagagaggagaaagagagagaaggagagagacagtagccaagagaaccccacggacaagatttttcatttcacccaatgttcacatctgcactcaaggtcgctggatgctgatctaatcagtagcttgaaaccacaattttaaaaatgtgactttcttgttttgtctcaaaacttaaaaaaacaaacacaaaaagatgagtcccaccccccactaccaccacacccatcaaccagccacattcacgctactccccagatctcttcccccattccttcttttgggctctagaaagtcttgcctcattgagtgtttttccctagtgcgtagttggagtctgtccctgtcttggtgttaatgttgacattgttatataataaatgataatatatttttttctttcaattttcttaatgggacccagtcccttatttggggggaggtctgaggcaagtatatttcaaaatatgtacttgcgggattcccttcaagtaaaccatccctgaaacctaaattcacgtttccccttgactaagaaaagcacctacctctgccatgtgatgtttctgaaaagcctctgtatgtccccatttgctttggttttgtcctgccttctccaatatcacgtgctcagttttgcctctacttacccatggagtcaggataacactgacgctccctggcatcctatcttattcagccctaccatcttgccagctctgtctttccagctgtctgtcgctaaaacgtggcctatagcttcccttccggaaagcttgctttgaaaaacttaaaaagcccccgtttacatgtaggcaggactgtgataacagtgcaagctctgtgttgacaagagttgtggacaaaaagccaaaataaatattcttcctgattaaaaaaattttttttgaaaaaaacaaggccagccccaaccttccaaacctccatcaccaacaacccaaactggatgtcaagcaaaatgcacaattcctacagaagaggcaagacacagtcaccaatgatatctcgccaaagaaaccacgcccacaccaatgccaacacaaaactgtgtttactgaaagccgaaaacagtattaaaaaaagtgtgtaagtaaagtgttatggtagggttcttcagatgtaatattttactggtactatttatttataaataggaattctaattaagtaataacatgaaatgaaacccagcataggagctggccaagagcttttaattttattgatactcaaaaccaagtttgtgtttttttgtttttttttgtttttttcctctttcgaatgtgctttgctttttttgattaaaaagaattttttttttccttttttataaacagaccctaataaagagaacagggtaagatgtgaggctgagtgtgtttaagtacgtgagagagtgtgagtgtgtttgtaagtgagtgtccctatgcgattatgtctctttacgttgctaaggggggagggtgaggattaagtactcgtgccttatatttgtgtgccaattaatgcctaataaataccatgtgcttaaacaagtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - protein, 518 aa, Homo sapiens
MADADEGFGLAHTPLEPDAKDLPCDSKPESALGAPSKSPSSPQAAFTQQGMEGIKVFLHERELWLKFHEVGTEMIITKAGRRMFPSYKVKVTGLNPKTKYILLMDIVPADDHRYKFADNKWSVTGKAEPAMPGRLYVHPDSPATGAHWMRQLVSFQKLKLTNNHLDPFGHIILNSMHKYQPRLHIVKADENNGFGSKNTAFCTHVFPETAFIAVTSYQNHKITQLKIENNPFAKGFRGSDDMELHRMSRMQSKEYPVVPRSTVRQKVASNHSPFSSESRALSTSSNLGSQYQCENGVSGPSQDLLPPPNPYPLPQEHSQIYHCTKRKEEECSTTDHPYKKPYMETSPSEEDSFYRSSYPQQQGLGASYRTESAQRQACMYASSAPPSEPVPSLEDISCNTWPSMPSYSSCTVTTVQPMDRLPYQHFSAHFTSGPLVPRLAGMANHGSPQLGEGMFQHQTSVAHQPVVRQCGPQTGLQSPGTLQPPEFLYSHGVPRTLSPHQYHSVHGVGMVPEWSDNS

SEQ ID NO: 5 - RNA, 7281 nt, Homo sapiens
  source 1..7281: mol_type = mRNA
gcagtcacagacacttgagcacacgcgtacacccagacatcttcgggctgctattggattgactttgaaggttctgtgtgggtcgccgtggctgcatgtttgaatcaggtggagaagcacttcaacgctggacgaagtaaagattattgttgttattttttttttctctctctctctctcttaagaaaggaaaatatcccaaggactaatctgatcgggtcttccttcatcaggaacgaatgcaggaatttgggaactgagctgtgcaagtgctgaagaaggagatttgtttggaggaaacaggaaagagaaagaaaaggaaggaaaaaatacataatttcagggacgagagagagaagaaaaacggggactatggggagaaaaaagattcagattacgaggattatggatgaacgtaacagacaggtgacatttacaaagaggaaatttgggttgatgaagaaggcttatgagctgagcgtgctgtgtgactgtgagattgcgctgatcatcttcaacagcaccaacaagctgttccagtatgccagcaccgacatggacaaagtgcttctcaagtacacggagtacaacgagccgcatgagagccggacaaactcagacatcgtggagacgttgagaaagaagggccttaatggctgtgacagcccagaccccgatgcggacgattccgtaggtcacagccctgagtctgaggacaagtacaggaaaattaacgaagatattgatctaatgatcagcaggcaaagattgtgtgctgttccacctcccaacttcgagatgccagtctccatcccagtgtccagccacaacagtttggtgtacagcaaccctgtcagctcactgggaaaccccaacctattgccactggctcacccttctctgcagaggaatagtatgtctcctggtgtaacacatcgacctccaagtgcaggtaacacaggtggtctgatgggtggagacctcacgtctggtgcaggcaccagtgcagggaacgggtatggcaatccccgaaactcaccaggtctgctggtctcacctggtaacttgaacaagaatatgcaagcaaaatctcctcccccaatgaatttaggaatgaataaccgtaaaccagatctccgagttcttattccaccaggcagcaagaatacgatgccatcagtgtctgaggatgtcgacctgcttttgaatcaaaggataaataactcccagtcggctcagtcattggctaccccagtggtttccgtagcaactcctactttaccaggacaaggaatgggaggatatccatcagccatttcaacaacatatggtaccgagtactctctgagtagtgcagacctgtcatctctgtctgggtttaacaccgccagcgctcttcaccttggttcagtaactggctggcaacagcaacacctacataacatgccaccatctgccctcagtcagttgggagcttgcactagcactcatttatctcagagttcaaatctctccctgccttctactcaaagcctcaacatcaagtcagaacctgtttctcctcctagagaccgtaccaccaccccttcgagatacccacaacacacgcgccacgaggcggggagatctcctgttgacagcttgagcagctgtagcagttcgtacgacgggagcgaccgagaggatcaccggaacgaattccactcccccattggactcaccagaccttcgccggacgaaagggaaagtccctcagtcaagcgcatgcgactttctgaaggatgggcaacatgatcagattattacttactagtttttttttttttcttgcagtgtgtgtgtgtgctataccttaatggggaaggggggtcgatatgcattatatgtgccgtgtgtggaaaaaaaaaaagtcaggtactctgttttgtaaaagtacttttaaattgcctcagtgatacagtataaagataaacagaaatgctgagataagcttagcacttgagttgtacaacagaacacttgtacaaaatagattttaaggctaacttcttttcactgttgtgctcctttgcaaaatgtatgttacaatagatagtgtcatgttgcaggttcaacgttatttacatgtaaatagacaaaaggaaacatttgccaaaagcggcagatctttactgaaagagagagcagctgttatgcaacatatagaaaaatgtatagatgcttggacagacccggtaatgggtggccattggtaaatgttaggaacacaccaggtcacctgacatcccaagaatgctcacaaacctgcaggcatatcattggcgtatggcactcattaaaaaggatcagagaccattaaaagaggaccatacctattaaaaaaaaatgtggagttggagggctaacatatttaattaaataaataaataaatctgggtctgcatctcttattaaataaaaatataaaaatatgtacattacattttgcttattttcatataaaaggtaagacagagtttgcaaagcatttgtggctttttgtagtttacttaagccaaaatgtgtttttttccccttgatagcttcgctaatattttaaacagtcctgtaaaaaaccaaaaaggactttttgtatagaaagcactaccctaagccatgaagaactccatgctttgctaaccaagataactgttttctctttgtagaagttttgtttttgaaatgtgtatttctaattatataaaatattaagaatcttttaaaaaaatctgtgaaattaacatgcttgtgtatagctttctaatatatataatattatggtaatagcagaagttttgttatcttaatagcgggaggggggtatatttgtgcagttgcacatttgagtaactattttctttctgttttcttttactctgcttacattttataagtttaaggtcagctgtcaaaaggataacctgtggggttagaacatatcacattgcaacaccctaaattgtttttaatacattagcaatctattgggtcaactgacatccattgtatatactagtttctttcatgctatttttattttgttttttgcatttttatcaaatgcagggcccctttctgatctcaccatttcaccatgcatcttggaattcagtaagtgcatatcctaacttgcccatattctaaatcatctggttggttttcagcctagaatttgatacgctttttagaaatatgcccagaatagaaaagctatgttggggcacatgtcctgcaaatatggccctagaaacaagtgatatggaatttacttggtgaataagttataaattcccacagaagaaaaatgtgaaagactgggtgctagacaagaaggaagcaggtaaagggatagttgctttgtcatccgtttttaattattttaactgacccttgacaatcttgtcagcaatataggactgttgaacaatcccggtgtgtcaggacccccaaatgtcacttctgcataaagcatgtatgtcatctattttttcttcaataaagagatttaatagccatttcaagaaatcccataaagaacctctctatgtccctttttttaatttaaaaaaaatgactcttgtctaatattcgtctataagggattaattttcagaccctttaataagtgagtgccataagaaagtcaatatatattgtttaaaagatatttcagtctaggaaagattttccttctcttggaatgtgaagatctgtcgattcatctccaatcatatgcattgacatacacagcaaagaagatataggcagtaatatcaacactgctatatcatgtgtaggacatttcttatccattttttctcttttacttgcatagttgctatgtgtttctcattgtaaaaggctgccgctgggtggcagaagccaagagaccttattaactaggctatatttttcttaacttgatctgaaatccacaattagaccacaatgcacctttggttgtatccataaaggatgctagcctgccttgtactaatgttttatatattaaaaaaaaaaaatctatcaaccatttcatatatatcccactactcaaggtatccatggaacatgaaagaataacatttatgcagaggaaaaacaaaaacatccctgaaaatatacacactcatacacacacacgcacaggggaataaaataagaaaatcattttcctcaccatagacttgatcccatccttacaacccatccttctaacttgatgtgtataaaatatgcaaacatttcacaaatgttctttgtcatttcaaaatactttagtatatcaatatcagtagataccagtgggtgggaaagggtcattacatgaaaatatgaagaaatagccatattagttttttaacctgcaatttgcctcagcaacaaagaaaaagtgaatttttaatgctgaagataaagtaagctaaagtaccagcagaagccttggctatttatagcagttctgacaatagttttataagaacatgaagagaacagaatcacttgaaaatggatgccagtcatctcttgttcccactactgaattcttataaagtggtggcaagatagggaagggataatctgagaatttttaaaagatgatttaatgagaagaagcacaattttgattttgatgagtcactttctgtaaacaatcttggtctatctttacccttataccttatctgtaatttaccatttattgtatttgcaaagctagtatggtttttaatcacagtaaatcctttgtattccagactttagggcagagccctgagggagtattattttacataacccgtcctagagtaacattttaggcaacattcttcattgcaagtaaaagatccataagtggcattttacacggctgcgagtattgttatatctaatcctattttaaaagatttttggtaatatgaagcttgaatactggtaacagtgatgcaatatacgcaagctgcacaacctgtatattgtatgcattgctgcgtggaggctgtttatttcaacctttttaaaaattgtgttttttagtaaaatggcttattttttcccaaaggtggaatttagcattttgtaatgatgaatataaaaatacctgtcatccccagatcatttaaaagttaactaaagtgagaatgaaaaaacaaaattccaagacactttttaaaagaatgtctgccctcacacacttttatggatttgtttttcttacatacccatcttttaacttagagatagcattttttgccctctttattttgttgtttgtttctccagagagtaaacgctttgtagttctttctttaaaaaacattttttttaaagaagaagaagccacttgaaccctcaataaaggctgttgcctaagcatggcatacttcatctgttctcatttgtgccatctgccgtgatgtcgtcacttttatggcgttaatttcctgccactacagatcttttgaagattgctggaatactggtgtctgttagaatgcttcagactacagatgtaattaaaggcttttcttaatatgttttaaccaaagatgtggagcaatccaagccacatatcttctacatcaaatttttccattttggttattttcataatctggtattgcattttgccttccctgttcatacctcaaattgattcatacctcagtttaattcagagaggtcagttaagtgacggattctgttgtggtttgaatgcagtaccagtgttctcttcgagcaaagtagacctgggtcactgtaggcataggacttggattgcttcagatggtttgctgtatcatttttcttctttttcttttcctggggacttgtttccattaaatgagagtaattaaaatcgcttgtaaatgagggcatacaagcatttgcaacaaatattcaaatagaggctcacagcggcataagctggactttgtcgccactagatgacaagatgttataactaagttaaaccacatctgtgtatctcaagggacttaattcagctgtctgtagtgaataaaagtgggaaattttcaaaagtttctcctgctggaaataaggtataatttgtattttgcagacaattcagtaaagttactggctttcttagtgatgcagtgtccgtggtgcatttttttaaattaatgttttgctgtttaaatttattcaattttattgtgttttttaaaaagcagcttatgccacaagcacaaatgagctttctcagttttcaactcaaaatattatatgaaaagatggatatgtgtgtctgtgtgggaggggtgtgaattggatgacggttcttagggtattacagaattcagccagtggtggaagtctaaggttcttattgaatggttcaacttctttatcaaagaagttccatgaagatctttgataacctgtttaaatgtacagaatttttcaaaagccactgtgaaaagcagtaccataagttattttttaactaaaatctgtagaaagtgtttccaaaagagttccttttttcttcttcgaagtaatttctaaaagttgaaaacataggcttataaatctgaataaattagtgaattcatacattttagagactatcaaatatatccattattaacattcaataataatctctaaaggggaatgtccttaaaaagattgatagaaagattaatttaaaaactgaaaaggaaaacaagacaccaccagttagccagctggagaacttggacttttgcattagtagtgatatctgctccaaaaagaatgcagttcgaggaaatgttgcatgtggcttcaaaatagcagtgctttctgctttgttctcttgtattcattttcagcaaacatgtattaagcaattattatatgtaactcatgtggtgattccaggatgaatgaagttgtagccctttctcttaggaaacttataaactaataggagataaaaggcattctcagggaagtctacaaagtgctatgagggagccactttggccatcctggggcagagcagaccagaggctcagagcatggacacttcctcatccacccagcagaagcctttcacttgattttgaaggtgggcaggattgagaaaggcccagaaggggatgagttcaaagctgtttgtgggcatgtgacccctgcagtcacatatggccctagcttgtgtttcgtttagtgttctgctatcattatcctgataatatcctgataataaaattatccttaataatttta

SEQ ID NO: 6 - protein, 473 aa, Homo sapiens
MGRKKIQITRIMDERNRQVTFTKRKFGLMKKAYELSVLCDCEIALIIFNSTNKLFQYASTDMDKVLLKYTEYNEPHESRTNSDIVETLRKKGLNGCDSPDPDADDSVGHSPESEDKYRKINEDIDLMISRQRLCAVPPPNFEMPVSIPVSSHNSLVYSNPVSSLGNPNLLPLAHPSLQRNSMSPGVTHRPPSAGNTGGLMGGDLTSGAGTSAGNGYGNPRNSPGLLVSPGNLNKNMQAKSPPPMNLGMNNRKPDLRVLIPPGSKNTMPSVSEDVDLLLNQRINNSQSAQSLATPVVSVATPTLPGQGMGGYPSAISTTYGTEYSLSSADLSSLSGFNTASALHLGSVTGWQQQHLHNMPPSALSQLGACTSTHLSQSSNLSLPSTQSLNIKSEPVSPPRDRTTTPSRYPQHTRHEAGRSPVDSLSSCSSSYDGSDREDHRNEFHSPIGLTRPSPDERESPSVKRMRLSEGWAT

SEQ ID NO: 7 - RNA, 3419 nt, Homo sapiens
  source 1..3419: mol_type = mRNA
ggggacttggaggcggccggcgcaggggccgcgagaggcttcgtcgccgctgcagctccgggggctcccaggggagcgtgcgcggaacctccaggcccagcaggaccccggctgcggcgaggaggaaggagccagcctagcagcttctgcgcctgtggccgcgggtgtcctggaggcctctcggtgtgacgagtgggggacccgaaggctcgtgcgccacctccaggcctggacgctgccctccgtcttctgcccccaataggtgcgccggaccttcaggccctggggtgaattcagctgctcctacatcagcttccggaaccaccaaaaattcaaattgggattttccggagtaaacaagagcctagagccctttgctcaatgctggatttaatacgtatatatttttaagcgagttggttttttcccctttgatttttgatcttcgcgacagttcctcccacgcatattatcgttgttgccgtcgttttctctccccgcgtggctccttgacctgcgagggagagagaggacaccgaagccgggagctcgcagggaccatgtatcagagcttggccatggccgccaaccacgggccgccccccggtgcctacgaggcgggcggccccggcgccttcatgcacggcgcgggcgccgcgtcctcgccagtctacgtgcccacaccgcgggtgccctcctccgtgctgggcctgtcctacctccagggcggaggcgcgggctctgcgtccggaggcgcctcgggcggcagctccggtggggccgcgtctggtgcggggcccgggacccagcagggcagcccgggatggagccaggcgggagccgacggagccgcttacaccccgccgccggtgtcgccgcgcttctccttcccggggaccaccgggtccctggcggccgccgccgccgctgccgcggcccgggaagctgcggcctacagcagtggcggcggagcggcgggtgcgggcctggcgggccgcgagcagtacgggcgcgccggcttcgcgggctcctactccagcccctacccggcttacatggccgacgtgggcgcgtcctgggccgcagccgccgccgcctccgccggccccttcgacagcccggtcctgcacagcctgcccggccgggccaacccggccgcccgacaccccaatctcgtagatatgtttgacgacttctcagaaggcagagagtgtgtcaactgtggggctatgtccaccccgctctggaggcgagatgggacgggtcactatctgtgcaacgcctgcggcctctaccacaagatgaacggcatcaaccggccgctcatcaagcctcagcgccggctgtccgcctcccgccgagtgggcctctcctgtgccaactgccagaccaccaccaccacgctgtggcgccgcaatgcggagggcgagcctgtgtgcaatgcctgcggcctctacatgaagctccacggggtccccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagcctggtcttggccgacagtcacggggacataatcactgcgtaatcttccctcttccctcctcaaattcctgcacggacctgggacttggaggatagcaaagaaggaggccctgggctcccaggggccggcctcctctgcctggtaatgactccagaacaacaactgggaagaaacttgaagtcgacaatctggttaggggaagcgggtgttggattttctcagatgcctttacacgctgatgggactggagggagcccacccttcagcacgagcacactgcatctctcctgtgagttggagacttctttcccaagatgtccttgtcccctgcgttccccactgtggcctagaccgtgggttttgcattgtgtttctagcaccgaggatctgagaacaagcggagggccgggccctgggacccctgctccagcccgaatgacggcatctgtttgccatgtacctggatgcgacgggcccctggggacaggcccttgccccatccatccgcttgaggcatggcaccgccctgcatccctaataccaaatctgactccaaaattgtggggtgtgacatacaagtgactgaacacttcctggggagctacaggggcacttaacccaccacagcacagcctcatcaaaatgcagctggcaacttctcccccaggtgccttccccctgctgccggcctttgctccttcacttccaacatctctcaaaataaaaatccctcttcccgctctgagcgattcagctctgcccgcagcttgtacatgtctctcccctggcaaaacaagagctgggtagtttagccaaacggcaccccctcgagttcactgcagacccttcgttcaccgtgtcacacatagaggggttctgagtaagaacaaaacgttctgctgctcaagccagtctggcaagcactcagcccagcctcgaggtccttctggggagagtgtaagtggacagagtcctggtcagggggcaggagtgtcccaagggctggcccacctgctgtctgtctgctcctcctagcccttggtcagatggcagccagagtccctcaggacctgcagcctcgccccggcagaagtcttttgtccaggaggcaaaaagccagagattctgcaacacgaattcgaagcaaacaaacacaacacaacagaattcctggaaagaagacgactgctaagacacggcaggggggcctggagggagcctccgactctgagctgctccgggatctgccgcgttctcctctgcacattgctgtttctgcccctgatgctggagctcaaggagactccttcctctttctcagcagagctgtagctgactgtggcattactacgcctccccacacgcccagacccctcactccaaaatcctactggctgtagcagagaatacctttgaaccaagattctgttttaatcatcatttacattgttttcttccaaaggccccctcgtataccctccctaacccacaaacctgttaacattgtcttaaggtgaaatggctggaaaatcagtatttaactaataaatttatctgtattcctctttc

SEQ ID NO: 8 - protein, 443 aa, Homo sapiens
MYQSLAMAANHGPPPGAYEAGGPGAFMHGAGAASSPVYVPTPRVPSSVLGLSYLQGGGAGSASGGASGGSSGGAASGAGPGTQQGSPGWSQAGADGAAYTPPPVSPRFSFPGTTGSLAAAAAAAAAREAAAYSSGGGAAGAGLAGREQYGRAGFAGSYSSPYPAYMADVGASWAAAAAASAGPFDSPVLHSLPGRANPAARHPNLVDMFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAAPSGSESLPPASGASSNSSNATTSSSEEMRPIKTEPGLSSHYGHSSSVSQTFSVSAMSGHGPSIHPVLSALKLSPQGYASPVSQSPQTSSKQDSWNSLVLADSHGDIITA

SEQ ID NO: 9 - RNA, 2780 nt, Homo sapiens
  source 1..2780: mol_type = mRNA
agctgtacatggagatcttgctgggaaaatccgcttgctcccctcacgtcgtccagcccaggagaaccaccgccgtcaccccggagcttcctcggccaccgcgcagagccctccgagagcccgagccgcggtcttcgagctccaaggctcattcagggccccagatccttgccccgaaaggagaggatctgagaaaatggatgcactgagacctctctgaaaaccctccgagagagcgcgagaggagcgaggacacgttactcgcagctaaaatcacatttaaggaccaaaacaacaacaaccaaaaatttcattaaaacaataagcgcccaagaacccagatcgggctggtggggggaggggaagaggcgggaaggggagggtcgcacggaggtagctttgcagtgagcagtcgaccccgccgccccccggcacagctggaccggctcctccagccgcggctcagactcgcccctggattccgggttagcttcggtgccaggaccgcggcccgggcttggattcccgagactccgcgtaccagcctcgcgggagccccggcacctttgtatgagcacgagaggattctgcctccgcgcagcagcccgggaagcaggagccgaagcgcgggccgtggagcaaggcgggaaccggaggcggcggcggcggcggccaggggcgcacggtgccaggaccagctcgccgcgccccatggggagccggcggccgcagcgctgctgaggcgggcccggctggccaggcggggggacggggcccgggctgcagcagccccctctgcggctgccgggcgggcccgggcgcccgggggctggggggtggggggtgggggaggacgccgagcgctgaggcaggggcccgggccgagggcgcggcggggctgcgcgcacgctggggcgcgtggaggggcgcggagggcgaaatgagtctggtaggtggttttccccaccacccggtggtgcaccacgagggctacccgtttgccgccgccgccgccgcagctgccgccgccgccgccagccgctgcagccatgaggagaacccctacttccatggctggctcatcggccaccccgagatgtcgccccccgactacagcatggccctgtcctacagccccgagtatgccagcggcgccgccggcctggaccactcccattacgggggggtgccgccgggcgccgggcccccgggcctgggggggccgcgcccggtgaagcgccgaggcaccgccaaccgcaaggagcggcgcaggactcagagcatcaacagcgccttcgccgaactgcgcgagtgcatccccaacgtacccgccgacaccaaactctccaaaatcaagaccctgcgcctggccaccagctacatcgcctacctcatggacctgctggccaaggacgaccagaatggcgaggcggaggccttcaaggcagagatcaagaagaccgacgtgaaagaggagaagaggaagaaggagctgaacgaaatcttgaaaagcacagtgagcagcaacgacaagaaaaccaaaggccggacgggctggccgcagcacgtctgggccctggagctcaagcagtgaggaggaggagaaggaggaggaggagagcgcgagtgagcaggggccaaggcgccagatgcagacccaggactccggaaaagccgtccgcgctccgctctgaggactccttgcatttggaatcatccggtttatttatgtgcaatttccttcccctctctttgaccccctttgaggcatctgctccccgtctccccctccaaaaaaaaagtggatatttgaagaaaagcattccatattttaatacgaagaggacactcccgtgtggtaagggatcccgtcgtctcatagattctgtgtgcgtgaatgttccctcttggctgtgtagacaccagcgttgccccccgccaacctactcaaccccttccagataaagacagtgggcactagtgcgtttgtgaagtgtatctttaatacttggcctttggatataaatattcctgggtattataaagttttatttcaaagcagaaaacagggccgctaacatttccgttggggtcggtatctagtgctatccattcatctgtggtcgttccctctttgaagatgtttccaacagccacttgttttgtgcacttccgtcctctaaaactaaatggaatttaattaatattgaaggtgtaaacgttgtaagtattcaataaaccactgtgttttttttttacaaaaaccttaatcttttaatggctgatacctcaaaagagttttgaaaacaaagctgttatacttgttttcgtaatatttaaaatattcagaagtaaactaaattatcatgattgcctctaactttattttaaagactcagtggttccaaccagtcaccctgacctgcggcctacgcaggaggaggaggtgctcttaaagagaagtgtccttgttacaaatcctgcaaatggtctggggtttgtcggtgtggtgtctccttccctcttcccccagctggagaacgctgagtagtctctagaaggaagatctgggctggagaacccagtccggcagttcgctcagaaggtgtaaaggtgctcttgctttcctgaagtcaatcagaagccatttcttgaggccgtcagtttttgtttggagagtgtttctggtggaggagttgtgaggagaaccccggcattattgctgcaacgggaactagtctggggtgtttaattcaaactatggggctttcatccaagaa

SEQ ID NO: 10 - protein, 217 aa, Homo sapiens
MSLVGGFPHHPVVHHEGYPFAAAAAAAAAAAASRCSHEENPYFHGWLIGHPEMSPPDYSMALSYSPEYASGAAGLDHSHYGGVPPGAGPPGLGGPRPVKRRGTANRKERRRTQSINSAFAELRECIPNVPADTKLSKIKTLRLATSYIAYLMDLLAKDDQNGEAEAFKAEIKKTDVKEEKRKKELNEILKSTVSSNDKKTKGRTGWPQHVWALELKQ

SEQ ID NO: 11 - RNA, 8565 nt, Homo sapiens
  source 1..8565: mol_type = mRNA
aatcgccggcagcctatgacatcagacaggaacgcctgggatgccgcgctgctcctggccaacctccgaggaggaggagggtcccgccggctaagagttaattagccccgcacggcgaggggggaggcgccagttttctggggacactggctgccactgtactcctacccaggggagctcacggagagttggatgaattctgggttgttagctgcggtcagctgggctcccgggagcctgttgctggtggagaacagggggcgcctggccaagggaccagcggcttgctgagactcaacatgacactcctggggtctgagcattccttgctgattaggagcaagttcagatcagttttacagttaagacttcaacaaagaaggacccaggaacaactggctaaccaaggcataataccaccactgaaacgtccagctgaattccatgagcaaagaaaacatttggatagtgacaaggctaaaaattccctgaagcgcaaagccagaaacaggtgcaacagtgccgacttggttaatatgcacatactccaagcttccactgcagagaggtccattccaactgctcagatgaagctgaaaagagcccgactcgccgatgatctcaatgaaaaaattgctctacgaccagggccactggagctggtggaaaaaaacattcttcctgtggattctgctgtgaaagaggccataaaaggtaaccaggtgagtttctccaaatccacggatgcttttgcctttgaagaggacagcagcagcgatgggctttctccggatcagactcgaagtgaagacccccaaaactcagcgggatccccgccagacgctaaagcctcagataccccttcgacaggttctctggggacaaaccaggatcttgcttctggctcagaaaatgacagaaatgactcagcctcacagcccagccaccagtcagatgcggggaagcaggggcttggcccccccagcacccccatagccgtgcatgctgctgtaaagtccaaatccttgggtgacagtaagaaccgccacaaaaagcccaaggaccccaagccaaaggtgaagaagcttaaatatcaccagtacattcccccagaccagaaggcagagaagtcccctccacctatggactcagcctacgctcggctgctccagcaacagcagctgttcctgcagctccaaatcctcagccagcagcagcagcagcagcaacaccgattcagctacctagggatgcaccaagctcagcttaaggaaccaaatgaacagatggtcagaaatccaaactcttcttcaacgccactgagcaatacccccttgtctcctgtcaaaaacagtttttctggacaaactggtgtctcttctttcaaaccaggcccactcccacctaacctggatgatctgaaggtctctgaattaagacaacagcttcgaattcggggcttgcctgtgtcaggcaccaaaacggctctcatggaccggcttcgacccttccaggactgctctggcaacccagtgccgaactttggggatataacgactgtcacttttcctgtcacacccaacacgctgcccaattaccagtcttcctcttctaccagtgccctgtccaacggcttctaccactttggcagcaccagctccagccccccgatctccccagcctcctctgacctgtcagtcgctgggtccctgccggacaccttcaatgatgcctccccctccttcggcctgcacccgtccccagtccacgtgtgcacggaggaaagtctcatgagcagcctgaatgggggctctgttccttctgagctggatgggctggactccgagaaggacaagatgctggtggagaagcagaaggtgatcaatgaactcacctggaaactccagcaagagcagaggcaggtggaggagctgaggatgcagcttcagaagcagaaaaggaataactgttcagagaagaagccgctgcctttcctggctgcctccatcaagcaggaagaggctgtctccagctgtccttttgcatcccaagtacctgtgaaaagacaaagcagcagctcagagtgtcacccaccggcttgtgaagctgctcaactccagcctcttggaaatgctcattgtgtggagtcctcagatcaaaccaatgtactttcttccacatttctcagcccccagtgttcccctcagcattcaccgctgggggctgtgaaaagcccacagcacatcagtttgcccccatcacccaacaaccctcactttctgccctcatcctccggggcccagggagaagggcacagggtctcctcgcccatcagcagccaggtgtgcactgcacagaactcaggagcacacgatggccatcctccaagcttctctccccattcttccagcctccacccgcccttctctggagcccaagcagacagcagtcatggtgccgggggaaacccttgtcccaaaagcccatgtgtacagcaaaagatggctggtttacactcttctgataaggtggggccaaagttttcaattccatccccaactttttctaagtcaagttcagcaatttcagaggtaacacagcctccatcctatgaagatgccgtaaagcagcaaatgacccggagtcagcagatggatgaactcctggacgtgcttattgaaagcggagaaatgccagcagacgctagagaggatcactcatgtcttcaaaaagtcccaaagatacccagatcttcccgaagtccaactgctgtcctcaccaagccctcggcttcctttgaacaagcctcttcaggcagccagatcccctttgatccctatgccaccgacagtgatgagcatcttgaagtcttattaaattcccagagccccctaggaaagatgagtgatgtcacccttctaaaaattgggagcgaagagcctcactttgatgggataatggatggattctctgggaaggctgcagaagacctcttcaatgcacatgagatcttgccaggccccctctctccaatgcagacacagttttcaccctcttctgtggacagcaatgggctgcagttaagcttcactgaatctccctgggaaaccatggagtggctggacctcactccgccaaattccacaccaggctttagcgccctcaccaccagcagccccagcatcttcaacatcgatttcctggatgtcactgatctcaatttgaattcttccatggaccttcacttgcagcagtggtagaatgcccaatgcaccagtgctatggaagaccaatggagttccatgggggaaagcacacagccatacatactttactgtccaaaaacagaagaagaagaagagaattaaaaagaagcaatgatttctgtgccaatgaacaagaacaaaagtcatttttagaaatacatatactgtaatatttaccaacagtcagtaactgttaatgatttcaacaatgcattaaaagaatgtgctttctcagattaaggatgccaaaaaagatatttcactgccttttcaaagaccagtatattttctagcccataatttttctcaggcattgttggggcataagctcacactgtaagcttttctcatgaattcactagacataacgtggaaggaaaacgtagtcttttgggagtacagggaagccagcccctcaaagcttatggaagacatacctgcaatggaagctgttgcccaatgtctccattactatctttcaaaagagaagccagacccagcttcagatcaaaagttcttgagacagaggaacaaaaccaatcgatttccagggaagctaatcaactctcttttccctctaccacaaaactgccctgctggagtggttctgaacctgtacccaggactcgatgtggtcactaataacaattaacctgaactgagtccacagaactccactcggaactttcttcttttttaactagtggcccaatcattcccaccatctctgtgctgataagtacgtgtcctagatgagaaccctgaagaatgcagaccttcttcccccgaaggagatgccacaagctctccaacacagccccctttagttccaaagactagagatgaccacattggtagaagtatatctcgaggcacaggaagggagccccaccagggataattcagacaggactagagaataacatcatttcacataccctgggataaacaccctgggttcctatagaaggactattacttatgggagtccaacttctccttttgttttgttattatcagtttatctttctcccactccacttttccttcaaggtaccaatcctttcctgttcctcgtttggccatctttctttttctgcctccacattgggaggggaggacttctcagttctaacaagctgccatactcctaagaaagccatttttgaaaaatttaacaatccaggttcttctggagaactcattctccacacgcacagtttgctgcaaaaggaagttgcaagaatttcttgaggaagaaactggtgacttggtccatcagtcacgaagttctttctattctcgtttagttttcaagaaattattggtttgtgttgctctggggaaattggaaatcattacattgtaaagacaaatatggatgatatttacaagagagaatttcagatctgggtttttgaaagaaaacagaattgcgcattgaaaacgatggaaggaaaaagacaatggtctaatgtgcattcctcattacctctcgtggctttggctgggagttggaaaaagctaaaatttcagaacagtctctgtaaggctctctgtggctccagttcaccattttatattgttgcatgctgtagaaaggagctattgctgttgttttgtttttttatttaaatcactaaggcactgtttttatcttttgtaaaaaaaaaaaaaaagttgttcactgtgcacttatagaaaaaataatcaaaaatgttgggattttagaagctctctttttgataaaccaaagatttagaagtcattccattgttaacttgtaaaaatgtgtgaacacagagagtttttggtgattgctactctgaaagctgccagatcttattctgggggtgggatgtggaggaatacacatacacacacaaacatacatgtatgtataatagatatatacatatgtgtatattatatctgtgtgtgcatgtatctccaaaagcggcgttacagagttctacaccaaaagcctttaacccttaatctgctgtgaatgatacctggcctttctcactatgaatttctgattaaccaaccagactacacgttgcctctctgtgtatgactaacggctccaacccgatgactcacagctacttgcttatcgtgaacaagctcatcttggcaatgaatatggatgtgaaaagacagaacagcttcaccattagtagctggaaatggtatcacagtctcttatagaggaatatgaaaggaacaagaaaatcattttacattccttttatctgtattgtgctttaaaagatccacatggtaaattttttattttgcttttatgtcagtcatcagaaccaaaaaaatccagaagaaaaaattgccagtgtttcctttgaagatgaagctactggggaagaaaaccttattaatacactccacacatttgttcattcctcagctgttggtgttttcttggggtcttgacaaagcttgctggtcagtgcacttttcaggtgtcacgttttgctgtttgtatgttttttcttccccttacttcctttggaaaacaaactcacacagtgcccctactctgagacctgggactgagtgttaattattttttccttgggtatttctatctgagagactagacctagttaggaggcctctgtacttctccagattgtacctttttatggggatctttgaggctatgacccaggactgatagatatgccttacggaagacaaaagataaaatggttcctatatcctaatgcaaaccaacacagttaaaagagcagatctctggataactgctctcaacctgcttctacagtctccacaaaccgcattcaccctctctcttcatagctcagacatgaaatttgagggagaaaactggagataattgggagaaaattgatgaagttggctgcttccagtagatcagataatccatgaatttgtctcccattgagaattttattttaaattcttttaaactcttcgttgtgtcttttgtgatgacaaatcaggcatgactaaaagatgtacagagacttacgaagatggtcacattcaagttccctaatgctcttagaacctgaagatgaccatgtgtagttttcttaagacctctgaacccccatggtgatgaagacttgaagacatttgcagctatctgctgcagtctggtagattcatacttatctaaagaagtcaaaaaatttattcgtgcaagtgcttgcaggaagccagtgcttattagtagtgaccctgcttctatcaacgttattgagacaacacatattctattctaagggagaaagagggaggaagagagggagggagggaggaagaagagggagggagcgaggaaggaagataggagatgggtaggggggtaaagagaaaaggagggagaagggaaggaaggaaagaagagaggaaagaaaggagggaaggaaaaaagggccaaactttctgatctatgaacttctcagttcagctgtcacattatgagaagtaaatcagaatttttttaaggagaagtcattcttagcactacaataattgtaccagtaattgaggaaaccaagacaatcttcacctgaataatagagggtctgagaactgtcagccttttgccattcaaaaacatttatgtccaacctgaaaaaaaagcatcaataaaacctatcccaagcattcaaaatagtcctttccaaatgttatttattttaaagtcaatcagctcttttagaaacagattctggtctggctgaaaactcccacaacaaatttactcatccagtggctaatatttaatgcccaccatgggcagagcacacaaatcttcagataaacaatactgaattgattcagcacagattgttcagatttgaatgaccagggagttgtatttgcacatgcaagaacactaagaatctcccagtcctcaaactagaaacctttcagctacgatgaaaaaaaaaaaggggtcttcatttttccaagagggggtggaggtggggatcactttttagctaaaagctatctctcacttcaaaattcttgtctttttctttgtggacaaacaccagtagtctatcacttggagatcttttaatatctcccatcatttaaaacatccacgagagtttgaagatttgtgttgattgccagatacagaagccccttgaaaataaggaaagggtggaggaagcatttttgtgtcctatccctacttatcgtagcagctctatagacaaaagggacacttactggtgagcctctggcccttaaaagaaaatcatctaagaatatgaaggcaatttgatttccccccacagccctcagctgccttcctcacagaaggaagttcccaaaattgctggtacacagtttgcaatcaaatatcagatatgagaaaacctgtagtgaagagtctgggttcttggttttctcataaatccaatataaatttgtaggttggttcagggtcaaaattgccagtgctttattagacagatgatactgatagacacacagagcccaggtcctggaacaagacaatcctgtagtgccaagatctggtcagttgcgttaaggagctgggtttgattctagagtccaggtttatagagaaaccctggctagattgagcctacccatggggagacgatttcaagacaggatgagatctgggaagaattttgttgtcatctgccagggaaattatcacaggactcattgaatgcaataacatgtgagtaagttcccttttgattctgggaatcagcgattttccctgtggattaagacaaaccaacgccagaaggtctcctgtgcttattttaaccatctgctcccatcgtgaaccctggagcatgcatttcctagaagtggtttcatagctcctgtgtgttcatggaaaaggggagtataatgatggggatgctggaagcttttttaatgttttccaaaggaaaggaacccacactgctccccagagttcctttccaatggccctgcagtaagaacggaggacaatgtattgctgggtgcttaaaatcctccctcagtgaagcacaaagagacactttgtaaagaaaaaaagagcaagcataggttctctgtgggaccttgtggagtggtgttttcacgttggtctctttggctcaattgagcataatcagaaagaaatgtgggttattgggaagagacaaaaagcagtggctaaaataccaaagttggcatgtgttcttttttaaaaaaaaaaaaaaatgcatatatttttaaataaaatgtttattttaaaaagaaa

SEQ ID NO: 12 - protein, 986 aa, Homo sapiens
MTLLGSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSSTSALSNGFYHFGSTSSSPPISPASSDLSVAGSLPDTFNDASPSFGLHPSPVHVCTEESLMSSLNGGSVPSELDGLDSEKDKMLVEKQKVINELTWKLQQEQRQVEELRMQLQKQKRNNCSEKKPLPFLAASIKQEEAVSSCPFASQVPVKRQSSSSECHPPACEAAQLQPLGNAHCVESSDQTNVLSSTFLSPQCSPQHSPLGAVKSPQHISLPPSPNNPHFLPSSSGAQGEGHRVSSPISSQVCTAQNSGAHDGHPPSFSPHSSSLHPPFSGAQADSSHGAGGNPCPKSPCVQQKMAGLHSSDKVGPKFSIPSPTFSKSSSAISEVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQW

SEQ ID NO: 13 - RNA, 2957 nt, Homo sapiens
  source 1..2957: mol_type = mRNA
ggcggcggcaggaccgagcgcggcaggcggctggcccagcgcacgcagcgcggcccgaagacgggagcaggcggccgagcaccgagcgctgggcaccgggcaccgagcggcggcggcacgcgaggcccggccccgagcagcgcccccgcccgccgcggcctccagcccggccccgcccagcgccggcccgcggggatgcggagcggcgggcgccggaggccgcggcccggctaggcccgcgctcgcgcccggacgcggcggcccgtctggggagcagggaggagccctgtgccctgtcccaggatccatgggtaggaacaccatggacagggagagcaaacggggccatctgtcaccaggggcttagggaaggccgagccagcctgggtcaaagaagtcaaaggggctgcctggaggaggcagcctgtcagctggtgcatcagaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaa

SEQ ID NO: 14 - protein, 480 aa, Homo sapiens [sequence omitted]